(19) United States
(12) Patent Application Publication
(10) Pub. No.: US 2021/0363507 A1
(43) Pub. Date: Nov. 25, 2021

(54) Gene Targeting

(71) Applicant: Algentech SAS, Evry Cedex (FR)

(72) Inventors: Alexander Sorokin, Evry Cedex (FR); Isabelle Malcuit, Evry Cedex (FR); Florina Vlad, Evry Cedex (FR)

(73) Assignee: Algentech SAS, Evry Cedex (FR)

(21) Appl. No.: 15/733,853

(22) PCT Filed: May 31, 2019
(86) PCT No.: PCT/EP2019/064236

§ 371 Date: Nov. 30, 2020

(30) Foreign Application Priority Data
Jun. 1, 2018 (EP) 18175634.7

(51) Int. Cl. C12N 9/22; C12N 15/90; C12N 15/11

(52) U.S. Cl. CPC C12N 9/22; C12N 15/907; C12N 2800/80; C07K 2319/85; C12N 2310/20; C12N 15/11

(57) ABSTRACT

Methods, reagents and compositions for providing more accurate and reliable genetic modification are provided. In particular, a nucleic acid encoding a fusion protein comprising a 5′ to 3′ DNA exonuclease domain and an RNA binding domain is described. Also provided are methods, reagents and compositions for in vivo genetic modification of the genome of a human or animal cell. Furthermore, the present application relates to uses of the said methods, reagents and compositions in the treatment of disease and production of transgenic animals.

This application is a U.S. national stage application filed pursuant to 35 U.S.C. § 371 from International Patent Application PCT/EP2019/064236, filed on May 31, 2019 which claims the benefit of priority and the filing date of European Patent Application EP 18175634.7, filed on Jun. 1, 2018, the content of each of which is hereby incorporated by reference in its entirety.

The present invention relates to methods, reagents and compositions for providing more accurate and reliable genetic modification. The invention further provides methods, reagents and compositions for in vivo genetic modification of the genome of a human or animal cell or complementation of the inherited mutations in such a host using a correct copy of the native gene. Furthermore, the present invention relates to uses of the said methods, reagents and compositions in the treatment of disease and production of transgenic animals.

In recent times genetic modification by way of random mutagenesis has given way to directed mutagenesis of particular nucleotide sequences using sequence-specific protein complexes.

Examples of such protein complexes include zinc-finger nucleases (ZFN), transcription activator-like effector nucleases (TALENs), complexes derived from the CRISPR-Cas9 system of Streptococcus pyrogenes and other bacteria, and CRISPR-Cpf1.

ZFNs and TALENs are both protein nucleases whose protein structure allows them to interact with and recognise a particular DNA sequence before cutting the DNA at a defined location. Thus cutting a particular DNA sequence requires a uniquely designed ZFN or TALEN protein.

In contrast, the CRISPR-Cas9 and CRISPR-Cpf1 systems use a single protein whose activity is directed by an RNA cofactor whose nucleotide sequence defines the location at which the endonuclease will act to produce a double strand break.

Thus, all of these protein complexes act by making a DNA double strand break at a predefined DNA sequence. This double strand break is then normally repaired by the non-homologous end joining (NHEJ) pathway.

Repair by NHEJ is highly efficient and rapid but is more error-prone than the alternative pathway for repair of DNA double-stranded breaks which is homology-directed repair (HDR). Consequently, a proportion of NHEJ pair of events will cause insertion or deletion of nucleotides at the break site. Such insertion or deletion events are known as “indels”.

Homologous recombination proceeds in several distinct stages: the earliest step is processing of the DNA end to produce 3′ single-stranded DNA (ssDNA). Following 5′ strand resection, the 3′ ssDNA is bound by RecA-type recombinases that catalyzes homologous pairing and DNA strand exchange. The 3′ end then primes DNA synthesis, and resolution of Holliday junctions or strand annealing between newly-synthesized ends results in repair of the initial DSB (Seitz et al., 2001, PMID: 11677683).

Alternatively, larger genetic modifications are enabled by the presence of a donor DNA molecule in the vicinity of an artificially-created DNA double-stranded break. In this instance HDR of the induced DSB causes repair of the DSB using the sequence of the donor molecule. In this way specific modifications can be made and short sequence insertions are also possible. One example of such a donor and a vector for producing large amounts of such donor molecules is disclosed in WO 2010/084331.

However, the efficiency of genetic modification using HDR is low because most repair of double strand breaks proceeds via the more rapid NHEJ pathway.

Furthermore, while the above-mention protein complexes are directed to specific sequences their endonuclease activity has been known to act at other sites. Such “off-site breaks” are particularly a problem as NHEJ is more error prone.

Thus, there exists a need for alternative and preferably improved methods and reagents for sequence-specific modification of nucleic acid sequences and of DNA sequences in particular. Furthermore, there is a need for techniques and reagents that more reliably and efficiently yield the desired genetic modification or complement for specific mutation(s) in the host genome. Additionally, there is a need for techniques and reagents that reliably allow insertion of longer DNA sequences at a pre-defined locus.

An object of the present invention is to provide reagents and techniques for using these reagents that provide alternatives, and in particular embodiments allow more reliable, efficient and accurate modification or complementation for mutation in a target genome at specific loci within the genome.

There are provided herein proteins, protein-nucleic acid complexes and vectors that provide improved transformation efficiencies and methods for carrying out such transformations. Furthermore the said methods, reagents and compositions may be used for in vivo genetic modification of the genome of a human or animal cell. Furthermore, the present invention relates to uses of the said methods, reagents and compositions in the treatment of disease.

Accordingly, the present invention provides a nucleic acid encoding a first fusion protein comprising a 5′ to 3′ DNA exonuclease domain and an RNA binding domain. As an alternative to the first fusion protein, in alternative embodiments the invention utilises a 5′ to 3′ DNA exonuclease without an RNA binding domain.

Functionally significant domains or regions of different proteins or polypeptides may be combined for expression from an encoding nucleic acid as a fusion protein. For example, particularly advantageous or desirable properties of different proteins or polypeptides may be combined in a hybrid protein, such that the resultant expression product, may include fragments of various parent proteins or polypeptides.

In the fusion proteins described herein the domains of the fusion proteins are preferably joined together via linker peptides. The particular choice of linker will depend on the constituent domains of the fusion protein. The suitability and choice of appropriate linker peptides is discussed in Chen et al. (Adv Drug Deliv Rev. 2013; 65(10): 1357-1369).

The first fusion protein may be for transformation of a eukaryotic cell in concert with an RNA-guided endonuclease.

Tethering of proteins to RNAs by bacteriophage proteins has been established (Baron-Benhamou et al., 2004, doi:10.1385/1-59259-750-5:135, Coller & Wickens, 2007, doi: 10.1016/S0076-6879(07)29014-7; Keryer-Bibens et al., 2008, doi:10.1042/BC20070067, Tsai et al., 2011, doi: 10.1074/mcp.M110.007385). A number of stem-loops and bacteriophage coat proteins are available for tethering, such as MS2 stem loop (SEQ ID NO: 1)-MS2 coat protein (SEQ ID NO: 2) (Peabody, 1993, PMID: 8440248), PP7 stem loop (SEQ ID NO: 3)-PP7 coat protein (SEQ ID NO: 4) (Lim & Peabody, 2002, PMID: 12364592), B-box stem loop (SEQ ID NO: 5)-lambda N coat protein (SEQ ID NO: 6) (Keryer-Biben et al., 2008, doi: 10.1042/BC20070067).

Tethering customized sgRNA from CRISPR with the bacteriophage coat protein-binding RNA stem-loop is known, wherein a stem-loop RNA structure was introduced inside or at the 3′ end of sgRNA and a potential protein of interest was fused to bacteriophage coat protein (Konermann et al., 2015, doi: 10.1038/nature14136; Nowak et al., 2016, doi: 10.1093/nar/gkw908; Park et al., 2017, doi: 10.1371/journal.pone.0179410, Anton et al., 2018, doi: 10.1093/biomethods/bpy002) for site-specific visualization of genomic elements, transcriptional regulation and epigenetic manipulation.

Both Zalatan et al. (Cell (2015) 160, 339-350) and the CRISPRainbow system described initially by Ma et al. (Nat Biotechnol. 2016 Apr. 18. doi: 10.1038/nbt.3526) utilise a modified sgRNA containing 3′ RNA hairpin aptamers that bind uniquely labelled RNA binding proteins. Thus the sgRNA is functionalised so that it can be used to locate fusion proteins comprising binding domains for the aptamers in association with the sgRNA (SEQ ID NO:s 7 and 8) and hence the endonuclease it is associated with.

The action of the first fusion protein may be for inhibition of NHEJ during transformation of a cellular genome so as to promote HDR. The effect of such 5′ to 3′ resection on DNA double-strand breaks is to suppress religation of DNA breaks (i.e. by blocking NHEJ), by producing a substrate that is less suitable for NHEJ but is significantly more suitable for loading of host recombinases and modification of the locus using HDR. Thus the action of the first fusion protein may be for inhibition of NHEJ during transformation of a cellular genome so as to promote HDR.

Lambda exonuclease (λ-exo) plays an important role in the resection of DNA ends for DNA repair. Lambda exonuclease is a 5′→3′ exonuclease that progressively digests one strand of a duplex DNA molecule to generate a 3′-single stranded-overhang (Carter& Radding, 1971, PMID: 4928646). Because of its robust properties and low cost, λ-exo is widely used in multiple biotechnology applications, such as genetic engineering using homologous recombination.

In the complex with DNA, λ-exo unwinds two bases at the 5′ end of the substrate strand to pull it into the reaction center. It hydrolyses double-stranded DNA (dsDNA) 130 times faster than single-stranded DNA (ssDNA) (Little, 1967, PMID: 6017737). A DNA duplex with a 5′ phosphorylated blunt or recessed end is the appropriate substrate for λ-exo, while the digestion rate of a dsDNA with a 5′ hydroxyl end or a 5′ phosphorylated overhang is significantly slower (Mitsis&Kwagh, 1999, PMID:10454600, et al., 2018, doi: 10.1093/nar/gky154).

Exonucleases with 5′-3′ activities are presented in other organisms and 5′-3′ exonucleases can be used in general for the invention. The Cas4 protein is one of the core CRISPR-associated (Cas) proteins implicated in the prokaryotic CRISPR system for antiviral defense. The Cas4 protein is a 5′ to 3′ single stranded DNA exonuclease in vitro and it is involved in DNA duplex strand resection to generate recombinogenic 3′ single stranded DNA overhangs (Zhang et al., (2012) https://doi.org/10.137/journal.prone.0047232).

RecJ from Deinococcus radiodurans, a member of DHH family proteins, is the only 5′ nuclease involved in the RecF recombination pathway, providing the resection of DNA strand with a 5′ end at double-strand breaks as an essential step in recombinational DNA repair. As a processive nuclease, RecJ only degrades ssDNA in a 5′-3′ direction but nuclease alone is capable of digesting DNA with only 5′-ssDNA overhang (Jiao et al., 2012, doi:10.1016/j.dnarep.2011:11.008).

Genetic studies in Saccharomyces cerevisiae show that end resection takes place in two steps. Initially, a short oligonucleotide tract is removed from the 5′ strand to create an early intermediate with a short 3′ overhang by the highly conserved Mre11-Rad50-Xrs2 (MRX) complex and Sae2. Then in a second step the early intermediate is rapidly processed generating an extensive tract of ssDNA by the exonuclease Exo1 and/or the helicase-topoisomerase complex Sgs1-Top3-Rmi1 with the endonuclease Dna2 (Mimitou& Symington, 2011, doi: 10.1016/j.dnarep.2010.12.004).

In archaea, such as Pyrococcus furiosus the end resection is executed by the bipolar helicase HerA and the 5′-3′ exonuclease NurA (Hopkins&Paull, 2008, doi: 10.1016/j.cell.2008.09.054). Thus, loading or activation of HerA-NurA complex promotes resection of the 5′ strand of the double-stranded DNA break (DSB) and initiate of strand invasion.

For more information on enzymes involved in 5′ end DNA resection and mechanisms of 3′ DNA ends generation in the three domains of life see Blackwood et al., 2013, (doi: 10.1042/BST20120307), Liu & Huang, 2016, (doi: 10.1016/j.gpb.2016.05.002); Raynard et., 2019, (doi/10.1101/gad.1742408); Sharad & You, 2016, (doi:10.1093/abbs/gmw043); Yin & Petes, 2014, (doi.org/10.1534/genetics.114.164517).

The exonuclease may be a dsDNA exonuclease. The exonuclease is suitably a 5′ to 3′ exonuclease and is involved in recombination, double-strand break repair, the MMS2 error-free branch of the post replication repair (PRR) pathway and DNA mismatch repair. Preferably the exonuclease is the λ-exo protein from bacteriophage lambda (SEQ ID NO: 9). Without wishing to be bound by theory, this enzyme can produce approximately 100-150 bp 3′ overhangs at dsDNA break sites during methods of the invention. The exonuclease may be also used without a RNA-binding domain, though efficiency of HDR may be slightly reduced.

The invention also provides a nucleic acid encoding a second fusion protein comprising an endonuclease domain and, e.g. is fused to, a binding domain for an origin of replication.

The endonuclease may cleave a target nucleic acid molecule in a sequence specific manner. The sequence specific cleavage of the nucleic acid molecule may be double or single stranded (including ‘nicking’ of duplexed nucleic acid molecules). Double stranded cleavage may yield blunt ends or overhanging termini (5′ or 3′ overhangs). The sequence specific nuclease preferably acts as a monomer but may act as a dimer or multimer. For instance a homodimer wherein both monomers make single strand nicks at a target site can yield a double-strand break in the target molecule. Preferably the cleavage event makes a double-stranded break in the target molecule.

Examples of sequence-specific endonucleases include zinc-finger nucleases (ZFN), transcription activator-like effector nucleases (TALENs), complexes derived from the CRISPR-Cas9 system of Streptococcus pyrogenes and other bacteria, and CRISPR-Cpf1.

A nucleic acid molecule may comprise double- or single-stranded DNA or RNA. The nucleic acid molecule may also comprise a DNA-RNA duplex. Preferably the nucleic acid molecule is double-stranded DNA. Preferably the cleavage event makes a double-stranded DNA break in the target molecule.

Preferably the endonuclease is a DNA endonuclease and most preferably this is Cas9 or Cpf1 from Acidominococcus or Lachnospiraceae. This may be Cas9 from Streptococcus pyrogenes (SEQ ID NO: 10) or a homologous or functionally equivalent enzyme from another bacteria.

The fusion protein may comprise an endonuclease and a component of the replication initiation complex or replication complex, e.g. encoded by a viral Rep gene.

The components of the replication initiation complex or replication complex are necessarily associated with origins of replication and may be covalently attached thereto or to the elongating nucleic acid molecule. Suitably the origin of replication is derived from a virus. Most suitably the virus is circovirus or another member of the circoviridae, such as the genera circovirus, anellovirus and cyclovirus. Preferably the virus is porcine circovirus 1 (PCV1) or a non-enveloped human DNA torque teno virus (TTV). However, other members of the circoviridae are found in a large number of bird and mammal hosts can may also be used.

The use of porcine circovirus or torque teno virus (TTV) is advantageous because these viruses are non-pathogenic in humans.

The PCV1 Rep protein (SEQ ID NO: 11) binds to the PCV1 origin of replication (SEQ ID NO: 12) and thus becomes covalently linked to the ssDNA strand of donor DNA produced by rolling circle replication initiated at the origin of replication. Thus the newly replicated donor DNA molecule is covalently linked to the second fusion protein and is necessarily brought into close proximity to the site of the double-stranded DNA break caused by the endonuclease.

Targeting of donor DNA to the target is a critical factor for HDR. A number of methods have been developed for donor DNA tethering to the target (Sharma & McLaughlin, 2002, doi: 10.1021/ja020500n; Aird et al., 2018, doi: 10.1038/s42003-018-0054-2; Savic et al., 2018, doi: 10.7554/eLife.33761). It is interesting that covalent link of donor DNA to cas9 fusion protein increases efficiency of homology-dependent recombination by 24-30 folds, as indicated by fusion of HUH endonucleases to cas9 (Aird et al., 2018, doi: 10.1038/s42003-018-0054-2) or cas9-SNAP-tag domain fusion (Savic et al., 2018, doi: 10.7554/eLife.33761).

The tethering of donor DNA to the target is, however, technically challenging, as (i) single-stranded linear DNA (ssIDNA) should be produced in vitro, (ii) ssDNA delivery to cell is less efficient then dsDNA, (iii) ssIDNA is not stable in vivo and is subjected to rapid endonuclease degradation, and as result, (iv) low concentration of donor DNA around the targeted locus significantly reduce HDR.

Thus delivery of ssDNA to cells is challenging. ssDNA is difficult to deliver technically because ssDNA is not naturally imported into cells and is rapidly degraded. Advantageously, the present invention addresses this problem by delivering dsDNA and then producing ssDNA in the desired location from this dsDNA

To address these issues the invention can utilise HUH rep proteins from bacteriophages, circoviruses, geminiviruses, rolling circle transposons from bacteria or plants (such as helitrons) preferentially active in mammalian cells for rolling circle replication, and replicative donor vector containing double-stranded donor DNA flanked by one or two viral origins of replication.

Modification of the target is significantly improved by producing ssDNA in vivo and causing it to accumulate in the vicinity of the locus to be modified. Accumulating the ssDNA in the vicinity of the locus to be modified means that it is available for use in HDR processes for a longer period, which advantageously promotes HDR. Additionally, amplification of the ssDNA copy number allows more of the ssDNA moiety to accumulate close to the locus of interest, which, as noted above, promotes more efficient editing of the target locus.

Our approach allows addressing all problems indicated above by one or more or all of:
(i) simple and efficient delivery of dsDNA donor into the cells;
(ii) producing single-stranded linear (ssIDNA) or single-stranded circular DNA (sscDNA) in cells in vivo from much stable double-stranded DNA (dsDNA) of the replicative donor vector;
(iii) tethering of ssIDNA to the target by covalent link of donor DNA with rep protein fused to cas9 or to bacteriophage coat protein (MS2 coat protein) in combination with stem-loop RNA structure (MS2 stem-loop) introduced into sgRNA; and
(iv) enhancing donor DNA accumulation near targeted locus providing excess of donor DNA for longer period of time.

Single-stranded donor DNA can be produced from linear dsDNA donor replicative vector with one origin of replication fused to 5′ end of donor DNA, or from linear or circular dsDNA replicative vector where donor DNA fragment is flanked by origins of replication on both 5′ and 3′ ends.

The invention also provides a nucleic acid encoding a third fusion protein comprising a recombination inducing domain and an RNA binding domain.

The recombination domain may be a protein or polypeptide that interacts with a target or donor nucleic acid molecule in order to catalyse modification of the nucleotide sequence of the target nucleic acid with reference to the nucleotide sequence of the donor nucleic acid molecule.

Modification of the target nucleic acid may be by way of insertion of all or a part of the sequence of the donor nucleic acid molecule or substitution of all or a part of the sequence of the donor nucleic acid molecule for a homologous section of the target nucleic acid molecule. In this way deletions, insertions, frameshift mutations and single nucleotide mutations may be achieved.

The recombination inducing event caused or mediated by the recombination inducing domain may be initiating or catalysing strand exchange between the target and donor nucleic acid molecules.

The recombination domain may be RecA from E. coli or a homologue thereof, Rad51 or a homologue thereof from a plant or another organism, or an annealase from such as bacteriophage A recombination protein beta (BET; Redβ) or a homologue thereof. Studies of phage lambda in vivo have indicated that bacteriophage A beta protein can catalyse steps that are central to both the strand annealing and strand invasion pathways of recombination. A homologous protein in this case may have functional or sequence homology, preferably functional homology.

Preferably the recombination domain is a trimer of RecA (SEQ ID NO: 13) or Rad51 (SEQ ID NO: 14) monomers. Most preferably the monomers are joined by peptide linkers. Use of a trimer of monomers for the recombination domain is advantageous because this allows binding of a turn of the nucleic acid helix in order to more efficiently initiate recombinase loading and strand exchange and hence HDR.

The invention also provides a nucleic acid encoding a fourth fusion protein comprising a domain comprising an inhibitor of the mismatch repair pathway; and an RNA binding domain.

MSH2 and MSH6 are proteins involved in base mismatch repair and the repair of short insertion/deletion loops. The MSH2 dominant-negative mutant (Sia et al., 2001, doi: 10.1128/MCB.21.23.8157-8167.2001) (SEQ ID NO: 15) competes with MSH2 binding to mismatches thus blocking the ability of the wild-type MSH2 protein to repair these mismatches. A dominant negative allele of MSH6 is also known and may be used in the same way as the dominant negative allele of MSH2 (Bowers et al., 1999, doi: 10.1074/jbc.274.23.16115) (SEQ ID NO: 16).

The invention further provides a nucleic acid encoding a fifth fusion protein comprising a domain comprising a Holliday junction resolvase and an RNA binding domain. The resolvase is suitably a bacteriophage T4 endonuclease VII (T4E7) (SEQ ID NO: 17), a bacteriophage T7 endonuclease I (Babon et al., 2003, doi: 10.1385/MB:23:1:73); CCE1 (SEQ ID NO: 18) a YDC2 resolvases from yeast (Kleff et al., 1992, PMCID:PMC556502; White et al., 1997, doi:10.1128/MCB.17.11.6465); a GEN1 resolvase from human (Ip et al., 2008, doi: 10.1038/nature07470) (SEQ ID NO: 19), or an AtGEN1 resolvase from Arabidopsis thaliana (SEQ ID NO: 20), (Bauknecht & Kobbe, 2014, doi: 10.1104/pp.114.237834).

The rearrangement and repair of DNA by homologous recombination involves the creation of Holliday junctions, which are cleaved by a class of junction-specific endonucleases to generate recombinant duplex DNA products.

The formation of DNA joint molecules is a transient process, which usually disrupted at an early stage by anti-recombinogenic helicases such as Srs2, Mph1 or RTEL1 (Gangloff et al., 1994, PMCID: PMC359378; Malkova et al., 2003, PMCID: PMC4493758: Prakash et al., 2009, doi: 10.1101/gad.1737809).

In somatic cells HDR is suppressed by low expression of resolvase and high activities of anti-recombinogenic helicases. The DNA helicase that translocates along single-stranded DNA in the 3′ to 5′ direction displaces annealed DNA fragments and removes Holliday junction intermediates from a crossover-producing repair pathway thereby reducing crossovers and HDR (Malkova et al., 2003, PMCID: PMC4493758).

In order to improve efficiency of HDR, timely delivery of resolvase to Holliday junctions, formed during donor DNA annealing, should be provided to fix recombination event and translate it into the modification at the target site.

The RNA binding domains of any of the first, third, fourth and fifth fusion proteins may bind to the RNA component of an RNA-guided endonuclease for use in transformation mediated by the RNA-guided endonuclease. Preferably an RNA component is a tracrRNA molecule or domain for use in transformation using the CRISPR-Cas9 system. Reference to a given domain comprising, say, a RNA binding domain includes the given domain both being and comprising that specified domain.

The invention also provides a method of transforming the genome of a human or animal cell comprising the steps of:
a. expressing an RNA-guided endonuclease in the cell or introducing the RNA-guided endonuclease into the cell;
b. expressing in the cell or introducing into the cell a sequence specific guide RNA to direct cleavage by the endonuclease domain to a specific locus;
c. expressing in the cell the nucleic acid encoding the first fusion protein or introducing the first fusion protein into the cell.

Thus the invention provides a system with multiple features that may be used separately or in concert. These features include one or more or all of:
a. Induction of dsDNA break using the sequence-specific endonuclease of the second fusion protein.
b. Amplification and delivery of donor nucleic acid molecule to within close proximity of the induced DNA break by associating the donor nucleic acid molecule with the origin-binding domain of the second fusion protein.
c. Suppression of religation of DNA breaks (i.e. blocking NHEJ), preferably, as noted above, by 5′ to 3′ resection of double-stranded DNA breaks in order to produce a substrate that is not suitable for NHEJ but is more suitable for HDR.
d. Delivery of recombinase to the induced dsDNA break.
e. Suppression of the mismatch repair pathway in the vicinity of the induced dsDNA breaks by providing an inhibitor of this pathway. As noted above this is preferably a fusion protein comprising a dominant negative suppressor protein of the mismatch repair system.
f. Resolution of Holiday junctions after invasion and annealing of donor DNA in the target site by delivery of the resolvase protein to DSB.

Features c, d, e and f are supplied to the HDR complex by their being provided in the form of the first, third, fourth and fifth fusion proteins, i.e. each comprises a domain that binds to an aptamer engineered to be part of the sgRNA that guides the endonuclease activity of the second fusion protein.

The first, third, fourth and fifth fusion proteins each comprises a domain that binds to an aptamer engineered to be part of the sgRNA that guides the endonuclease activity of an RNA-guided endonuclease. Therefore the first, third, fourth and fifth fusion proteins may be used in concert with an RNA-guided endonuclease other than the second fusion protein, such as Cas9 or Cpf1.

Feature (b) may also be provided comprising a domain that binds to an aptamer engineered to be part of the sgRNA that guides the endonuclease activity of an RNA-guided endonuclease.

One advantage flowing from use of any or all of the first, second, third, fourth and/or fifth fusion proteins of the invention is more reliable and efficient genetic modification.

A further advantage is that use of any or all of the first, second, third, fourth and/or fifth fusion proteins of the invention allows for insertion of longer DNA sequences at a locus or loci acted on by a sequence-guided endonuclease than has previously been reported.

The invention also provides a method of modifying the genome of a human or animal, or human or animal cell comprising:
a. expressing in the cell the nucleic acid encoding the second fusion protein or introducing the second fusion protein into the cell; and
b. expressing in the cell or introducing into the cell a donor nucleic acid molecule comprising an origin of replication.

As will be appreciated, the second fusion protein comprises an endonuclease domain and a binding domain for an origin of replication, wherein the binding domain suitably matches, e.g. binds to, the origin of replication of the donor nucleic acid.

Advantageously, the second fusion protein is capable of performing multiple functions. These functions include one or more of, or all of:
production of ssDNA of donor from dsDNA;
amplification of donor DNA;
tethering of donor DNA to the target; and
accumulation of donor DNA in close proximity to the target.

Particular advantage(s) are yielded by amplifying donor DNA and/or accumulating in close proximity to the target: accumulation of donor DNA near the locus of the DNA double-strand break promotes repair of the break by HDR. Providing a greater concentration of donor DNA and/or providing a greater local concentration of donor DNA near the target locus promotes HDR. Without wishing to be bound by theory, this is because the greater availability of a donor with a section homologous to the target means that the less accurate but quicker NHEJ pathway is not favoured under these conditions.

An animal in the context of the present disclosure may by any multicellular vertebrate or invertebrate animal. Suitably, the animal is a model organism used for biological, physiological or genetic research. Accordingly the animal may be selected from: mouse (Mus musculus), zebrafish (Danio rerio), fruit fly (Drosophila melanogaster), cat (Fells sylvestris catus), chicken (Gallus gallus), dog (Canis lupus familiaris), guinea pig (Cavia porcellus), rat (Rattus norvegicus) and nematode worm (Caenorhabditis elegans).

Suitably, the animal is a domesticated or farmed animal. Accordingly the animal may be selected from: goat (Capra aegagrus hircus), pig (Sus scrofa domesticus), sheep (Ovis aries), cattle (Bos taurus), cat (Fells catus), donkey (Equus africanus asinus), duck (Anas platyrhynchos domesticus), water buffalo, including “river buffalo” (Bubalus bubalis bubalis) and “swamp buffalo” (Bubalus bubalis carabenesis), Western honey bee (Apis mellifera), including subspecies Italian bee (A. mellifera ligustica), European dark bee (A. mellifera mellifera), Carniolan honey bee (A. mellifera carnica) and Caucasian honey bee (A. mellifera caucasia), Greek bee (A. mellifera cecropia), dromedary camel (Camelus dromedarius), horse (Equus ferus caballus), silkmoth (Bombyx mon), pigeon (Columba livia a), goose (Anser domesticus and Anser cygnoides domesticus), yak (Bos grunniens), bactrian camel (Camelus bactrianus), llama (Lama glama), alpaca (Vicugna pacos), guineafowl (Numida meleagris), ferret (Mustela putorius furo), turkey (Meleagris gallopavo) grass carp, silver carp, common carp, nile tilapia, bighead carp, catla (indian carp), crucian carp, atlantic salmon, roho labeo, milkfish, rainbow trout, wuchang bream, black carp, northern snakehead and amur catfish.

The donor nucleic acid molecule may comprise:
a. a donor nucleic acid sequence; and
b. origins of replication flanking nucleotide sequence of donor dsDNA molecule at both 5′ and 3′-ends, or
c. origin of replication flanking nucleotide sequence of donor dsDNA molecule at 5′ end and a replication terminator flanking nucleotide sequence at the 3′ end, or
d. origin of replication flanking nucleotide sequence of linear donor dsDNA molecule only at 5′-end.

The replication terminator may be a non-functioning origin of replication that is still capable of terminating replication when a replication fork reaches it. In a specific example, a circovirus (e.g. from porcine circovirus) origin of replication is nicked by the Rep protein at a particular location on a stem loop characteristic of the origin of replication. As long as the stem loop is present and correctly nicked then replication will be terminated at that location. Other sequence elements of the origin are not essential for termination and therefore can be omitted from the replication terminator in this example.

However, the nick at the replication terminator derived from such an origin of replication (in, for instance circoviruses) may still be competent for religation of the nicked stem loops at the active origin of replication and the downstream terminator/origin of replication. In this way a nucleic acid circle with an active origin of replication is provided and may be actively replicated by rolling circle replication or another mode of replication.

Rolling circle replication of the donor DNA acid molecule has the advantage of providing a large amount of donor DNA nucleic acid. Provision of a relatively large amount of donor nucleic acid molecule means that the probability of the successful transformation is raised.

The invention also provides a method of recovery of modified cells using replicon selection vector. In examples below this has been shown to be efficient.

Although modification in desirable locus of the cells can be introduced, recovery of modified clones from such cells is difficult due to competition between modified and non-modified cells. Recovery of modified clone from the population of modified and non-modified cells can be tedious and time-consuming.

The method provides specific replicon selection vector allowing selection for clones with desirable modification.

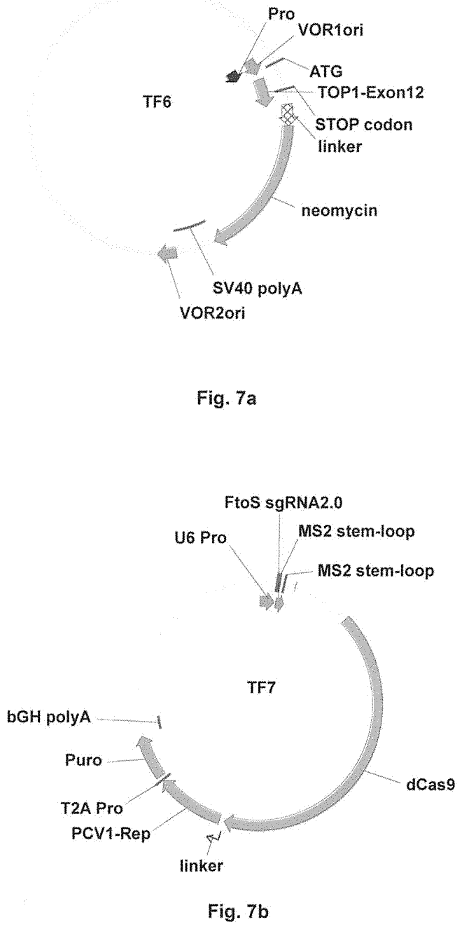

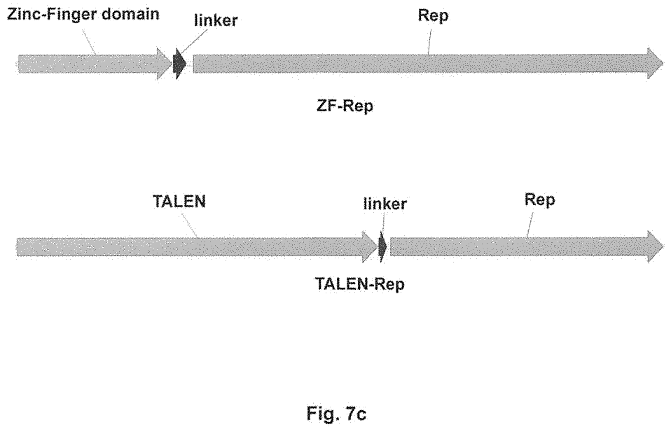

One example of the selection vector for introduction of knock out mutation in the cell and recovery of clones on selection media is presented in FIG. 7.

Accordingly, also provided by the invention is a selection vector comprising first and second viral origins of replication, wherein the first and second viral origins of replication are arranged to flank a donor DNA fragment; and the donor DNA fragment comprises a selectable marker gene that is fused out of frame.

The first and second viral origins of replication may be arranged to flank a DNA sequence comprising a promoter and a donor DNA fragment, and the donor DNA fragment may comprise a selectable marker gene that is out of frame with the promoter.

One such introduced selection vector comprises two PCV1 viral origins of replication flanking donor DNA fragment and selectable marker gene fused in translational frame. The viral origin of replication at 5′ end of the donor DNA contains native host-specific promoter with ATG translation codon, fused in translational frame with donor DNA fragment, linker, selectable marker gene (such as neomycin (SEQ ID NO: 21) or puromycin resistance genes (SEQ ID NO: 22)) terminator (such as SV40 polyA (SEQ ID NO: 23)), following by 3′ end viral origin of replication. All sequences introduced after ATG codon, represent one translational unit, generating resistance to antibiotic, e.g. neomycin or puromycin antibiotic.

In order to introduce a knock out mutation into specific gene, a stop codon should be introduced in the donor DNA fragment. As the stop codon is introduced into donor fragment in front of selectable marker gene, no antibiotic resistance generated by the selection donor vector will be observed due to premature termination of translational unit on selection vector.

Recombination of the donor DNA fragment with the target transfers stop codon to the target sequence, while the DNA fragment without stop codon from the target replaces donor fragment in the selection donor vector. As result, the translational unit on the donor vector is restored, and the replicon is amplified, allowing selection on antibiotic supplemented medium. The cells where translational unit of the donor vector is restored by exchange between donor and target DNA strands during recombination process are resistant to antibiotic selection, and clones can be recovered from such cells on selection medium.

As Rep expression may be provided in cells transiently, the antibiotic selection vector DNA will be degraded as soon as Rep protein is expired after 7-8 days and the modified cloned can be recovered on selection medium. Subsequent propagation of modified clones should be performed on antibiotic free medium.

The invention also provides a method of efficient complementation of common single-gene disorders using replicon vector.

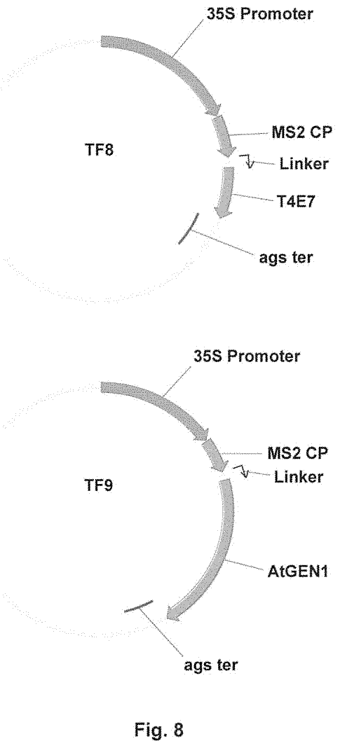

The replicative vector (replicon) described in this invention can be directly utilised for human gene therapy to generate expression of correct copy of genes in the mutated background of the host. For this purpose the vector, containing viral origins of replication of torque teno virus (TTV) at both 5′ and 3′ ends, flanking the cassette with correct sequence of desirable gene under native gene promoter and polyA, can be used (FIG. 8).

Torque teno virus (TTV) is a circular, single-stranded DNA virus that chronically infects healthy individuals of all ages worldwide. TTVs have a single stranded circular DNA of approximately 3.8 kb and are extraordinarily diverse, spanning five groups including SAN BAN and SEN viruses. TTVs are ubiquitous in >90% of adults with relatively uniform distribution worldwide, but no human pathogenicity of TTV has been fully established. TTV DNA was detected in different organs and tissues such as bone marrow, lymph nodes, muscles, thyroid, lungs, spleen, pancreas, kidneys, cerebrospinal fluid, nervous tissue (DOI: 10.1007/s00705-015-2363-9). Such widespread organ distribution allows performing gene therapy of wide spectrum of the inherited diseases.

As TTV Rep gene is already present in the body of most patients, delivery of replicon with correct gene can complement for the mutated copy of the gene in the host by replicating in different organs and providing expression of correct protein from the replicon. The sequence of TTV origin of replication has already been described (SEQ ID NO: 24) and the formation of additional replication-competent subviral molecules using this viral origin of replication has been demonstrated in vitro (DOI: 10.1128/JVI.02472-10),

Thus the combination of replicon vector, containing correct copy of therapeutic gene, with viral Rep genes provided from native TTV virus of infected patient can complement for wide range of gene mutations responsible for different inherited diseases. In our experiments a replicon of up to 20 kb was engineered providing the possibility of expression for long gene sequences.

Accordingly, the invention provides a method of transforming the genome of a human or animal cell comprising the step of introducing a donor construct comprising a donor DNA molecule into the cell, wherein the donor DNA molecule comprises (a) a sequence of nucleic acids homologous to the intended target, or a sequence for complementation of a mutated copy of a target, a promoter, a correct copy of the gene and a 3′ UTR, and wherein the construct comprises (b) one or more viral origin(s) of replication flanking the donor DNA molecule. One donor DNA molecule comprises a sequence of nucleic acids homologous to the intended target, and wherein the construct comprises (b) one or more viral origin(s) of replication flanking the sequence. A further suitable donor DNA molecule comprises a sequence for complementation of a mutated copy of an intended target and viral origins of replication flanking the complementation sequence and is for use in therapy. The viral origin(s) of replication is preferably from a virus infection by which is substantially asymptomatic, especially asymptomatic in humans. The virus may be commensal with respect to animals, especially humans. An example is torque teno virus. The donor may further comprise a 3′ UTR. The invention further provides the donor DNA for use in human therapy. The donor may be used in humans infected by the virus, as this provides the requisite replicase for therapy activity in vivo. An option, for patients not yet infected by the virus, is to infect the patient(s) with the virus prior to or at the same time as administration (e.g. by injection) of the donor.

Therapy suitably includes preparation of replicon with the desirable gene based on the viral origin of replication, e.g. TTV origin of replication, generation of replication-competent subviral molecules in vitro, intravenous injection of the replication-competent DNA molecules into the host.

Accordingly, the method may also comprise the steps of:
a. expressing in the cell one or more nucleic acids encoding the first, third, fourth and fifth fusion proteins; or
b. introducing into the cell one or more of the first, third, fourth and fifth fusion proteins.

Treatment of the common single-gene disorders, for example cystic fibrosis, hemochromatosis, Tay-Sachs, sickle cell anaemia, fragile X syndrome, muscular dystrophy and Huntington disease may be performed using this invention.

The methods described herein may comprise introducing a double strand break into the genome in the presence of an exogenous donor nucleic acid molecule comprising a donor nucleic acid sequence as a template for modifying the genome or as an exogenous sequence to be integrated into the genome and a DNA repair mechanism modifies the genome via homology-directed repair (HDR).

The method may further comprise the step or effect of suppressing non-homologous end joining (NHEJ) repair of a DNA double-strand break to promote repair of the break by HDR by expressing in the cell a nucleic acid encoding the first fusion protein or introducing the first fusion protein into the cell.

The methods described herein may comprise introducing a double strand break into the genome in the presence of an exogenous donor nucleic acid molecule comprising a donor nucleic acid sequence as a template for modifying the genome or as an exogenous sequence to be integrated into the genome and a DNA repair mechanism modifies the genome via homology-directed repair (HDR) the method comprising:
suppressing non-homologous end joining (NHEJ) repair of the break to promote repair by HDR by expressing in the cell a nucleic acid encoding the first fusion protein or introducing the first fusion protein into the cell.

The first protein may be also expressed without fusion to an RNA aptamer binding protein under a constitutive promoter or as mRNA, however efficiency of NHEJ suppression is reduced.

The method may further comprise the steps of:
a. expressing in the cell one or more nucleic acids encoding the second, third, fourth and fifth fusion proteins; or
b. introducing into the cell one or more of the second, third, fourth and fifth fusion proteins.

The method may further comprise the steps of:
a. expressing in the cell or introducing into the cell a sequence specific guide RNA to direct cleavage by the endonuclease domain to a specific locus; and
b. expressing in the cell one or more nucleic acids of the first, third, fourth and fifth fusion proteins or introducing one or more of the first, third, fourth and fifth fusion proteins into the cell.

The method may further comprise the step of expressing in the cell two or more nucleic acids encoding the first, third, fourth and fifth fusion proteins or introducing into the cell two or more of the first, third, fourth and fifth fusion proteins, wherein the RNA binding protein domains of the respective fusion proteins bind to different RNA sequences.

In this way the first fusion protein may be using in concert with the second, third, fourth and fifth fusion proteins for transformation of a non-animal cell or organism in concert with an RNA-guided endonuclease.

Expression of the first, second, third, fourth and fifth fusion proteins during a method of modifying the genome as described herein may be via inducible and/or transient expression.

Various methods for introducing nucleic acids encoding the fusion proteins and nucleic acids of the invention are envisaged these include electroporation and infiltration in order to introduce proteins, DNA and/or RNA. Also envisaged is the use of delivery systems, including liposomes or lipid nanoparticles (LNP), for directly introducing proteins, DNA and/or RNA, preferably by encapsulation of the proteins, DNA and/or RNA therein.

The invention further provides a first fusion protein comprising a 5′ to 3′ DNA exonuclease domain with or without an RNA binding domain.

The invention also provides a second fusion protein comprising an endonuclease and a component of the replication initiation complex or replication complex.

The invention also provides a third fusion protein comprising a recombination inducing domain and an RNA binding domain

The invention further provides a fourth fusion protein comprising a domain comprising an inhibitor of the mismatch repair pathway and an RNA binding domain.

The invention further provides a fifth fusion protein comprising a domain for Holiday junction resolution with or without an RNA binding domain.

The invention further provides for use of the first fusion protein, or a nucleic acid encoding the fusion protein in transformation of a human or animal cell, or human or animal cell line using an RNA-guided endonuclease.

The invention further provides for use of the second fusion protein, or a nucleic acid encoding the second fusion protein in transformation of a non-animal organism or cell.

The invention also provides for use of the first fusion protein, or a nucleic acid encoding the first fusion protein in concert with the second, third, fourth and fifth fusion proteins in transforming a non-animal organism or cell using an RNA-guided endonuclease.

The invention further provides vectors comprising the nucleic acids of the invention. Such vectors may be suitable for modification in vitro or in vivo.

Vectors of the invention capable of expressing products encoded on nucleotides of the invention may also be suitable for expression in a host cell or cell-free system. Suitably the host cell may be a cultured plant cell, yeast cell or bacterial cell, e.g. Escherichia coli. Compositions and products of the invention may be obtained by methods comprising expressing such encoded products in a suitable host cell or cell-free system.

The invention also provides uses of the methods, reagents and compositions disclosed herein for introducing desirable traits to non-animal organisms or ameliorating or removing non-desirable traits in these organisms. Accordingly, the invention also provides non-animal transgenic organisms, transgenic cells thereof and transgenic non-animal cell lines. Organisms which include a transgenic cell according to the invention are also provided.

The invention further provides methods of treating disease or other conditions of non-animal organisms or cells by utilising the methods, reagents and compositions disclosed herein.

The invention also provides the methods, reagents and compositions disclosed herein for use in the treatment of disease or humans or animals.

The invention also provides uses of the methods, reagents and compositions disclosed herein for introducing desirable genetic characteristics to humans or animals or ameliorating or for removing non-desirable genetic characteristics in humans or animals.

The invention further provides uses of the methods, reagents and compositions disclosed herein for introducing desirable heritable characteristics to non-human animals for ameliorating or for removing non-desirable inherited characteristics in these animals.

Accordingly, the invention also provides non-human transgenic animals, transgenic cells thereof and transgenic human or animal cell lines. Animals which include a transgenic cell according to the invention are also provided.

The invention further provides methods of treating disease or other conditions of humans or animals by utilising the methods, reagents and compositions disclosed herein.

The invention also provides uses of the methods, reagents and compositions disclosed herein for introducing desirable genetic characteristics to human or animal embryonic stem cells and/or stem cell lines or for ameliorating or removing non-desirable genetic characteristics in these stem cells and/or stem cell lines.

The invention further provides uses of the methods, reagents and compositions disclosed herein for therapeutic or diagnostic purposes applied to a human embryo and which are useful to it.

The invention is now illustrated in specific embodiments with reference to the accompanying drawings in which:

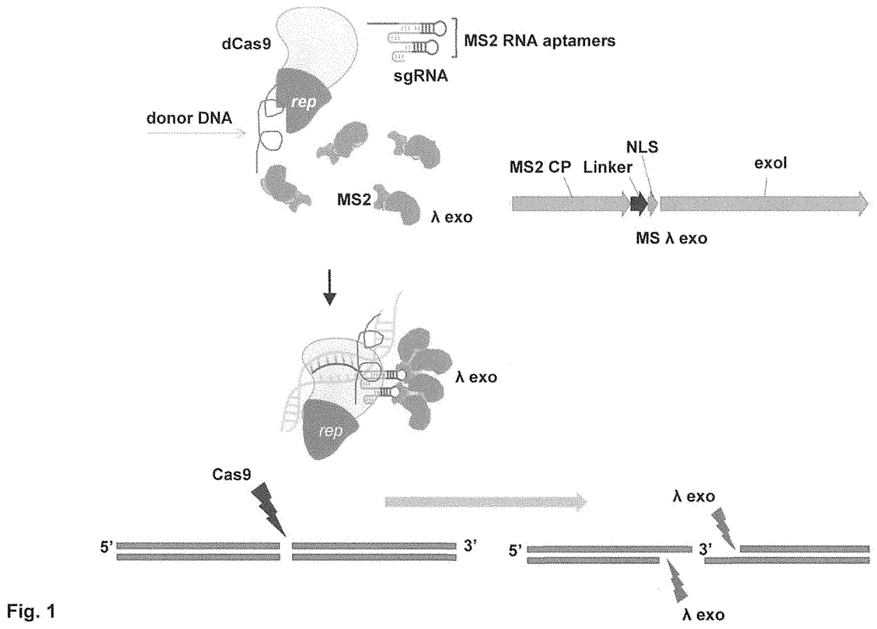

FIG. 1 shows a schematic representation of inducing a DNA double strand break with Cas9 protein (Cas9) and resecting the DNA DSB with λ-exo. The MS2-λ-exo fusion protein is engineered to bind to the single guide RNA (sgRNA) via aptamer loops on the sgRNA that bind to the MS2 domain. (SEQ ID NO: 25).

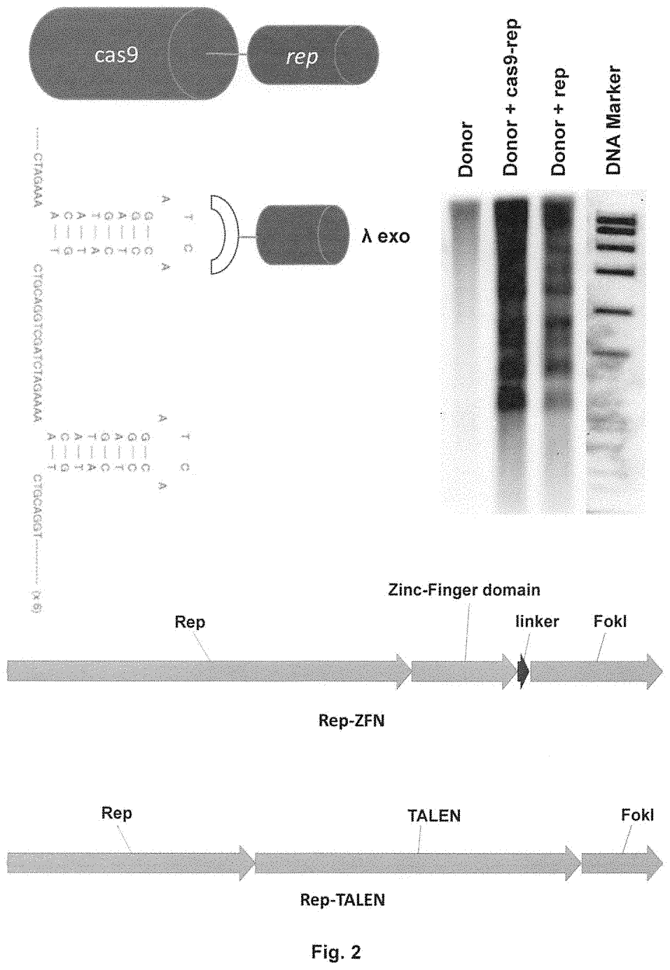

FIG. 2 shows a schematic representation of a cas9-PCV1 Rep (virus replication associated protein) fusion protein (SEQ ID NO: 11) and an MS2-λ-exo-fusion protein showing its binding to an aptamer loop. Also shown is an electrophoresis gel demonstrating the activity of the cas9-PCV1 Rep fusion protein. The Rep protein may instead be fused with zinc-finger or TALEN nucleases to yield similar activities to the cas9-Rep fusion.

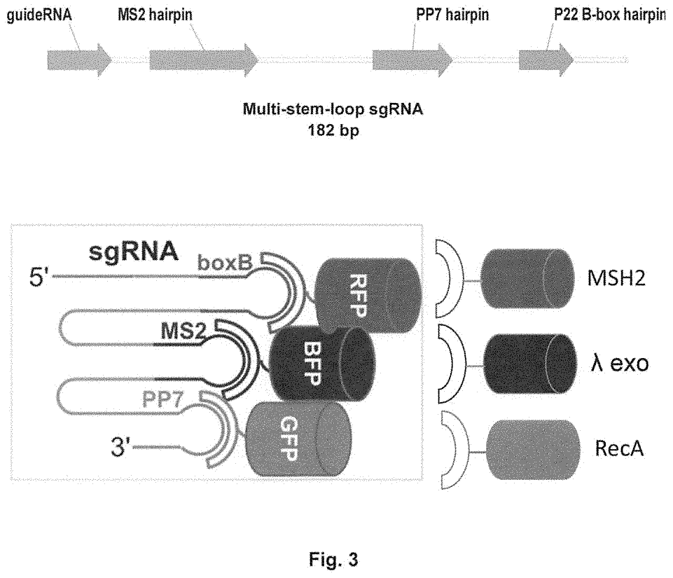

FIG. 3 shows the design of a multi stem-loop sgRNA with hairpins from different bacteriophages (MS2, PP7 and P22 bacteriophages; (SEQ ID NOs: 1, 3, 5 and 7-8).

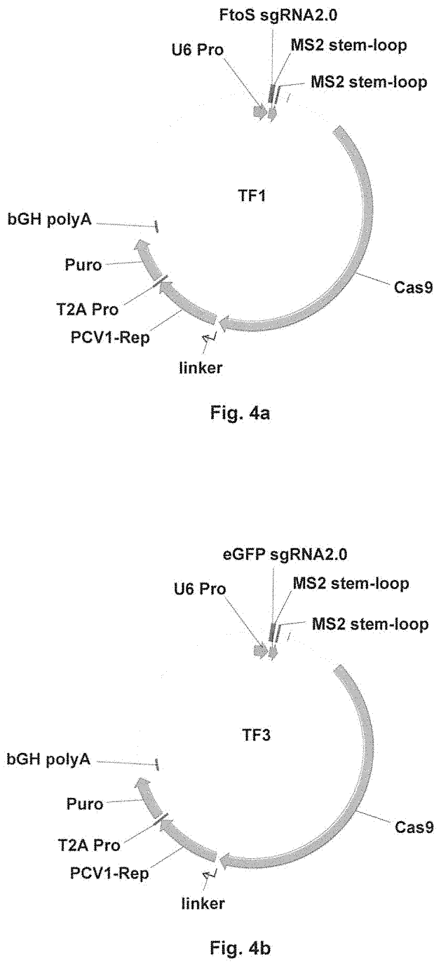

FIG. 4 shows the vectors for topoisomerase I gene modification in human HEK293 cells, vectors containing cas9-PCV1 Rep fusion and sgRNA2.0 for introduction of (4a) mutation into topoisomerase gene (TF1) and (4b) for precise insertion of eGFP into topoisomerase I locus (TF3).

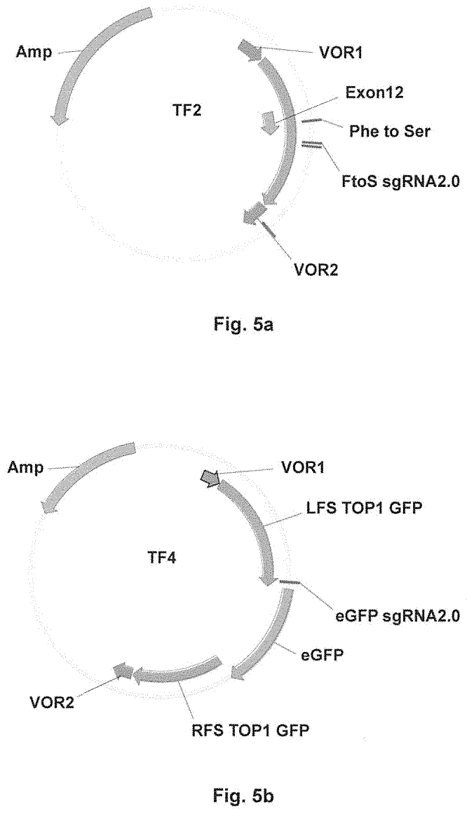

FIG. 5 shows (5a) a donor vector (TF2) containing a mutated topoisomerase I gene fragment for generation of resistance to antibiotic camptothecin (SEQ ID NO: 26), and (5b) a donor vector (TF4) comprising a cassette designed for insertion of eGFP into the topoisomerase I locus (SEQ ID NO: 27). POR1 and POR2 (SEQ ID NO: 12) are viral origins of replication from pig circovirus 1 (PCV1)

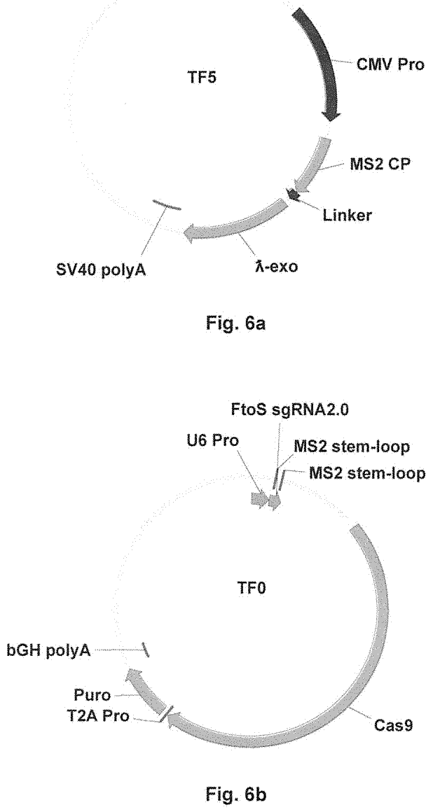

FIG. 6 shows (6a) vector containing a cassette for translational fusion of the MS2 coat protein (SEQ ID NO: 2) and λ-exo (SEQ ID NO: 9) to generate targeting of the λ-exo protein to double-stranded DNA breaks, (6b) control (TFO) vector containing cas9 (SEQ ID NO: 28) with sgRNA for introduction of camptothecin resistance into the topoisomerase I locus.

FIG. 7 shows (7a) cassette of replicon selection vector containing two PCV1 viral origins of replication with host-specific promoter, donor of human topoisomerase I with introduced stop codon, linker fused to selectable gene neomycin and SV40 polyA. The donor fragment is fused in frame with selection gene, thus representing one translational unit; (7b) vector with “dead” cas9 gene (dCas9), where nuclease activity centres were mutagenized (TF7) for introduction of camptothecin resistance into the topoisomerase I locus (7c). In alternative embodiments, alternative DNA binding domains such as zinc-finger or TALEN domains may be fused to Rep and utilised for this approach.

FIG. 8 shows constructs containing T4 endonuclease VII (T4E7) resolvase from bacteriophage T4 (TF8) and AtGEN1 resolvase from Arabidopsis (TF9) fused to MS2 coat protein to target them with sgRNA.

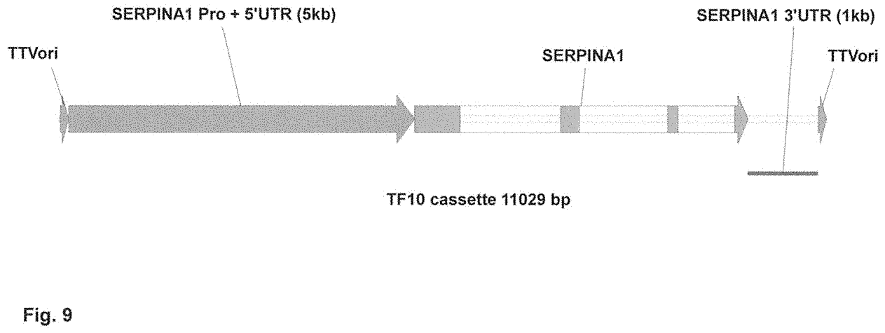

FIG. 9 shows replicon vector for complementation of common single-gene disorder such as alpha-1 antitrypsin deficiency using expression of correct SERPINA1 gene (SEQ ID NO: 33) from replicon vector in different organs and tissues of the patient.

### EXAMPLE 1

### Gene Editing of Topoisomerase I in Human Cells

To assess efficiency of gene targeting in human cells a set of constructs was prepared for targeting topoisomerase I.

Transfections were carried out by calcium phosphate transfection (see Calcium phosphate-mediated transfection of eukaryotic cells, Nature Methods 2005, volume 2, pages 319-320 and kits derived therefrom as available from ThermoFisher Scientific or Merck) but may also be carried out by electroporation (see method below).

A mutant topoisomerase I gene fragment for generation of resistance to antibiotic camptothecin (SEQ ID NO: 26) was introduced into human HEK293 cells (topI donor-SKM; FIG. 4a). Co-transfection of human HEK293 cells was performed using TF1 vector (containing FtoS sgRNA2.0 (SEQ ID NO: 29) and cas9-PCV1 Rep) (FIG. 4a), TF2 vector (containing modified donor DNA) (FIG. 5a) and TF5 vector (containing MS2-λ-exo) (FIG. 6a). The clones recovered after transfection were analysed by PCR and sequencing (Table 1). Positive for mutation clones have confirmed resistance to camptothecin antibiotic.

TABLE 1

| | | | Percentage of |
|---|---|---|---|
| Experiment in human | Heterozygous | Homozygous | homozygous |
| HEK293 cells | clones | clones | clones |
| (i) Donor vector POR12 | 2 out of 47 | 0 out of 47 | 0% |
| TOP1 FtoS sgRNA2.0 | | | |
| cas9 | | | |
| (ii) Donor vector POR12 | 4 out of 47 | 3 out of 47 | 6.3% |
| TOP1 FtoS sgRNA2.0 | | | |
| cas9-PCV1-Rep | | | |
| (iii) Donor vector POR12 | 6 out of 47 | 4 out of 47 | 8.5% |
| TOP1 FtoS sgRNA2.0 | | | |
| cas9-PCV1-Rep | | | |
| MS2- λ-exo | | | |

These results demonstrate that gene editing mediated by the Cas9-PCV1Rep fusion (SEQ ID NO: 11) (experiment (ii)) is significantly more efficient than for the control experiment using cas9 alone.

These results also demonstrate that gene editing mediated by the Cas9-PCV1 Rep fusion and an MS2-λ-exo fusion protein designed to bind to the Cas9-sgRNA complex (experiment (iii)) is yet more efficient than either the control experiment using cas9 alone or experiment (ii) using the Cas9-PCV1Rep fusion alone.

### EXAMPLE 2

Insertion of eGFP into the Topoisomerase I Locus of Human Cells

Human cells carrying the mutated topoisomerase I gene generated by the method described above were then co-transformed with donor construct TF4 (topl-eGFP donor-SKM) (SEQ ID NO: 27) (FIG. 5b) and constructs expressing (i) Cas9 (TFO) (FIG. 6b) (ii) Cas9-PCV1Rep fusion or (iii) Cas9-PCV1Rep fusion (TF3) (FIG. 4b) and an MS2-λ-exo fusion protein (TF5) designed to bind to the sgRNA that is in turn bound to the cas9 (FIG. 6a).

The cells subsequently generated were assessed for eGFP activity; eGFP activity indicating successful transformation in vivo using the gene targeting system. The results of these experiments are set out in Table 2.

TABLE 2

| | | | |
|---|---|---|---|
| Experiment in human | | | Percentage of |
| HEK293 cells with | Heterozygous | Homozygous | homozygous |
| eGFP (717 bp) | clones | clones | clones |

| | | |
|---|---|---|
| (i) Donor vector POR12 | 0 out of 47 | 0% |
| TOP1 eGFP sgRNA2.0 | | |
| cas9 | | |
| (ii) Donor vector POR12 | 0 out of 47 | 0% |
| TOP1 eGFP sgRNA2.0 | | |
| cas9-PCV1-Rep | | |
| (iii) Donor vector POR12 | 3 out of 47 | 6.3% |
| TOP1 eGFP sgRNA2.0 | | |
| cas9-PCV1-Rep | | |
| MS2- λ-exo | | |

The transformation being carried out in this instance is a relatively large insertion of 717 bp. As noted above, hereto now it has not been possible to insert longer sequences of nucleotides into a locus targeted by RNA-directed mutagenesis.

These results also demonstrate that gene editing mediated by the Cas9-PCV1 Rep fusion in concert with an MS2-λ-exo fusion protein designed to bind to the sgRNA complex (experiment (iii)) allows the efficient insertion of longer nucleotide sequences into the targeted locus. As is also demonstrated such an insertion is not achieved by using cas9 alone or the Cas9-PCV1Rep fusion alone.

### EXAMPLE 3

### Selection for Knock Out Topoisomerase I Mutant in HEK293 Cells Using Replicon Selection Vector.

In order to accelerate generation of desirable modification in the cells a replicon selection vector has been developed (FIG. 7a). The selection approach relies on three factors:
(i) strand exchange between donor and target DNA, so that modification in the donor vector due to such exchange generates a functional translational unit connected to the selection gene.
(ii) transient amplification of the modified donor using PCV1-Rep.
(iii) selection of the transfected cells on specific antibiotic for 5-10 days.

The donor DNA replicon selection vector was designed to introduce stop codon into the exon 12 of topoisomerase I (SEQ ID NO: 31). Co-transformation of the HEK293 cells was performed with three vectors: TF1 (FtoS sgRNA2.0-cas9-PCV1 Rep) (FIG. 5a), TF5 (MS2-λ-exo) (FIG. 6a) and replicon selection vector TF6 (FIG. 7a). The colonies were recovered on neomycin supplemented media after 7 days of selection. Generation of knock out mutant was confirmed by PCR analysis and sequencing.

### EXAMPLE 4

Introduction of Mutation into the Topoisomerase I Locus without Generation of DSB.

Introduction of mutation into the desirable locus without DSB would be more preferable compare to cas9 nuclease-mediated HDR, as the risk of “off target” events is still considerably high.

We prepared TF7 vector with mutated version of cas9-Rep (SEQ ID NO: 32) (FIG. 7b), where both nuclease activities sites were eliminated resulting in so-called “dead” cas9 nuclease (dCas9-Rep). Although nuclease activities were eliminated, dCas9-Rep still binds to sgRNA and recognises the target. As Rep gene is fused to dCas9, the donor DNA molecule covalently linked to Rep is still tethered to the target and can be annealing with target forming Holliday junctions. Such annealing of donor DNA with target and formation of Holliday junction are suppressed by endogenous helicases. In order to facilitate rapid resolution of Holliday junctions at the target site after annealing of donor DNA, we have co-delivered TF8 vector with bacteriophage T4 exonuclease VII (T4E7) or TF9 vector with Arabidopsis AtGEN1 resolvase fused to MS2 coat protein) to tether it to target site using MS2 stem-loops integrated into sgRNA (FIG. 8). The replicon donor selection vector TF6 was designed to introduce stop codon into exon12 of human topoisomerase I as indicated in example 3. Both bacteriophage and Arabidopsis resolvases have facilitated recovery of mutated cells in combination with replicon donor selection vector.

### EXAMPLE 5

### Complementation of Mutated Human SERPINA1 Locus Using Replicon-Based Vector.

A replicon vector (TF10) for complementation of mutated SERPINA1 locus responsible for alpha-1 antitrypsin deficiency was prepared which can be utilised for treatment of patients with this single gene disorder (FIG. 9). The vector comprises two torque teno virus origins of replication at 5′ and 3′ ends of donor cassette, containing SERPINA1 native 5′ untranslated region (UTR) sequence with promoter, correct copy of SERPINA1 gene followed by native 3′ UTR of the locus (SEQ ID NO: 33).

Such vector may be applied to patient who has been confirmed to be positive for presence of torque teno virus (TTV). The virus provides a source of native Rep protein, which can amplify replicon vector driving expression of correct gene copy in different organs of the patient.

### Transformation Method—Electroporation

This protocol was adapted from “DNA transfection by electroporation” in Molecular Cloning: A Laboratory Manual (eds. Sambrook, J. & Russell, D. W.) 16.33-16.36 (Cold Spring Harbor Laboratory Press, Cold Spring Harbor, N.Y., 2001; http://www.cshIpress.com/link/molclon3 htm).

### Preparation of the Cells

1. Collect the cells to be transfected from cultures in the mid- to late-logarithmic phase of growth. Use either a rubber policeman or trypsin to release adherent cells. Centrifuge at 500 g at 4° C. for 5 min.
2. Resuspend the cell pellet in 0.5× volume of the original growth medium and measure the cell number using a hemocytometer.
3. Collect the cells by centrifugation, as described in Step 1 and resuspend them in growth medium or phosphate-buffered saline (PBS; 137 mM NaCl, 2.7 mM KCl, 10 mM Na₂HPO₄ and 2 mM KH₂PO₄) at 15-25° C. at a concentration of 2.5×10⁶ to 2.5×10⁷ cells/ml.
4. Transfer 400-μl aliquots of the cell suspension (10⁶-10⁷ cells) into as many labeled electroporation cuvettes as needed. Place the loaded cuvettes on ice.
5. Set the parameters on the electroporation device. (A typical capacitance value is 1,050 μF.) Voltages range from 200 to 350 V, depending on the cell line, but generally average 260 V. Use an infinite internal resistance value. Discharge a blank cuvette containing PBS at least twice before electroporating cells.

### Introduction of the DNA

6. Add 10-30 μg of plasmid DNA in a volume of up to 40 μl to each cuvette containing cells. (Some investigators add carrier DNA, for example, salmon sperm DNA, to bring the total amount of DNA to 120 μg.) Gently mix the cells and DNA by pipetting the solution up and down. Proceed to Step 7 without delay.

Do not introduce air bubbles into the suspension during the mixing step.
7. Immediately transfer the cuvette to the electroporator and discharge the device. After 1-2 min, remove the cuvette, place it on ice and proceed immediately to the next step.
8. Transfer the electroporated cells to a 35-mm culture dish using a micropipettor equipped with a sterile tip. Rinse out the cuvette with a fresh aliquot of growth medium and add the washings to the culture dish. Transfer the dish to a humidified incubator at 37° C. with an atmosphere of 5-7% CO₂.
9. Repeat Steps 6-8 until all of the DNA cell samples have been treated. Recording the actual pulse time for each cuvette will facilitate comparisons between experiments.
10. If the objective is stable transformation of the cells, proceed directly to Step 11. For transient expression, examine the cells 24-96 h after electroporation using an appropriate assay.
11. To isolate stable transfectants, incubate for 48-72 h in complete medium, trypsinize the cells and replete them in the appropriate selective medium. Change the selective medium every 2-4 d for 2-3 weeks to remove the debris of dead cells and to allow colonies of resistant cells to grow. Thereafter, clone individual colonies and propagate for the appropriate assay.

Nucleotide Sequences
MS2-derived stem-loop for binding
(SEQ ID NO: 1)
ggccaacatgaggatcacccatgtctgcagggcc
MS2 coat protein
(SEQ ID NO: 2)
atggcttcaaactttactcagttcgtgctcgtggacaatggtggga
caggggatgtgacagtggctccttctaatttcgctaatggggtggc
agagtggatcagctccaactcacggagccaggcctacaaggtgaca
tgcagcgtcaggcagtctagtgcccagaagagaaagtataccatca
aggtggaggtccccaaagtggctacccagacagtgggcggagtcga
actgcctgtcgccgcttggagatcctacctgaacatggaactcact
atcccaattttcgctaccaattctgactgtgaactcatcgtgaagg
caatgcaggggctcctcaaagacggcaatcctatcccttccgccat
cgccgctaactcaggca
PP7-derived stem-loop for binding
(SEQ ID NO: 3)
taaggagtttatatggaaaccctta
PP7 bacteriophage coat protein
(SEQ ID NO: 4)
atgtccaaaaccatcgttctttcggtcggcgaggctactcgcactc
tgactgagatccagtccaccgcagaccgtcagatcttcgaagagaa
ggtcgggcctctggtgggtcggctgcgcctcacggcttcgctccgt
caaaacggagccaagaccgcgtatcgagtcaacctaaaactggatc
aggcggacgtcgttgattgctccaccagcgtctgcggcgagcttcc
gaaagtgcgctacactcaggtatggtcgcacgacgtgacaatcgtt
gcgaatagcaccgaggcctcgcgcaaatcgttgtacgatttgacca
agtccctcgtcgcgacctcgcaggtcgaagatcttgtcgtcaacct
tgtgccgctgggccgttaa
P22-derived stem-loop for binding bacteriophage
B-box
(SEQ ID NO: 5)
accgccgacaacgcggt
P22 bacteriophage coat protein
(SEQ ID NO: 6)
atgacggttatcacctacgggaagtcaacgtttgcaggcaatgcta
aaactcgccgtcatgagcggcgcagaaagctagccatagagcgcga
caccatctgcaatatcatcgattcaatttttggctgcgatgctcct
gatgcttctcaggaagttaaagccaaaagaattgaccgtgtcacca
aagccatttcgcttgccggaacgcgtcagaaggaagttgaaggagg
atctgtacttcttccaggcgtagcactttacgcggctggtcatcgt
aagagcaaacaaataacagcgaggtaa
sgRNA2.0 with MS2 hairpins for modification of
topoisomerase I gene
(SEQ ID NO: 7)
gatttacaacaaaacccagcgttttagagctaggccaacatgagga
tcacccatgtctgcagggcctagcaagttaaaataaggctagtccg
ttatcaacttggccaacatgaggatcacccatgtctgcagggccaa
gtggcaccgagtcggtgcttttttttttt
sgRNA comprising multiple stem-loops
(SEQ ID NO: 8)
gttttagagctaggccaacatgaggatcacccatgtctgcagggcc
tagcaagttaaaataaggctagtccgttatcaactttaaggagttt
atatggaaacccttaaagtggcaccgagtcggtgctaccgccgaca
acgcggttttttttttt
λ-exo gene from bacteriophage lambda
(SEQ ID NO: 9)
atgacaccggacattatcctgcagcgtaccgggatcgatgtgagag
ctgtcgaacagggggatgatgcgtggcacaaattacggctcggcgt
catcaccgcttcagaagttcacaacgtgatagcaaaaccccgctcc
ggaaagaagtggcctgacatgaaaatgtcctacttccacaccctgc
ttgctgaggtttgcaccggtgtggctccggaagttaacgctaaagc
actggcctggggaaaacagtacgagaacgacgccagaaccctgttt
gaattcacttccggcgtgaatgttactgaatccccgatcatctatc
gcgacgaaagtatgcgtaccgcctgctctcccgatggtttatgcag
tgacggcaacggccttgaactgaaatgcccgtttacctcccgggat
ttcatgaagttccggctcggtggtttcgaggccataaagtcagctt
acatggcccaggtgcagtacagcatgtgggtgacgcgaaaaaatgc
ctggtactttgccaactatgacccgcgtatgaagcgtgaaggcctg
cattatgtcgtgattgagcgggatgaaaagtacatggcgagttttg
acgagatcgtgccggagttcatcgaaaaaatggacgaggcactggc
tgaaattggttttgtatttggggagcaatggcgatga
cas9 gene
(SEQ ID NO: 10)
atggactataaggaccacgacggagactacaaggatcatgatattg
attacaaagacgatgacgataagatggccccaaagaagaagcggaa
ggtcggtatccacggagtcccagcagccgacaagaagtacagcatc
ggcctggacatcggcaccaactctgtgggctgggccgtgatcaccg
acgagtacaaggtgcccagcaagaaattcaaggtgctgggcaacac
cgaccggcacagcatcaagaagaacctgatcggagccctgctgttc
gacagcggcgaaacagccgaggccacccggctgaagagaaccgcca
gaagaagatacaccagacggaagaaccggatctgctatctgcaaga
gatcttcagcaacgagatggccaaggtggacgacagcttcttccac
agactggaagagtccttcctggtggaagaggataagaagcacgagc
ggcaccccatcttcggcaacatcgtggacgaggtggcctaccacga
gaagtaccccaccatctaccacctgagaaagaaactggtggacagc
accgacaaggccgacctgcggctgatctatctggccctggcccaca
tgatcaagttccggggccacttcctgatcgagggcgacctgaaccc
cgacaacagcgacgtggacaagctgttcatccagctggtgcagacc
tacaaccagctgttcgaggaaaaccccatcaacgccagcggcgtgg
acgccaaggccatcctgtctgccagactgagcaagagcagacggct
ggaaaatctgatcgcccagctgcccggcgagaagaagaatggcctg
ttcggaaacctgattgccctgagcctgggcctgacccccaacttca
agagcaacttcgacctggccgaggatgccaaactgcagctgagcaa
ggacacctacgacgacgacctggacaacctgctggcccagatcggc
gaccagtacgccgacctgtttctggccgccaagaacctgtccgacg
ccatcctgctgagcgacatcctgagagtgaacaccgagatcaccaa
ggcccccctgagcgcctctatgatcaagagatacgacgagcaccac
caggacctgaccctgctgaaagctctcgtgcggcagcagctgcctg
agaagtacaaagagattttcttcgaccagagcaagaacggctacgc
cggctacattgacggcggagccagccaggaagagttctacaagttc
atcaagcccatcctggaaaagatggacggcaccgaggaactgctcg
tgaagctgaacagagaggacctgctgcggaagcagcggaccttcga
caacggcagcatcccccaccagatccacctgggagagctgcacgcc
attctgcggcggcaggaagatttttacccattcctgaaggacaacc
gggaaaagatcgagaagatcctgaccttccgcatcccctactacgt
gggccctctggccaggggaaacagcagattcgcctggatgaccaga
aagagcgaggaaaccatcaccccctggaacttcgaggaagtggtgg
acaagggcgcttccgcccagagcttcatcgagcggatgaccaactt
cgataagaacctgcccaacgagaaggtgctgcccaagcacagcctg
ctgtacgagtacttcaccgtgtataacgagctgaccaaagtgaaat
acgtgaccgagggaatgagaaagcccgccttcctgagcggcgagca
gaaaaaggccatcgtggacctgctgttcaagaccaaccggaaagtg
accgtgaagcagctgaaagaggactacttcaagaaaatcgagtgct
tcgactccgtggaaatctccggcgtggaagatcggttcaacgcctc
cctgggcacataccacgatctgctgaaaattatcaaggacaaggac
ttcctggacaatgaggaaaacgaggacattctggaagatatcgtgc
tgaccctgacactgtttgaggacagagagatgatcgaggaacggct
gaaaacctatgcccacctgttcgacgacaaagtgatgaagcagctg
aagcggcggagatacaccggctggggcaggctgagccggaagctga
tcaacggcatccgggacaagcagtccggcaagacaatcctggattt
cctgaagtccgacggcttcgccaacagaaacttcatgcagctgatc
cacgacgacagcctgacctttaaagaggacatccagaaagcccagg
tgtccggccagggcgatagcctgcacgagcacattgccaatctggc
cggcagccccgccattaagaagggcatcctgcagacagtgaaggtg
gtggacgagctcgtgaaagtgatgggccggcacaagcccgagaaca
tcgtgatcgaaatggccagagagaaccagaccacccagaagggaca
gaagaacagccgcgagagaatgaagcggatcgaagagggcatcaaa
gagctgggcagccagatcctgaaagaacaccccgtggaaaacaccc
agctgcagaacgagaagctgtacctgtactacctgcagaatgggcg
ggatatgtacgtggaccaggaactggacatcaaccggctgtccgac
tacgatgtggaccatatcgtgcctcagagctttctgaaggacgact
ccatcgacaacaaggtgctgaccagaagcgacaagaaccggggcaa
gagcgacaacgtgccctccgaagaggtcgtgaagaagatgaagaac
tactggcggcagctgctgaacgccaagctgattacccagagaaagt
tcgacaatctgaccaaggccgagagaggcggcctgagcgaactgga
taaggccggcttcatcaagagacagctggtggaaacccggcagatc
acaaagcacgtggcacagatcctggactcccggatgaacactaagt
acgacgagaatgacaagctgatccgggaagtgaaagtgatcaccct
gaagtccaagctggtgtccgatttccggaaggatttccagttttac
aaagtgcgcgagatcaacaactaccaccacgcccacgacgcctacc
tgaacgccgtcgtgggaaccgccctgatcaaaaagtaccctaagct
ggaaagcgagttcgtgtacggcgactacaaggtgtacgacgtgcgg
aagatgatcgccaagagcgagcaggaaatcggcaaggctaccgcca
agtacttcttctacagcaacatcatgaactttttcaagaccgagat
taccctggccaacggcgagatccggaagcggcctctgatcgagaca
aacggcgaaaccggggagatcgtgtgggataagggccgggattttg
ccaccgtgcggaaagtgctgagcatgccccaagtgaatatcgtgaa
aaagaccgaggtgcagacaggcggcttcagcaaagagtctatcctg
cccaagaggaacagcgataagctgatcgccagaaagaaggactggg
accctaagaagtacggcggcttcgacagccccaccgtggcctattc
tgtgctggtggtggccaaagtggaaaagggcaagtccaagaaactg
aagagtgtgaaagagctgctggggatcaccatcatggaaagaagca
gcttcgagaagaatcccatcgactttctggaagccaagggctacaa
agaagtgaaaaaggacctgatcatcaagctgcctaagtactccctg
ttcgagctggaaaacggccggaagagaatgctggcctctgccggcg
aactgcagaagggaaacgaactggccctgccctccaaatatgtgaa
cttcctgtacctggccagccactatgagaagctgaagggctccccc
gaggataatgagcagaaacagctgtttgtggaacagcacaagcact
acctggacgagatcatcgagcagatcagcgagttctccaagagagt
gatcctggccgacgctaatctggacaaagtgctgtccgcctacaac
aagcaccgggataagcccatcagagagcaggccgagaatatcatcc
acctgtttaccctgaccaatctgggagcccctgccgccttcaagta
ctttgacaccaccatcgaccggaagaggtacaccagcaccaaagag
gtgctggacgccaccctgatccaccagagcatcaccggcctgtacg
agacacggatcgacctgtctcagctgggaggcgacaaaaggccggc
ggccacgaaaaaggccggccaggcaaaaaagaaaaagtaa
cas9-PCV1 Rep fusion protein gene
(SEQ ID NO: 11)
atggactataaggaccacgacggagactacaaggatcatgatattg
attacaaagacgatgacgataagatggccccaaagaagaagcggaa
ggtcggtatccacggagtcccagcagccgacaagaagtacagcatc
ggcctggacatcggcaccaactctgtgggctgggccgtgatcaccg
acgagtacaaggtgcccagcaagaaattcaaggtgctgggcaacac
cgaccggcacagcatcaagaagaacctgatcggagccctgctgttc
gacagcggcgaaacagccgaggccacccggctgaagagaaccgcca
gaagaagatacaccagacggaagaaccggatctgctatctgcaaga
gatcttcagcaacgagatggccaaggtggacgacagcttcttccac
agactggaagagtccttcctggtggaagaggataagaagcacgagc
ggcaccccatcttcggcaacatcgtggacgaggtggcctaccacga
gaagtaccccaccatctaccacctgagaaagaaactggtggacagc
accgacaaggccgacctgcggctgatctatctggccctggcccaca
tgatcaagttccggggccacttcctgatcgagggcgacctgaaccc
cgacaacagcgacgtggacaagctgttcatccagctggtgcagacc
tacaaccagctgttcgaggaaaaccccatcaacgccagcggcgtgg
acgccaaggccatcctgtctgccagactgagcaagagcagacggct
ggaaaatctgatcgcccagctgcccggcgagaagaagaatggcctg
ttcggaaacctgattgccctgagcctgggcctgacccccaacttca
agagcaacttcgacctggccgaggatgccaaactgcagctgagcaa
ggacacctacgacgacgacctggacaacctgctggcccagatcggc
gaccagtacgccgacctgtttctggccgccaagaacctgtccgacg
ccatcctgctgagcgacatcctgagagtgaacaccgagatcaccaa
ggcccccctgagcgcctctatgatcaagagatacgacgagcaccac
caggacctgaccctgctgaaagctctcgtgcggcagcagctgcctg
agaagtacaaagagattttcttcgaccagagcaagaacggctacgc
cggctacattgacggcggagccagccaggaagagttctacaagttc
atcaagcccatcctggaaaagatggacggcaccgaggaactgctcg
tgaagctgaacagagaggacctgctgcggaagcagcggaccttcga
caacggcagcatcccccaccagatccacctgggagagctgcacgcc
attctgcggcggcaggaagatttttacccattcctgaaggacaacc
gggaaaagatcgagaagatcctgaccttccgcatcccctactacgt
gggccctctggccaggggaaacagcagattcgcctggatgaccaga
aagagcgaggaaaccatcaccccctggaacttcgaggaagtggtgg
acaagggcgcttccgcccagagcttcatcgagcggatgaccaactt
cgataagaacctgcccaacgagaaggtgctgcccaagcacagcctg
ctgtacgagtacttcaccgtgtataacgagctgaccaaagtgaaat
acgtgaccgagggaatgagaaagcccgccttcctgagcggcgagca
gaaaaaggccatcgtggacctgctgttcaagaccaaccggaaagtg
accgtgaagcagctgaaagaggactacttcaagaaaatcgagtgct
tcgactccgtggaaatctccggcgtggaagatcggttcaacgcctc
cctgggcacataccacgatctgctgaaaattatcaaggacaaggac
ttcctggacaatgaggaaaacgaggacattctggaagatatcgtgc
tgaccctgacactgtttgaggacagagagatgatcgaggaacggct
gaaaacctatgcccacctgttcgacgacaaagtgatgaagcagctg
aagcggcggagatacaccggctggggcaggctgagccggaagctga
tcaacggcatccgggacaagcagtccggcaagacaatcctggattt
cctgaagtccgacggcttcgccaacagaaacttcatgcagctgatc
cacgacgacagcctgacctttaaagaggacatccagaaagcccagg
tgtccggccagggcgatagcctgcacgagcacattgccaatctggc
cggcagccccgccattaagaagggcatcctgcagacagtgaaggtg
gtggacgagctcgtgaaagtgatgggccggcacaagcccgagaaca
tcgtgatcgaaatggccagagagaaccagaccacccagaagggaca
gaagaacagccgcgagagaatgaagcggatcgaagagggcatcaaa
gagctgggcagccagatcctgaaagaacaccccgtggaaaacaccc
agctgcagaacgagaagctgtacctgtactacctgcagaatgggcg
ggatatgtacgtggaccaggaactggacatcaaccggctgtccgac
tacgatgtggaccatatcgtgcctcagagctttctgaaggacgact
ccatcgacaacaaggtgctgaccagaagcgacaagaaccggggcaa
gagcgacaacgtgccctccgaagaggtcgtgaagaagatgaagaac
tactggcggcagctgctgaacgccaagctgattacccagagaaagt
tcgacaatctgaccaaggccgagagaggcggcctgagcgaactgga
taaggccggcttcatcaagagacagctggtggaaacccggcagatc
acaaagcacgtggcacagatcctggactcccggatgaacactaagt
acgacgagaatgacaagctgatccgggaagtgaaagtgatcaccct
gaagtccaagctggtgtccgatttccggaaggatttccagttttac
aaagtgcgcgagatcaacaactaccaccacgcccacgacgcctacc
tgaacgccgtcgtgggaaccgccctgatcaaaaagtaccctaagct
ggaaagcgagttcgtgtacggcgactacaaggtgtacgacgtgcgg
aagatgatcgccaagagcgagcaggaaatcggcaaggctaccgcca
agtacttcttctacagcaacatcatgaactttttcaagaccgagat
taccctggccaacggcgagatccggaagcggcctctgatcgagaca
aacggcgaaaccggggagatcgtgtgggataagggccgggattttg
ccaccgtgcggaaagtgctgagcatgccccaagtgaatatcgtgaa
aaagaccgaggtgcagacaggcggcttcagcaaagagtctatcctg
cccaagaggaacagcgataagctgatcgccagaaagaaggactggg
accctaagaagtacggcggcttcgacagccccaccgtggcctattc
tgtgctggtggtggccaaagtggaaaagggcaagtccaagaaactg
aagagtgtgaaagagctgctggggatcaccatcatggaaagaagca
gcttcgagaagaatcccatcgactttctggaagccaagggctacaa
agaagtgaaaaaggacctgatcatcaagctgcctaagtactccctg
ttcgagctggaaaacggccggaagagaatgctggcctctgccggcg
aactgcagaagggaaacgaactggccctgccctccaaatatgtgaa
cttcctgtacctggccagccactatgagaagctgaagggctccccc
gaggataatgagcagaaacagctgtttgtggaacagcacaagcact
acctggacgagatcatcgagcagatcagcgagttctccaagagagt
gatcctggccgacgctaatctggacaaagtgctgtccgcctacaac
aagcaccgggataagcccatcagagagcaggccgagaatatcatcc
acctgtttaccctgaccaatctgggagcccctgccgccttcaagta
ctttgacaccaccatcgaccggaagaggtacaccagcaccaaagag
gtgctggacgccaccctgatccaccagagcatcaccggcctgtacg
agacacggatcgacctgtctcagctgggaggcgacaaaaggccggc
ggccacgaaaaaggccggccaggcaaaaaagaaaaaggtgagcgct
ggaggaggtggaagcggaggaggaggaagcggaggaggaggtagcc
tcgagaccatgccaagcaagaaaagcggcccgcaaccccataagag
gtgggtgttcacccttaataatccttccgaggaggagaaaaacaaa
atacgggagcttccaatctccctttttgattattttgtttgcggag
aggaaggtttggaagagggtagaactcctcacctccaggggtttgc
gaattttgctaagaagcagacttttaacaaggtgaagtggtatttt
ggtgcccgctgccacatcgagaaagcgaaaggaaccgaccagcaga
ataaagaatattgcagtaaagaaggtcacatacttatcgagtgtgg
agccccccggaaccaggggaagcgcagcgacctgtctactgctgtg
agtacccttttggagacggggtctttggtgactgtagccgagcagt
tccctgtaacgtatgtgagaaatttccgggggctggctgaactttt
gaaagtgagcgggaagatgcagcagcgtgattggaagacagctgta
cacgtcatagtgggaccgcccggttgtgggaagagccagtgggccc
gtaattttactgagcctagcgacacctactggaagcctagtagaaa
taagtggtgggatggctatcatggagaagaagttgttgttttggat
gatttttatggctggttaccttgggatgatctactgagactgtgtg
accggtatccattgactgtagagactaaaggcggtactgttccttt
tttggcccgcagtattttgattaccagcaatcaggccccccaggaa
tggtactcctcaactgctgtcccagccgtagaagctctctatcgga
ggattactactttgcaattttggaagactgctggagaacaatccac
ggaggtgcccgaaggccgatttgaagcagtggacccaccctgtgcc
cttttcccatataaaataaattactga
Pig circovirus 1 (PCV1) origin of
replication (POR)
(SEQ ID NO: 12)
ctgcgggggcgggtccttcttctgcggtaacgcctccttggccacg
tcatcctataaaagtgaaagaagtgcgctgctgtagtattaccagcg
cacttcggcagcggcagcacctcggcagcgtcagtgaaaatgccaag
caagaaaagcggcccgcaaccccataagag
triple fusion of E.coli recA gene
(SEQ ID NO: 13)
atggacgagaacaagaagcgcgccctggccgcggccctgggacagat
cgaacgccaattcggcaaaggcgcggtcatgcgcatgggcgaccatg
agcgccaggcgatcccggccatctccaccggctccctgggtctggac
atcgccctcggcatcggcggcctgcccaagggccggatcgtcgagat
ctacggtccggaatcctcgggcaagaccaccctgaccctctcggtga
tcgccgaggcccagaaacagggcgccacctgtgccttcgtcgacgcc
gagcacgcgctcgatcccgactatgccggcaagctgggcgtcaacgt
cgacgacctgctggtctcccagccggacaccggcgagcaggccctgg
aaatcaccgacatgctggtgcgctccaacgcggtcgacgtgatcatc
gtcgactccgtggccgcgctggtacccaaggccgagatcgaaggcga
gatgggcgacgcccacgtcggcctgcaggcacgcctgatgtcccagg
cgctgcgcaagatcaccggcaatatcaagaacgccaactgcctggtc
atcttcatcaaccagatccgcatgaagatcggcgtcatgttcggcaa
cccggaaaccaccaccggcggtaacgcactgaagttctacgcctcgg
tccgcctggacatccgtcgtaccggcgcggtgaaggaaggcgacgag
gtggtgggtagcgaaacccgcgtcaaggtggtgaagaacaaggtttc
cccgccgttccgccaggccgagttccagatcctctacggtaagggca
tctaccgtaccggcgagatcatcgatctgggcgtgcaattgggcctg
gtcgagaagtccggcgcctggtacagctaccagggcagcaagatcgg
ccagggcaaggcgaacgccgccaagtacctggaagacaatccggaaa
tcggttcggtactggagaagaccattcgcgaccagttgctggccaag
agcggcccggtgaaggccgacgccgaagaagtggctgacgccgaagc
cgattcagagctcggagaaggtcaaggacagggacaaggtccaggac
gaggatacgcatataagcttgacgagaacaagaagcgcgccctggcc
gcggccctgggacagatcgaacgccaattcggcaaaggcgcggtcat
gcgcatgggcgaccatgagcgccaggcgatcccggccatctccaccg
gctccctgggtctggacatcgccctcggcatcggcggcctgcccaag
ggccggatcgtcgagatctacggtccggaatcctcgggcaagaccac
cctgaccctctcggtgatcgccgaggcccagaaacagggcgccacct
gtgccttcgtcgacgccgagcacgcgctcgatcccgactatgccggc
aagctgggcgtcaacgtcgacgacctgctggtctcccagccggacac
cggcgagcaggccctggaaatcaccgacatgctggtgcgctccaacg
cggtcgacgtgatcatcgtcgactccgtggccgcgctggtacccaag
gccgagatcgaaggcgagatgggcgacgcccacgtcggcctgcaggc
acgcctgatgtcccaggcgctgcgcaagatcaccggcaatatcaaga
acgccaactgcctggtcatcttcatcaaccagatccgcatgaagatc
ggcgtcatgttcggcaacccggaaaccaccaccggcggtaacgcact
gaagttctacgcctcggtccgcctggacatccgtcgtaccggcgcgg
tgaaggaaggcgacgaggtggtgggtagcgaaacccgcgtcaaggtg
gtgaagaacaaggtttccccgccgttccgccaggccgagttccagat
cctctacggtaagggcatctaccgtaccggcgagatcatcgatctgg
gcgtgcaattgggcctggtcgagaagtccggcgcctggtacagctac
cagggcagcaagatcggccagggcaaggcgaacgccgccaagtacct
ggaagacaatccggaaatcggttcggtactggagaagaccattcgcg
accagttgctggccaagagcggcccggtgaaggccgacgccgaagaa
gtggctgacgccgaagccgattcaggatccggagaaggtcaaggaca
gggacaaggtccaggacgaggatacgcatatgcatgcgacgagaaca
agaagcgcgccctggccgcggccctgggacagatcgaacgccaattc
ggcaaaggcgcggtcatgcgcatgggcgaccatgagcgccaggcgat
cccggccatctccaccggctccctgggtctggacatcgccctcggca
tcggcggcctgcccaagggccggatcgtcgagatctacggtccggaa
tcctcgggcaagaccaccctgaccctctcggtgatcgccgaggccca
gaaacagggcgccacctgtgccttcgtcgacgccgagcacgcgctcg
atcccgactatgccggcaagctgggcgtcaacgtcgacgacctgctg
gtctcccagccggacaccggcgagcaggccctggaaatcaccgacat
gctggtgcgctccaacgcggtcgacgtgatcatcgtcgactccgtgg
ccgcgctggtacccaaggccgagatcgaaggcgagatgggcgacgcc
cacgtcggcctgcaggcacgcctgatgtcccaggcgctgcgcaagat
caccggcaatatcaagaacgccaactgcctggtcatcttcatcaacc
agatccgcatgaagatcggcgtcatgttcggcaacccggaaaccacc
accggcggtaacgcactgaagttctacgcctcggtccgcctggacat
ccgtcgtaccggcgcggtgaaggaaggcgacgaggtggtgggtagcg
aaacccgcgtcaaggtggtgaagaacaaggtttccccgccgttccgc
caggccgagttccagatcctctacggtaagggcatctaccgtaccgg
cgagatcatcgatctgggcgtgcaattgggcctggtcgagaagtccg
gcgcctggtacagctaccagggcagcaagatcggccagggcaaggcg
aacgccgccaagtacctggaagacaatccggaaatcggttcggtact
ggagaagaccattcgcgaccagttgctggccaagagcggcccggtga
aggccgacgccgaagaagtggctgacgccgaagccgattaa
triple fusion of human rad51 gene
(SEQ ID NO: 14)
atggcaatgcagatgcagcttgaagcaaatgcagatacttcagtgga
agaagaaagctttggcccacaacccatttcacggttagagcagtgtg
gcataaatgccaacgatgtgaagaaattggaagaagctggattccat
actgtggaggctgttgcctatgcgccaaagaaggagctaataaatat
taagggaattagtgaagccaaagctgataaaattctggctgaggcag
ctaaattagttccaatgggtttcaccactgcaactgaattccaccaa
aggcggtcagagatcatacagattactactggctccaaagagcttga
caaactacttcaaggtggaattgagactggatctatcacagaaatgt
ttggagaattccgaactgggaagacccagatctgtcatacgctagct
gtcacctgccagcttcccattgaccggggtggaggtgaaggaaaggc
catgtacattgacactgagggtacctttaggccagaacggctgctgg
cagtggctgagaggtatggtctctctggcagtgatgtcctggataat
gtagcatatgctcgagcgttcaacacagaccaccagacccagctcct
ttatcaagcatcagccatgatggtagaatctaggtatgcactgctta
ttgtagacagtgccaccgccctttacagaacagactactcgggtcga
ggtgagctttcagccaggcagatgcacttggccaggtttctgcggat
gcttctgcgactcgctgatgagtttggtgtagcagtggtaatcacta
atcaggtggtagctcaagtggatggagcagcgatgtttgctgctgat
cccaaaaaacctattggaggaaatatcatcgcccatgcatcaacaac
cagattgtatctgaggaaaggaagaggggaaaccagaatctgcaaaa
tctacgactctccctgtcttcctgaagctgaagctatgttcgccatt
aatgcagatggagtgggagatgccaaagacgagctcggagaaggtca
aggacagggacaaggtccaggacgaggatacgcatataagcttatgg
caatgcagatgcagcttgaagcaaatgcagatacttcagtggaagaa
gaaagctttggcccacaacccatttcacggttagagcagtgtggcat
aaatgccaacgatgtgaagaaattggaagaagctggattccatactg
tggaggctgttgcctatgcgccaaagaaggagctaataaatattaag
ggaattagtgaagccaaagctgataaaattctggctgaggcagctaa
attagttccaatgggtttcaccactgcaactgaattccaccaaaggc
ggtcagagatcatacagattactactggctccaaagagcttgacaaa
ctacttcaaggtggaattgagactggatctatcacagaaatgtttgg
agaattccgaactgggaagacccagatctgtcatacgctagctgtca
cctgccagcttcccattgaccggggtggaggtgaaggaaaggccatg
tacattgacactgagggtacctttaggccagaacggctgctggcagt
ggctgagaggtatggtctctctggcagtgatgtcctggataatgtag
catatgctcgagcgttcaacacagaccaccagacccagctcctttat
caagcatcagccatgatggtagaatctaggtatgcactgcttattgt
agacagtgccaccgccctttacagaacagactactcgggtcgaggtg
agctttcagccaggcagatgcacttggccaggtttctgcggatgctt
ctgcgactcgctgatgagtttggtgtagcagtggtaatcactaatca
ggtggtagctcaagtggatggagcagcgatgtttgctgctgatccca
aaaaacctattggaggaaatatcatcgcccatgcatcaacaaccaga
ttgtatctgaggaaaggaagaggggaaaccagaatctgcaaaatcta
cgactctccctgtcttcctgaagctgaagctatgttcgccattaatg
cagatggagtgggagatgccaaagacggatccggagaaggtcaagga
cagggacaaggtccaggacgaggatacgcatatgcatgcatggcaat
gcagatgcagcttgaagcaaatgcagatacttcagtggaagaagaaa
gctttggcccacaacccatttcacggttagagcagtgtggcataaat
gccaacgatgtgaagaaattggaagaagctggattccatactgtgga
ggctgttgcctatgcgccaaagaaggagctaataaatattaagggaa
ttagtgaagccaaagctgataaaattctggctgaggcagctaaatta
gttccaatgggtttcaccactgcaactgaattccaccaaaggcggtc
agagatcatacagattactactggctccaaagagcttgacaaactac
ttcaaggtggaattgagactggatctatcacagaaatgtttggagaa
ttccgaactgggaagacccagatctgtcatacgctagctgtcacctg
ccagcttcccattgaccggggtggaggtgaaggaaaggccatgtaca
ttgacactgagggtacctttaggccagaacggctgctggcagtggct
gagaggtatggtctctctggcagtgatgtcctggataatgtagcata
tgctcgagcgttcaacacagaccaccagacccagctcctttatcaag
catcagccatgatggtagaatctaggtatgcactgcttattgtagac
agtgccaccgccctttacagaacagactactcgggtcgaggtgagct
ttcagccaggcagatgcacttggccaggtttctgcggatgcttctgc
gactcgctgatgagtttggtgtagcagtggtaatcactaatcaggtg
gtagctcaagtggatggagcagcgatgtttgctgctgatcccaaaaa
acctattggaggaaatatcatcgcccatgcatcaacaaccagattgt
atctgaggaaaggaagaggggaaaccagaatctgcaaaatctacgac
tctccctgtcttcctgaagctgaagctatgttcgccattaatgcaga
tggagtgggagatgccaaagactga
MSH2 dominant-negative gene sequence (M688R)
(SEQ ID NO: 15)
atggcggtgcagccgaaggagacgctgcagttggagagcgcggccga
ggtcggcttcgtgcgcttctttcagggcatgccggagaagccgacca
ccacagtgcgccttttcgaccggggcgacttctatacggcgcacggc
gaggacgcgctgctggccgcccgggaggtgttcaagacccagggggt
gatcaagtacatggggccggcaggagcaaagaatctgcagagtgttg
tgcttagtaaaatgaattttgaatcttttgtaaaagatcttcttctg
gttcgtcagtatagagttgaagtttataagaatagagctggaaataa
ggcatccaaggagaatgattggtatttggcatataaggcttctcctg
gcaatctctctcagtttgaagacattctctttggtaacaatgatatg
tcagcttccattggtgttgtgggtgttaaaatgtccgcagttgatgg
ccagagacaggttggagttgggtatgtggattccatacagaggaaac
taggactgtgtgaattccctgataatgatcagttctccaatcttgag
gctctcctcatccagattggaccaaaggaatgtgttttacccggagg
agagactgctggagacatggggaaactgagacagataattcaaagag
gaggaattctgatcacagaaagaaaaaaagctgacttttccacaaaa
gacatttatcaggacctcaaccggttgttgaaaggcaaaaagggaga
gcagatgaatagtgctgtattgccagaaatggagaatcaggttgcag
tttcatcactgtctgcggtaatcaagtttttagaactcttatcagat
gattccaactttggacagtttgaactgactacttttgacttcagcca
gtatatgaaattggatattgcagcagtcagagcccttaacctttttc
agggttctgttgaagataccactggctctcagtctctggctgccttg
ctgaataagtgtaaaacccctcaaggacaaagacttgttaaccagtg
gattaagcagcctctcatggataagaacagaatagaggagagattga
atttagtggaagcttttgtagaagatgcagaattgaggcagacttta
caagaagatttacttcgtcgattcccagatcttaaccgacttgccaa
gaagtttcaaagacaagcagcaaacttacaagattgttaccgactct
atcagggtataaatcaactacctaatgttatacaggctctggaaaaa
catgaaggaaaacaccagaaattattgttggcagtttttgtgactcc
tcttactgatcttcgttctgacttctccaagtttcaggaaatgatag
aaacaactttagatatggatcaggtggaaaaccatgaattccttgta
aaaccttcatttgatcctaatctcagtgaattaagagaaataatgaa
tgacttggaaaagaagatgcagtcaacattaataagtgcagccagag
atcttggcttggaccctggcaaacagattaaactggattccagtgca
cagtttggatattactttcgtgtaacctgtaaggaagaaaaagtcct
tcgtaacaataaaaactttagtactgtagatatccagaagaatggtg
ttaaatttaccaacagcaaattgacttctttaaatgaagagtatacc
aaaaataaaacagaatatgaagaagcccaggatgccattgttaaaga
aattgtcaatatttcttcaggctatgtagaaccaatgcagacactca
atgatgtgttagctcagctagatgctgttgtcagctttgctcacgtg
tcaaatggagcacctgttccatatgtacgaccagccattttggagaa
aggacaaggaagaattatattaaaagcatccaggcatgcttgtgttg
aagttcaagatgaaattgcatttattcctaatgacgtatactttgaa
aaagataaacagatgttccacatcattactggccccaatatgggagg
taaatcaacatatattcgacaaactggggtgatagtactccgggccc
aaattgggtgttttgtgccatgtgagtcagcagaagtgtccattgtg
gactgcatcttagcccgagtaggggctggtgacagtcaattgaaagg
agtctccacgttcatggctgaaatgttggaaactgcttctatcctca
ggtctgcaaccaaagattcattaataatcatagatgaattgggaaga
ggaacttctacctacgatggatttgggttagcatgggctatatcaga
atacattgcaacaaagattggtgctttttgcatgtttgcaacccatt
ttcatgaacttactgccttggccaatcagataccaactgttaataat
ctacatgtcacagcactcaccactgaagagaccttaactatgcttta
tcaggtgaagaaaggtgtctgtgatcaaagttttgggattcatgttg
cagagcttgctaatttccctaagcatgtaatagagtgtgctaaacag
aaagccctggaacttgaggagtttcagtatattggagaatcgcaagg
atatgatatcatggaaccagcagcaaagaagtgctatctggaaagag
agaacctcagagtgacagagccaaaagaccagtgcctcattttgctg
acatggaaaaggaaacttcgtgggggaaagagatctgcttgcagtcg
gccagagagacagaaccagggcagtgcgactccaagtgcatcagcat
ag
MHS6 dominant-negative gene sequence from
Saccharomyces cerevisiae
(SEQ ID NO: 16)
atggccccagctacccctaaaacttctaagactgcacacttcgaaaa
tggctccacatcttctcaaaagaaaatgaagcaatcgagtttgttat
cttttttctcaaaacaggtaccttctggcacaccgtcaaagaaggtc
cagaagcctactccagcgactttagaaaatacagctactgataagat
aacaaagaatccacaaggaggaaagacaggcaaacttttcgtagatg
tagacgaagacaatgatttgacaatagctgaagaaacggtatcaact
gtaaggagtgacataatgcattctcaagagccccaatctgatactat
gttaaatagtaacacgacagaacctaaaagtaccactacggacgagg
atttatcatcttcccaatctcgaaggaatcacaaaaggagagtgaat
tatgccgaaagtgatgatgatgactcagatactacattcactgctaa
acggaagaagggtaaggtagttgatagtgaaagcgatgaggatgaat
atttaccagataaaaatgacggcgatgaagatgacgatattgcagac
gacaaagaggacatcaaaggagaattggcagaagatagtggtgatga
cgatgatttaatctctctggcagaaacgacttctaagaaaaaatttt
catacaacacttcgcattcttcatcgccctttacaagaaatatatcc
cgtgacaactcaaaaaagaaaagcaggccaaatcaggcaccaagtag
atcgtacaatccttctcatagtcaaccatcagcgacttctaagtcta
gcaaattcaataaacaaaatgaagaacgatatcaatggttagtggat
gaacgagatgctcagcgccgtcccaagagtgatccagagtacgatcc
aagaacactgtacatcccatcttctgcatggaacaagtttactccgt
ttgaaaaacaatattgggaaattaaatccaaaatgtgggattgtatt
gtctttttcaaaaagggtaaggcctttgaattatatgaaaaggatgc
attattggctaatgcattatttgacttaaagattgcaggtggaggac
gcgctaatatgcaactagctgggattccagagatgtcatttgaatat
tgggccgctcagtttatccaaatgggatataaagttgcgaaagtgga
tcaaagagagtcaatgttggctaaagaaatgagggaaggttctaaag
gtatcgttaaaagagaactccagtgcatattgacatcgggtacgtta
actgatggagatatgttgcattcggatctggctactttttgtcttgc
catcagggaagaacctggtaacttctacaatgaaactcagctagatt
catcaacgatagtgcaaaagctgaatacaaaaatattcggtgcagca
ttcattgatactgcaactggtgagcttcaaatgctggagtttgaaga
tgatagtgaatgcaccaaattggacacattaatgtcgcaagtaagac
ctatggaagtcgtcatggagaggaataacttaagcacactagctaac
aaaattgtaaaattcaattcagcacccaatgcaatttttaatgaggt
taaagccggtgaagaattttatgactgcgataaaacctatgctgaaa
tcatatcatcagaatacttttctacggaagaagattggccagaggtt
ttgaaatcctattatgatacgggtaaaaaagttggctttagtgcatt
tggtggattattgtattatttgaagtggttaaaactggataagaatt
taatttcgatgaaaaatatcaaagagtacgattttgtaaaatcgcaa
cactccatggttttagatggtatcactttacagaacttggaaatatt
ttccaattcgtttgatggttctgataagggtactttgttcaaattat
tcaacagggctattactccaatgggtaagagaatgatgaaaaagtgg
ttaatgcatccattattacgtaagaatgatattgaaagcaggctcga
tagtgttgactctttgttacaagatattacattaagagaacagctag
agataacattttcaaaattgccagatttggaaagaatgttggcccgt
atccatagtaggacaattaaagtgaaagattttgaaaaggtaattac
cgcttttgaaacgattattgaattacaagactctttaaagaacaacg
atttgaagggagatgtttcaaaatacatatcctctttccctgaggga
cttgtggaagctgtcaaaagctggactaatgcttttgaaagacaaaa
agcaatcaatgaaaatatcattgtaccacaaaggggatttgacatag
agtttgataagtcaatggatagaatacaggagctagaagacgaattg
atggaaattctgatgacttataggaagcaattcaaatgttccaatat
acaatataaagattccggaaaggagatatacactattgaaattccta
tctctgcaactaaaaatgtcccatcaaattgggttcaaatggctgcg
aacaaaacgtacaagagatactactctgatgaagtaagagctttagc
aagatcgatggcggaggctaaagaaatacacaagacgttagaggagg
acctaaaaaatagattatgtcaaaaatttgatgcgcattataataca
atttggatgccaaccatacaggccatctctaacatagattgtttgtt
ggcaattacaaggacatctgaatatctaggtgctccctcttgtaggc
caaccattgtagatgaggtggactcgaaaacgaatactcaattaaat
ggttttctgaaatttaaatcattaagacatccatgctttaacctcgg
cgctactacagcaaaggacttcattcctaacgacattgaattaggta
aagaacagcccagattaggattgttaacgggtgccaacgcagctggt
aaatccacgatattgaggatggcatgtattgccgtaattatggcaca
aatgggatgttatgttccttgtgaatctgccgttctgacgccgattg
atagaatcatgactcgtttaggcgctaatgataatatcatgcaaggt
aaatctactttttttgtggaattggcggaaacaaaaaaaatattaga
catggctaccaataggtcattattagttgttgacgagttgggaagag
gaggttcatcgagtgatggttttgcaattgcagaaagtgtgctacat
catgtcgcgacacatattcaaagtctgggtttttttgccacacatta
tgggacattggcatcaagtttcaagcaccatcctcaagtaagaccac
tgaaaatgagcattctagtcgatgaagcgactaggaacgttacattt
ttgtataagatgcttgaaggacaaagtgaaggctcttttggtatgca
tgttgcgtcaatgtgtggcatttcaaaggaaattattgataacgcgc
agattgccgctgataatttagaacatacttctaggctagtcaaagaa
cgtgatttagctgcaaataatttgaatggtgaagttgtctctgtgcc
aggtggcttacaaagtgatttcgttcgaattgcgtacggagatggac
tgaagaatacaaaattaggttctggtgaaggggtcttgaactatgat
tggaacataaaaaggaacgttttgaaaagtctatttagtataattga
tgatttacaatcctaa
Bacteriophage T4 endonuclease VII
(SEQ ID NO: 17)
atgttattgactggcaaattatacaaagaagaaaaacagaaatttta
tgatgcacaaaacggtaaatgcttaatttgccaacgagaactaaatc
ctgatgttcaagctaatcacctcgaccatgaccatgaattaaatgga
ccaaaagcaggaaaggtgcgtggattgctttgtaatctatgcaatgc
tgcagaaggtcaaatgaagcataaatttaatcgttctggcttaaagg
gacaaggtgttgattatcttgaatggttagaaaatttacttacttat
ttaaaatccgattacacccaaaataatattcaccctaactttgttgg
agataaatcaaaggaattttctcgtttaggaaaagaggaaatgatgg
ccgagatgcttcaaagaggatttgaatataatgaatctgacaccaaa
acacaattaatagcttcattcaagaagcagcttagaaagagtttaaa
atga
CCE1 cruciform cutting endonuclease from
Saccharomyces cerevisiae
(SEQ ID NO: 18)
ATGTCGACAGCACAGAAAGCTAAGATATTGCAACTCATCGATTCCT
GCTGCCAAAATGCAAAAAGCACACAACTGAAATCTTTATCATTTGT
TATTGGAGCAGTAAATGGCACGACGAAAGAAGCTAAAAGAACCTAC
ATTCAAGAACAGTGTGAATTTTTGGAGAAGTTACGACAACAAAAGA
TAAGAGAGGGAAGAATTAACATATTGTCTATGGATGCTGGTGTTTC
TAACTTTGCTTTCTCTAAGATGCAATTGCTCAATAATGATCCGCTC
CCTAAAGTACTAGACTGGCAAAAGATAAATCTAGAGGAGAAATTTT
TTCAAAACCTCAAAAAGTTAAGCTTGAATCCTGCTGAAACTTCTGA
GCTTGTATTTAACCTTACGGAGTATTTATTTGAATCTATGCCGATA
CCAGATATGTTTACAATTGAAAGGCAACGTACCAGAACTATGTCTT
CGAGGCATATTTTAGACCCAATTTTAAAAGTGAATATTCTCGAACA
GATTCTTTTCTCTAACTTGGAAAATAAAATGAAGTATACGAATAAA
ATACCGAATACGTCCAAGTTGAGGTATATGGTATGTTCGTCCGATC
CACATCGGATGACTTCATATTGGTGCATTCCAAGAGAAGAGACACC
GACCAGTTCAAAAAAGTTAAAATCTAACAAACATAGCAAAGATTCT
CGAATAAAGCTAGTGAAAAAAATACTTTCAACCTCAATACTAGAAG
GTAATTCAACTAGTTCTACAAAACTGGTCGAGTTCATAGGAGTTTG
GAATAATAGGATAAGAAATGCCCTTACCAAAAAAAAAAGTTTCAAG
CTATGTGATATACTAGAGATCCAAGATAATTCGGGGGTGAGAAAAG
ATGACGATTTGGCAGATTCATTCCTCCATTGTTTGTCTTGGATGGA
GTGGTTAAAAAATTATGAAAGTATTACTGAACTCTTGAATTCAAAA
ACACTGGTTAAAACACAGTTCGGACAGGTGTTTGAATTTTGTGAAA
ATAAGGTACAAAAGCTGAAATTTTTGCAGAACACTTACAACAATGA
CTAA
Human GEN1 Holliday junction 5′ flap
endonuclease
(SEQ ID NO: 19)
atgggagtgaatgacttgtggcaaattttggagcctgttaagcaa
cacatccccttgcgtaatcttggtgggaaaaccattgcagttgat
ctgagtctctgggtgtgtgaggcacagacagtcaaaaaaatgatg
ggcagcgtcatgaagccccacctcaggaacttattttttcgtatc
tcatatttaacacaaatggatgtaaaactggtatttgttatggaa
ggggaaccaccaaagctgaaagctgatgtcataagcaagaggaat
cagtctcggtatgggtcttctggaaaatcgtggtctcagaaaaca
gggagatcacattttaaatcagtcttaagagagtgcctccatatg
ctcgaatgcttaggaatcccctgggttcaggctgctggggaagct
gaagccatgtgtgcttatctcaatgctggtggtcatgtcgatggc
tgcctcaccaatgatggagatactttcctttatggggcccagact
gtttacaggaatttcactatgaatacaaaggacccacatgttgac
tgttacacaatgtcatctatcaagagtaaactaggtttggataga
gatgctctggttggattagcaatacttcttggctgtgattatctc
ccaaagggagtccctggagttggaaaagagcaagcattaaaactt
atacagattttgaaagggcaaagtttacttcagaggtttaatcgg
tggaatgaaacatcttgtaactctagtccacaactgctagtcact
aaaaaactggctcattgttccgtatgttcccatccaggttcacct
aaggatcatgaacgtaatggatgcagattatgtaaaagtgataaa
tattgtgagccacatgactatgaatactgctgtccttgtgagtgg
caccgtacagaacatgataggcaactcagtgaagtagagaacaat
attaagaagaaagcttgctgttgtgagggattcccattccatgag
gttattcaagaattccttttaaacaaggataaattggtgaaggtt
atcaggtaccaaagacctgatttgttattgtttcagagatttact
cttgaaaaaatggagtggcccaatcactatgcatgtgagaaattg
ctggtacttttgacccattatgacatgatagaaagaaagcttggt
agcagaaactctaatcaactacagccaattcgaattgttaagact
cgaatcagaaatggagttcattgttttgaaatagaatgggaaaag
cctgaacattatgctatggaagataaacaacatggagaatttgct
ttattaacaattgaggaagaatcattgtttgaagcagcatatcct
gagatcgttgctgtttaccaaaaacaaaagttagaaattaaaggg
aagaaacaaaaacgtattaagcctaaagaaaacaatttgccagaa
ccagatgaagtaatgagctttcagtcacacatgactttaaaaccc
acatgtgaaatctttcataagcagaattccaagttaaattcgggg
atttcccctgatcctacattaccacaggaatctatttctgcctca
ttgaatagcttgcttttacctaaaaatactccatgtttgaatgca
caagaacagttcatgtcttctctaagacctttggctatacagcaa
attaaagctgtcagtaagtctctaatttcagaatctagtcaaccc
aatacctcatctcataatatatccgtgattgctgatctacacttg
agcactattgactgggaaggtacttcttttagtaattctccagct
attcaaaggaatactttttctcatgatttaaaatcagaagttgaa
tcagagctatcagccatccctgatggctttgaaaatatcccagaa
caactgtcctgtgaatcagaaaggtacactgcaaacataaagaaa
gtgttggatgaggattctgatgggattagtcctgaagagcatcta
ctttctggcattactgatttatgtcttcaggatttgcctttaaag
gaacgaatatttacaaaattatcatatcctcaggataatctacaa
ccagatgtcaacctgaaaactttgtccatacttagtgtaaaagaa
tcttgtattgctaacagtggttctgattgtacatcacatctttca
aaggatcttccaggaattcccttgcaaaatgaatccagagactct
aaaattctaaaaggagaccagctgcttcaagaagactataaagtc
aatacttctgtcccttattctgtcagtaacacagtggtaaagacc
tgcaatgttagaccaccaaatactgctttagatcatagtagaaaa
gttgatatgcaaaccactcggaaaattttaatgaagaagagtgtt
tgccttgacagacattcctctgatgaacaaagtgccccagtgttt
gggaaagctaagtacacaactcaaagaatgaagcacagttctcaa
aagcataattcatcccatttcaaagaaagtggccataacaagttg
agtagccctaagatacatattaaagaaactgaacagtgtgtcaga
tcttatgaaacagctgaaaatgaagaaagctgtttcccagattca
acaaaaagttctctgagttctctacaatgtcataagaaagaaaac
aactctggtacttgtttggatagccctcttcctttacgccagaga
ttaaaactaagattccaaagcacttga
Arabidopsis AtGENI resolvase,
(SEQ ID NO: 20)
atgggtgtgggaggcaatttctgggatttgctgagaccatatgct
cagcaacaaggctttgattttctcagaaacaaacgagtcgctgtt
gatctctccttctggatcgttcagcatgaaaccgctgttaagggt
ttcgtccttaaacctcacctccgactcactttcttccgtactatc
aacctcttctcaaagtttggagcgtacccggtttttgtggttgat
ggaacaccatcacctttgaaatctcaggcgagaatctccaggttt
ttccgttcttctggaattgatacttgtaatctacctgtgattaaa
gatggtgtctcggttgagagaaacaagctgttttctgaatgggtt
agggaatgtgtggagctactcgaattgctcggtattccggtgctg
aaagctaatggtgaggctgaagctctctgtgcacagttaaacagc
caaggttttgtggatgcttgcattactcctgatagtgatgctttc
ctttttggtgctatgtgcgtgatcaaagacatcaagcctaattca
agagaaccttttgaatgctaccatatgtcacatatcgagtctggc
ctcggtttgaagcggaaacacttgattgctatttctctattggtg
ggaaacgattatgattcaggcggtgttcttgggattggtgtggat
aaagcactgcgcattgttcgtgagttttctgaagaccaagtactt
gaaagactacaggacattggaaatgggttgcaacctgcagttcct
ggtggaatcaaatccggggatgatggtgaagaattccgctcagag
atgaaaaaaagatctcctcactgttcccgttgtggacacctgggc
agcaagagaactcattttaagtcctcttgtgagcactgcggttgt
gatagtggttgcattaaaaaaccattagggtttagatgtgaatgc
tccttttgttccaaggatcgagatttaagggaacaaaagaaaacc
aatgattggtggatcaaagtctgcgataagattgctctagcgcca
gagtttcccaacagaaagattattgaactttatctatccgatggt
ttgatgacaggagatggatcgtcaatgtcttggggaactcctgat
actggaatgctagtggatctcatggttttcaaactgcactgggac
ccatcttatgttagaaaaatgttgcttccgatgctgtcgaccatt
tatctgagagaaaaggcaagaaacaacacaggatacgctttgttg
tgtgatcaatacgaatttcattcaatcaagtgcataaaaactaga
tatgggcatcagtcctttgtaataaggtggagaaaacccaaatct
acaagtggttatagtcatagtcacagcgagccagaagaatcaatt
gttgtattggaagaagaagaagagtctgttgatccgttggatggt
ttaaatgaacctcaggtgcaaaatgataatggtgactgcttcttg
ctaactgatgaatgcataggacttgttcagtctgctttccctgat
gaaacagagcattttctacatgagaagaaactgagagagtcgaaa
aagaagaatgtttctgaagaagaaacagcaacaccaagagcaaca
acaatgggtgtacaaagaagcattaccgatttctaccgttcagcg
aagaaagcagcagcaggtcaaagtatagagacaggcgggagttca
aaagcttctgcggaaaagaagagacaggcaacttctactagtagt
agtaaccttacaaagtcggtcaggcgtcgtctcttgtttggatag
Neomycin phosphotransferase selection gene
(SEQ ID NO: 21)
atgattgaacaagatggattgcacgcaggttctccggccgcttgg
gtggagaggctattcggctatgactgggcacaacagacaatcggc
tgctctgatgccgccgtgttccggctgtcagcgcaggggcgcccg
gttctttttgtcaagaccgacctgtccggtgccctgaatgaactg
caggacgaggcagcgcggctatcgtggctggccacgacgggcgtt
ccttgcgcagctgtgctcgacgttgtcactgaagcgggaagggac
tggctgctattgggcgaagtgccggggcaggatctcctgtcatct
caccttgctcctgccgagaaagtatccatcatggctgatgcaatg
cggcggctgcatacgcttgatccggctacctgcccattcgaccac
caagcgaaacatcgcatcgagcgagcacgtactcggatggaagcc
ggtcttgtcgatcaggatgatctggacgaagagcatcaggggctc
gcgccagccgaactgttcgccaggctcaaggcgcgcatgcccgac
ggcgaggatctcgtcgtgacccatggcgatgcctgcttgccgaat
atcatggtggaaaatggccgcttttctggattcatcgactgtggc
cggctgggtgtggcggaccgctatcaggacatagcgttggctacc
cgtgatattgctgaagagcttggcggcgaatgggctgaccgcttc
ctcgtgctttacggtatcgccgctcccgattcgcagcgcatcgcc
ttctatcgccttcttgacgagttcttctga
Puromycin-n-acetyltransferas selection gene
(SEQ ID NO: 22)
atgaccgagtacaagcccacggtgcgcctcgccacccgcgacgac
gtcccccgggccgtacgcaccctcgccgccgcgttcgccgactac
cccgccacgcgccacaccgtcgacccggaccgccacatcgagcgg
gtcaccgagctgcaagaactcttcctcacgcgcgtcgggctcgac
atcggcaaggtgtgggtcgcggacgacggcgccgcggtggcggtc
tggaccacgccggagagcgtcgaagcgggggcggtgttcgccgag
atcggcccgcgcatggccgagttgagcggttcccggctggccgcg
cagcaacagatggaaggcctcctggcgccgcaccggcccaaggag
cccgcgtggttcctggccaccgtcggcgtgtcgcccgaccaccag
ggcaagggtctgggcagcgccgtcgtgctccccggagtggaggcg
gccgagcgcgccggggtgcccgccttcctggagacctccgcgccc
cgcaacctccccttctacgagcggctcggcttcaccgtcaccgcc
gacgtcgaggtgcccgaaggaccgcgcacctggtgcatgacccgc
aagcccggtgcctga
SV40 poly A
(SEQ ID NO: 23)
cagacatgataagatacattgatgagtttggacaaaccacaacta
gaatgcagtgaaaaaaatgctttatttgtgaaatttgtgatgcta
ttgctttatttgtaaccattataagctgcaataaacaagttaaca
acaacaattgcattcattttatgtttcaggttcagggggaggtgt
gggaggttttttaaagcaagtaaaacctctacaaatgtggta
Origin of replication of torque teno virus
(SEQ ID NO: 24)
cggtgaagccacggagggagatctccgcgtcccgagggcgggtg
ccgaaggtgagtttacacaccgaagtcaaggggcaattcgggct
cgggactggccgggctatgggcaaggctctgaaaaaagcatgtt
tattggcaggcattacagaaagaaaagggcgctgtcactgtgtg
ctgtgcgaacaacaaagaaggcttgcaaactactaatagtaatg
tggaccccacctcgcaatgatcaacactacctta
MS2-λ-exo fusion protein
(SEQ ID NO: 25)
atggcttcaaactttactcagttcgtgctcgtggacaatggtgg
gacaggggatgtgacagtggctccttctaatttcgctaatgggg
tggcagagtggatcagctccaactcacggagccaggcctacaag
gtgacatgcagcgtcaggcagtctagtgcccagaagagaaagta
taccatcaaggtggaggtccccaaagtggctacccagacagtgg
gcggagtcgaactgcctgtcgccgcttggagatcctacctgaac
atggaactcactatcccaattttcgctaccaattctgactgtga
actcatcgtgaaggcaatgcaggggctcctcaaagacggcaatc
ctatcccttccgccatcgccgctaactcaggcatctacagcgct
ggaggaggtggaagcggaggaggaggaagcggaggaggaggtag
cggacctaagaaaaagaggaaggtggctgccgctggatccatga
caccggacattatcctgcagcgtaccgggatcgatgtgagagct
gtcgaacagggggatgatgcgtggcacaaattacggctcggcgt
catcaccgcttcagaagttcacaacgtgatagcaaaaccccgct
ccggaaagaagtggcctgacatgaaaatgtcctacttccacacc
ctgcttgctgaggtttgcaccggtgtggctccggaagttaacgc
taaagcactggcctggggaaaacagtacgagaacgacgccagaa
ccctgtttgaattcacttccggcgtgaatgttactgaatccccg
atcatctatcgcgacgaaagtatgcgtaccgcctgctctcccga
tggtttatgcagtgacggcaacggccttgaactgaaatgcccgt
ttacctcccgggatttcatgaagttccggctcggtggtttcgag
gccataaagtcagcttacatggcccaggtgcagtacagcatgtg
ggtgacgcgaaaaaatgcctggtactttgccaactatgacccgc
gtatgaagcgtgaaggcctgcattatgtcgtgattgagcgggat
gaaaagtacatggcgagttttgacgagatcgtgccggagttcat
cgaaaaaatggacgaggcactggctgaaattggttttgtatttg
gggagcaatggcgatga
topoisomerase I donor
(SEQ ID NO: 26)
gtccggcagagagctgtagccctgtacttcatcgacaagcttgc
tctgagagcaggcaatgaaaaggaggaaggagaaacagcggaca
ctgtgggctgctgctcacttcgtgtggagcacatcaatctacac
ccagagttggatggtcaggaatatgtggtagagtttgacttcct
cgggaaggactccatcagatactataacaaggtccctgttgaga
aacgagtttttaagaacctacaactatttatggagaacaagcag
cccgaggatgatctttttgatagactcaatactggtattctgaa
taagcatcttcaggatctcatggagggcttgacagccaaggtat
tccgtacgtacaatgcctccatcacgctacagcagcagctaaaa
gaactgacagccccggatgagaacatcccagcgaagatcctttc
ttataaccgtgccaatcgagctgttgcaattctttgtaaccatc
agagggcaccaccaaaaacttttgagaagtctatgatgaacttg
caaactaagattgatgccaagaaggaacagctagcagatgcccg
gagagacctgaaaagtgctaaggctgatgccaaggtcatgaagg
atgcaaagacgaagaaggtagtagagtcaaagaagaaggctgtt
cagagactggaggaacagttgatgaagctggaagttcaagccac
agaccgagaggaaaataaacagattgccctgggaacctccaaac
tcagctatctggaccctaggatcacagtggcttggtgcaagaag
tggggtgtcccaattgagaagatttacaacaaaacccagcggga
gaagtttgcctgggccattgacatggctgatgaagactatgagt
tttagccagtctcaagaggcagagttctgtgaagaggaacagtg
tggtttgggaaagatggataaactgagcctcacttgccctcgtg
cctgggggagagaggcagcaagtcttaacaaaccaacatctttg
cgaaaagataaacctggagatattataagggagagctgagccag
ttgtcctatggacaacttatttaaaaatatttcagatatcaaaa
ttctagctgtatgatttgttttgaattttgtttttattttcaag
agggcaagtggatgggaatttgtcagcgttctaccaggcaaatt
cactgtttcactgaaatgtttggattctcttagctactgtatgc
aaagtccgattatattggtgcgtttttacagttagggttttgca
ataacttctatattttaatagaaataaattcctaaactcccttc
cctctctcccatttcaggaatttaaaattaagtagaacaaaaaa
cccagcgcacctgttagagtcgtcactctctattgtcatgggga
tcaattttcattaaacttgaagcagtcgtggctttggcagtgtt
ttggttcagacacctgttcacagaaaaagcatgatgggaaaata
tttcc
Topoisomerase I wtih eGFP insertion donor
gene
(SEQ ID NO: 27)
cagcggacactgtgggctgctgctcacttcgtgtggagcacatc
aatctacacccagagttggatggtcaggaatatgtggtagagtt
tgacttcctcgggaaggactccatcagatactataacaaggtcc
ctgttgagaaacgagtttttaagaacctacaactatttatggag
aacaagcagcccgaggatgatctttttgatagactcaatactgg
tattctgaataagcatcttcaggatctcatggagggcttgacag
ccaaggtattccgtacgtacaatgcctccatcacgctacagcag
cagctaaaagaactgacagccccggatgagaacatcccagcgaa
gatcctttcttataaccgtgccaatcgagctgttgcaattcttt
gtaaccatcagagggcaccaccaaaaacttttgagaagtctatg
atgaacttgcaaactaagattgatgccaagaaggaacagctagc
agatgcccggagagacctgaaaagtgctaaggctgatgccaagg
tcatgaaggatgcaaagacgaagaaggtagtagagtcaaagaag
aaggctgttcagagactggaggaacagttgatgaagctggaagt
tcaagccacagaccgagaggaaaataaacagattgccctgggaa
cctccaaactcagctatctggaccctaggatcacagtggcttgg
tgcaagaagtggggtgtcccaattgagaagatttacaacaaaac
ccagcgggagaagtttgcctgggccattgacatggctgatgaag
actatgagtttaagggcgaggagctgttcaccggggtggtgccc
atcctggtcgagctggacggcgacgtaaacggccacaagttcag
cgtgtccggcgagggcgagggcgatgccacctacggcaagctga
ccctgaagttcatctgcaccaccggcaagctgcccgtgccctgg
cccaccctcgtgaccaccctgacctacggcgtgcagtgcttcag
ccgctaccccgaccacatgaagcagcacgacttcttcaagtccg
ccatgcccgaaggctacgtccaggagcgcaccatcttcttcaag
gacgacggcaactacaagacccgcgccgaggtgaagttcgaggg
cgacaccctggtgaaccgcatcgagctgaagggcatcgacttca
aggaggacggcaacatcctggggcacaagctggagtacaactac
aacagccacaacgtctatatcatggccgacaagcagaagaacgg
catcaaggtgaacttcaagatccgccacaacatcgaggacggca
gcgtgcagctcgccgaccactaccagcagaacacccccatcggc
gacggccccgtgctgctgcccgacaaccactacctgagcaccca
gtccgccctgagcaaagaccccaacgagaagcgcgatcacatgg
tcctgctggagttcgtgaccgccgccgggatcactctcggcatg
gacgagctatacaagccagtctcaagaggcagagttctgtgaag
aggaacagtgtggtttgggaaagatggataaactgagcctcact
tgccctcgtgcctgggggagagaggcagcaagtcttaacaaacc
aacatctttgcgaaaagataaacctggagatattataagggaga
gctgagccagttgtcctatggacaacttatttaaaaatatttca
gatatcaaaattctagctgtatgatttgttttgaattttgtttt
tattttcaagagggcaagtggatgggaatttgtcagcgttctac
caggcaaattcactgtttcactgaaatgtttggattctcttagc
tactgtatgcaaagtccgattatattggtgcgtttttacagtta
gggttttgcaataacttctatattttaatagaaataaattccta
aactcccttccctctctcccatttcaggaatttaaaattaagta
gaacaaaaaacccagcgcacctgttagagtcgtcactctctatt
gtcatggggatcaattttcattaaacttgaagcagtcgtggctt
tggcagtgttttggttcagacacctgttcacagaaaaagcatga
tgggaaaatatttcctgacttgagtgttcctttttaaatgtgaa
ttttttttttttttaattattttaaaatatttaaacctttttct
tgatcttaaagatcgtgtagattggggttggggagggatgaagg
gcgagtgaatctaaggataatgaaataatcagtgactgaaacca
ttttcccatcatcctttgttctg
cas9 gene
(SEQ ID NO: 28)
atggactataaggaccacgacggagactacaaggatcatgatat
tgattacaaagacgatgacgataagatggccccaaagaagaagc
ggaaggtcggtatccacggagtcccagcagccgacaagaagtac
agcatcggcctggacatcggcaccaactctgtgggctgggccgt
gatcaccgacgagtacaaggtgcccagcaagaaattcaaggtgc
tgggcaacaccgaccggcacagcatcaagaagaacctgatcgga
gccctgctgttcgacagcggcgaaacagccgaggccacccggct
gaagagaaccgccagaagaagatacaccagacggaagaaccgga
tctgctatctgcaagagatcttcagcaacgagatggccaaggtg
gacgacagcttcttccacagactggaagagtccttcctggtgga
agaggataagaagcacgagcggcaccccatcttcggcaacatcg
tggacgaggtggcctaccacgagaagtaccccaccatctaccac
ctgagaaagaaactggtggacagcaccgacaaggccgacctgcg
gctgatctatctggccctggcccacatgatcaagttccggggcc
acttcctgatcgagggcgacctgaaccccgacaacagcgacgtg
gacaagctgttcatccagctggtgcagacctacaaccagctgtt
cgaggaaaaccccatcaacgccagcggcgtggacgccaaggcca
tcctgtctgccagactgagcaagagcagacggctggaaaatctg
atcgcccagctgcccggcgagaagaagaatggcctgttcggaaa
cctgattgccctgagcctgggcctgacccccaacttcaagagca
acttcgacctggccgaggatgccaaactgcagctgagcaaggac
acctacgacgacgacctggacaacctgctggcccagatcggcga
ccagtacgccgacctgtttctggccgccaagaacctgtccgacg
ccatcctgctgagcgacatcctgagagtgaacaccgagatcacc
aaggcccccctgagcgcctctatgatcaagagatacgacgagca
ccaccaggacctgaccctgctgaaagctctcgtgcggcagcagc
tgcctgagaagtacaaagagattttcttcgaccagagcaagaac
ggctacgccggctacattgacggcggagccagccaggaagagtt
ctacaagttcatcaagcccatcctggaaaagatggacggcaccg
aggaactgctcgtgaagctgaacagagaggacctgctgcggaag
cagcggaccttcgacaacggcagcatcccccaccagatccacct
gggagagctgcacgccattctgcggcggcaggaagatttttacc
cattcctgaaggacaaccgggaaaagatcgagaagatcctgacc
ttccgcatcccctactacgtgggccctctggccaggggaaacag
cagattcgcctggatgaccagaaagagcgaggaaaccatcaccc
cctggaacttcgaggaagtggtggacaagggcgcttccgcccag
agcttcatcgagcggatgaccaacttcgataagaacctgcccaa
cgagaaggtgctgcccaagcacagcctgctgtacgagtacttca
ccgtgtataacgagctgaccaaagtgaaatacgtgaccgaggga
atgagaaagcccgccttcctgagcggcgagcagaaaaaggccat
cgtggacctgctgttcaagaccaaccggaaagtgaccgtgaagc
agctgaaagaggactacttcaagaaaatcgagtgcttcgactcc
gtggaaatctccggcgtggaagatcggttcaacgcctccctggg
cacataccacgatctgctgaaaattatcaaggacaaggacttcc
tggacaatgaggaaaacgaggacattctggaagatatcgtgctg
accctgacactgtttgaggacagagagatgatcgaggaacggct
gaaaacctatgcccacctgttcgacgacaaagtgatgaagcagc
tgaagcggcggagatacaccggctggggcaggctgagccggaag
ctgatcaacggcatccgggacaagcagtccggcaagacaatcct
ggatttcctgaagtccgacggcttcgccaacagaaacttcatgc
agctgatccacgacgacagcctgacctttaaagaggacatccag
aaagcccaggtgtccggccagggcgatagcctgcacgagcacat
tgccaatctggccggcagccccgccattaagaagggcatcctgc
agacagtgaaggtggtggacgagctcgtgaaagtgatgggccgg
cacaagcccgagaacatcgtgatcgaaatggccagagagaacca
gaccacccagaagggacagaagaacagccgcgagagaatgaagc
ggatcgaagagggcatcaaagagctgggcagccagatcctgaaa
gaacaccccgtggaaaacacccagctgcagaacgagaagctgta
cctgtactacctgcagaatgggcgggatatgtacgtggaccagg
aactggacatcaaccggctgtccgactacgatgtggaccatatc
gtgcctcagagctttctgaaggacgactccatcgacaacaaggt
gctgaccagaagcgacaagaaccggggcaagagcgacaacgtgc
cctccgaagaggtcgtgaagaagatgaagaactactggcggcag
ctgctgaacgccaagctgattacccagagaaagttcgacaatct
gaccaaggccgagagaggcggcctgagcgaactggataaggccg
gcttcatcaagagacagctggtggaaacccggcagatcacaaag
cacgtggcacagatcctggactcccggatgaacactaagtacga
cgagaatgacaagctgatccgggaagtgaaagtgatcaccctga
agtccaagctggtgtccgatttccggaaggatttccagttttac
aaagtgcgcgagatcaacaactaccaccacgcccacgacgccta
cctgaacgccgtcgtgggaaccgccctgatcaaaaagtacccta
agctggaaagcgagttcgtgtacggcgactacaaggtgtacgac
gtgcggaagatgatcgccaagagcgagcaggaaatcggcaaggc
taccgccaagtacttcttctacagcaacatcatgaactttttca
agaccgagattaccctggccaacggcgagatccggaagcggcct
ctgatcgagacaaacggcgaaaccggggagatcgtgtgggataa
gggccgggattttgccaccgtgcggaaagtgctgagcatgcccc
aagtgaatatcgtgaaaaagaccgaggtgcagacaggcggcttc
agcaaagagtctatcctgcccaagaggaacagcgataagctgat
cgccagaaagaaggactgggaccctaagaagtacggcggcttcg
acagccccaccgtggcctattctgtgctggtggtggccaaagtg
gaaaagggcaagtccaagaaactgaagagtgtgaaagagctgct
ggggatcaccatcatggaaagaagcagcttcgagaagaatccca
tcgactttctggaagccaagggctacaaagaagtgaaaaaggac
ctgatcatcaagctgcctaagtactccctgttcgagctggaaaa
cggccggaagagaatgctggcctctgccggcgaactgcagaagg
gaaacgaactggccctgccctccaaatatgtgaacttcctgtac
ctggccagccactatgagaagctgaagggctcccccgaggataa
tgagcagaaacagctgtttgtggaacagcacaagcactacctgg
acgagatcatcgagcagatcagcgagttctccaagagagtgatc
ctggccgacgctaatctggacaaagtgctgtccgcctacaacaa
gcaccgggataagcccatcagagagcaggccgagaatatcatcc
acctgtttaccctgaccaatctgggagcccctgccgccttcaag
tactttgacaccaccatcgaccggaagaggtacaccagcaccaa
agaggtgctggacgccaccctgatccaccagagcatcaccggcc
tgtacgagacacggatcgacctgtctcagctgggaggcgacaaa
aggccggcggccacgaaaaaggccggccaggcaaaaaagaaaaa
gtaa
FtoS sgRNA2.0
(SEQ ID NO: 29)
gggcgtcctttattgtacttgttttagagctaggccaacatgag
gatcacccatgtctgcagggcctagcaagttaaaataaggctag
tccgttatcaacttggccaacatgaggatcacccatgtctgcag
ggccaagtggcaccgagtcggtgct
eGFP sgRNA2.0
(SEQ ID NO: 30)
agcgggagaagtttgcctgggttttagagctaggccaacatgag
gatcacccatgtctgcagggcctagcaagttaaaataaggctag
tccgttatcaacttggccaacatgaggatcacccatgtctgcag
ggccaagtggcaccgagtcggtgct
TF6 cassette for introduction of knock out
mutation in human topoisomerase I gene
(SEQ ID NO: 31)
ccacgtcatcctataaaagtgaaagaagtgcgctgctgtagtat
taccagcgcacttcggcagcggcagcacctcggcagcgtcagtg
aacaatgccaagcaagaaaagcatctattttgagtactttcttg
ctgtcttccagaaaatcaaagaggagaatgaaaaattactgaaa
gaatatggattctgtattatggataaccacaaagagaggattgc
taacttcaagatagagcctcctggactttagcgtggccgcggca
accaccccaagatgggcatgctgaagagacgaatcatgcccgag
gatataatcatcaactgtagcaagcgcagggaggcaaacaatga
tatcacaactctcctgacgcgtcatcgtcggctacagcctcggg
aattgctacctagctcgagcaagatccaaggagatataacaatg
gcttcctcctggatgattgaacaagatggattgcacgcaggttc
tccggccgcttgggtggagaggctattcggctatgactgggcac
aacagacaatcggctgctctgatgccgccgtgttccggctgtca
gcgcaggggcgcccggttctttttgtcaagaccgacctgtccgg
tgccctgaatgaactgcaggacgaggcagcgcggctatcgtggc
tggccacgacgggcgttccttgcgcagctgtgctcgacgttgtc
actgaagcgggaagggactggctgctattgggcgaagtgccggg
gcaggatctcctgtcatctcaccttgctcctgccgagaaagtat
ccatcatggctgatgcaatgcggcggctgcatacgcttgatccg
gctacctgcccattcgaccaccaagcgaaacatcgcatcgagcg
agcacgtactcggatggaagccggtcttgtcgatcaggatgatc
tggacgaagagcatcaggggctcgcgccagccgaactgttcgcc
aggctcaaggcgcgcatgcccgacggcgaggatctcgtcgtgac
ccatggcgatgcctgcttgccgaatatcatggtggaaaatggcc
gcttttctggattcatcgactgtggccggctgggtgtggcggac
cgctatcaggacatagcgttggctacccgtgatattgctgaaga
gcttggcggcgaatgggctgaccgcttcctcgtgctttacggta
tcgccgctcccgattcgcagcgcatcgccttctatcgccttctt
gacgagttcttctgacagacatgataagatacattgatgagttt
ggacaaaccacaactagaatgcagtgaaaaaaatgctttatttg
tgaaatttgtgatgctattgctttatttgtaaccattataagct
gcaataaacaagttaacaacaacaattgcattcattttatgttt
caggttcagggggaggtgtgggaggttttttaaagcaagtaaaa
cctctacaaatgtggtaccacgtcatcctataaaagtgaaagaa
gtgcgctgctgtagtattaccagcgcacttcggcagcggcagca
cctcggcagcgtcagtgaacaatgccaagcaagaaaagc
dCas9-PCV1 Rep
(SEQ ID NO: 32)
atggacaagaagtactccattgggctcgctatcggcacaaacag
cgtcggctgggccgtcattacggacgagtacaaggtgccgagca
aaaaattcaaagttctgggcaataccgatcgccacagcataaag
aagaacctcattggcgccctcctgttcgactccggggagacggc
cgaagccacgcggctcaaaagaacagcacggcgcagatataccc
gcagaaagaatcggatctgctacctgcaggagatctttagtaat
gagatggctaaggtggatgactctttcttccataggctggagga
gtcctttttggtggaggaggataaaaagcacgagcgccacccaa
tctttggcaatatcgtggacgaggtggcgtaccatgaaaagtac
ccaaccatatatcatctgaggaagaagcttgtagacagtactga
taaggctgacttgcggttgatctatctcgcgctggcgcatatga
tcaaatttcggggacacttcctcatcgagggggacctgaaccca
gacaacagcgatgtcgacaaactctttatccaactggttcagac
ttacaatcagcttttcgaagagaacccgatcaacgcatccggag
ttgacgccaaagcaatcctgagcgctaggctgtccaaatcccgg
cggctcgaaaacctcatcgcacagctccctggggagaagaagaa
cggcctgtttggtaatcttatcgccctgtcactcgggctgaccc
ccaactttaaatctaacttcgacctggccgaagatgccaagctt
caactgagcaaagacacctacgatgatgatctcgacaatctgct
ggcccagatcggcgaccagtacgcagacctttttttggcggcaa
agaacctgtcagacgccattctgctgagtgatattctgcgagtg
aacacggagatcaccaaagctccgctgagcgctagtatgatcaa
gcgctatgatgagcaccaccaagacttgactttgctgaaggccc
ttgtcagacagcaactgcctgagaagtacaaggaaattttcttc
gatcagtctaaaaatggctacgccggatacattgacggcggagc
aagccaggaggaattttacaaatttattaagcccatcttggaaa
aaatggacggcaccgaggagctgctggtaaagcttaacagagaa
gatctgttgcgcaaacagcgcactttcgacaatggaagcatccc
ccaccagattcacctgggcgaactgcacgctatcctcaggcggc
aagaggatttctacccctttttgaaagataacagggaaaagatt
gagaaaatcctcacatttcggataccctactatgtaggccccct
cgcccggggaaattccagattcgcgtggatgactcgcaaatcag
aagagaccatcactccctggaacttcgaggaagtcgtggataag
ggggcctctgcccagtccttcatcgaaaggatgactaactttga
taaaaatctgcctaacgaaaaggtgcttcctaaacactctctgc
tgtacgagtacttcacagtttataacgagctcaccaaggtcaaa
tacgtcacagaagggatgagaaagccagcattcctgtctggaga
gcagaagaaagctatcgtggacctcctcttcaagacgaaccgga
aagttaccgtgaaacagctcaaagaagactatttcaaaaagatt
gaatgtttcgactctgttgaaatcagcggagtggaggatcgctt
caacgcatccctgggaacgtatcacgatctcctgaaaatcatta
aagacaaggacttcctggacaatgaggagaacgaggacattctt
gaggacattgtcctcacccttacgttgtttgaagatagggagat
gattgaagaacgcttgaaaacttacgctcatctcttcgacgaca
aagtcatgaaacagctcaagaggcgccgatatacaggatggggg
cggctgtcaagaaaactgatcaatgggatccgagacaagcagag
tggaaagacaatcctggattttcttaagtccgatggatttgcca
acaggaacttcatgcagttgatccatgatgactctctcaccttt
aaggaggacatccagaaagcacaagtttctggccagggggacag
tcttcacgagcacatcgctaatcttgcaggtagcccagctatca
aaaagggaatactgcagaccgttaaggtcgtggatgaactcgtc
aaagtaatgggaaggcataagcccgagaatatcgttatcgagat
ggcccgagagaaccaaactacccagaagggacagaagaacagta
gggaaaggatgaagaggattgaagagggtataaaagaactgggg
tcccaaatccttaaggaacacccagttgaaaacacccagcttca
gaatgagaagctctacctgtactacctgcagaacggcagggaca
tgtacgtggatcaggaactggacatcaatcggctctccgactac
gacgtggatgctatcgtgccccagtcttttctcaaagatgattc
tattgataataaagtgttgacaagatccgataaaaatagaggga
agagtgataacgtcccctcagaagaagttgtcaagaaaatgaaa
aattattggcggcagctgctgaacgccaaactgatcacacaacg
gaagttcgataatctgactaaggctgaacgaggtggcctgtctg
agttggataaagccggcttcatcaaaaggcagcttgttgagaca
cgccagatcaccaagcacgtggcccaaattctcgattcacgcat
gaacaccaagtacgatgaaaatgacaaactgattcgagaggtga
aagttattactctgaagtctaagctggtctcagatttcagaaag
gactttcagttttataaggtgagagagatcaacaattaccacca
tgcgcatgatgcctacctgaatgcagtggtaggcactgcactta
tcaaaaaatatcccaagcttgaatctgaatttgtttacggagac
tataaagtgtacgatgttaggaaaatgatcgcaaagtctgagca
ggaaataggcaaggccaccgctaagtacttcttttacagcaata
ttatgaattttttcaagaccgagattacactggccaatggagag
attcggaagcgaccacttatcgaaacaaacggagaaacaggaga
aatcgtgtgggacaagggtagggatttcgcgacagtccggaagg
tcctgtccatgccgcaggtgaacatcgttaaaaagaccgaagta
cagaccggaggcttctccaaggaaagtatcctcccgaaaaggaa
cagcgacaagctgatcgcacgcaaaaaagattgggaccccaaga
aatacggcggattcgattctcctacagtcgcttacagtgtactg
gttgtggccaaagtggagaaagggaagtctaaaaaactcaaaag
cgtcaaggaactgctgggcatcacaatcatggagcgatcaagct
tcgaaaaaaaccccatcgactttctcgaggcgaaaggatataaa
gaggtcaaaaaagacctcatcattaagcttcccaagtactctct
ctttgagcttgaaaacggccggaaacgaatgctcgctagtgcgg
gcgagctgcagaaaggtaacgagctggcactgccctctaaatac
gttaatttcttgtatctggccagccactatgaaaagctcaaagg
gtctcccgaagataatgagcagaagcagctgttcgtggaacaac
acaaacactaccttgatgagatcatcgagcaaataagcgaattc
tccaaaagagtgatcctcgccgacgctaacctcgataaggtgct
ttctgcttacaataagcacagggataagcccatcagggagcagg
cagaaaacattatccacttgtttactctgaccaacttgggcgcg
cctgcagccttcaagtacttcgacaccaccatagacagaaagcg
gtacacctctacaaaggaggtcctggacgccacactgattcatc
agtcaattacggggctctatgaaacaagaatcgacctctctcag
ctcggtggagacgtgagcgctggaggaggtggaagcggaggagg
aggaagcggaggaggaggtagcctcgagaccatgccaagcaaga
aaagcggcccgcaaccccataagaggtgggtgttcacccttaat
aatccttccgaggaggagaaaaacaaaatacgggagcttccaat
ctccctttttgattattttgtttgcggagaggaaggtttggaag
agggtagaactcctcacctccaggggtttgcgaattttgctaag
aagcagacttttaacaaggtgaagtggtattttggtgcccgctg
ccacatcgagaaagcgaaaggaaccgaccagcagaataaagaat
attgcagtaaagaaggtcacatacttatcgagtgtggagccccc
cggaaccaggggaagcgcagcgacctgtctactgctgtgagtac
ccttttggagacggggtctttggtgactgtagccgagcagttcc
ctgtaacgtatgtgagaaatttccgggggctggctgaacttttg
aaagtgagcgggaagatgcagcagcgtgattggaagacagctgt
acacgtcatagtgggaccgcccggttgtgggaagagccagtggg
cccgtaattttactgagcctagcgacacctactggaagcctagt
agaaataagtggtgggatggctatcatggagaagaagttgttgt
tttggatgatttttatggctggttaccttgggatgatctactga
gactgtgtgaccggtatccattgactgtagagactaaaggcggt
actgttccttttttggcccgcagtattttgattaccagcaatca
ggccccccaggaatggtactcctcaactgctgtcccagccgtag
aagctctctatcggaggattactactttgcaattttggaagact
gctggagaacaatccacggaggtgcccgaaggccgatttgaagc
agtggacccaccctgtgcccttttcccatataaaataaattact
ag
TTVori-SERPINA1-TTVori
(SEQ ID NO: 33)
cggtgaagccacggagggagatctccgcgtcccgagggcgggtg
ccgaaggtgagtttacacaccgaagtcaaggggcaattcgggct
cgggactggccgggctatgggcaaggctctgagcaacagcaaca
aagcgcagccatttctttctgtttgcacagctcctctgtctgtc
gggggctcctgtctgttgtctcctataagcctcaccacctctcc
tactgcttgggcatgcatctttctccccttctatagatgaggag
gttaaggtccagagaggggtggggaggaacgccggctcacattc
tccatcccctccagatatgaccaggaacagacctgtgccaggcc
tcagccttacatcaaaatgggcctccccatgcaccgtggacctc
tgggccctcctgtcccagtggaggacaggaagctgtgaggggca
ctgtcacccagggctcaagctggcattcctgaataatcgctctg
caccaggccacggctaagctcagtgcgtgattaagcctcataac
cctccaaggcagttactagtgtgattcccattttacagatgagg
aagatggggacagagaggtgaataactggccccaaatcacacac
catccataattcgggctcaggcacctggctccagtccccaaact
cttgaacctggccctagtgtcactgtttctcttgggtctcaggc
gctggatggggaacaggaaacctgggctggacttgaggcctctc
tgatgctcggtgacttcagacagttgctcaacctctctgttctc
ttgggcaaaacatgataacctttgacttctgtcccctcccctca
ccccacccgaccttgatctctgaagtgttggaaggatttaattt
ttcctgcactgagttttggagacaggtcaaaaagatgaccaagg
ccaaggtggccagtttcctatagaacgcctctaaaagacctgca
gcaatagcagcaagaactggtattctcgagaacttgctgcgcag
caggcacttcttggcattttatgtgtatttaatttcacaatagc
tctatgacaaagtccacctttctcatctccaggaaactgaggtt
cagagaggttaagtaacttgtccaaggtcacacagctaatagca
agttgacgtggagcaatctggcctcagagcctttaattttagcc
acagactgatgctcccctcttcatttagccaggctgcctctgaa
gttttctgattcaagacttctggcttcagctttgtacacagaga
tgattcaatgtcaggttttggagtgaaatctgtttaatcccaga
caaaacatttaggattacatctcagttttgtaagcaagtagctc
tgtgatttttagtgagttatttaatgctctttggggctcaattt
ttctatctataaaatagggctaataatttgcaccttatagggta
agctttgaggacagattagatgatacggtgcctgtaaaacacca
ggtgttagtaagtgtggcaatgatggtgacgctgaggctgatgt
ttgcttagcatagggttaggcagctggcaggcagtaaacagttg
gataatttaatggaaaatttgccaaactcagatgctgttcactg
ctgagcaggagccccttcctgctgaaatggtcctggggagtgca
gcaggctctccgggaagaaatctaccatctctcgggcaggagct
caacctgtgtgcaggtacagggagggcttcctcacctggtgccc
actcatgcattacgtcagttattcctcatccctgtccaaaggat
tcttttctccattgtacagctatgaagctagtgctcaaagaagt
gaagtcatttaccccaggccccctgccagtaagtgacagggcct
ggtcacacttgggtttatttattgcccagttcaacaggttgttt
gaccataggcgagattctcttccctgcaccctgccgggttgctc
ttggtcccttattttatgctcccgggtagaaatggtgtgagatt
aggcagggagtggctcgcttccctgtccctggccccgcaaagag
tgctcccacctgccccgatcccagaaatgtcaccatgaagcctt
cattcttttggtttaaagcttggcctcagtgtccgtacaccatg
gggtacttggccagatggcgactttctcctctccagtcgccctc
ccaggcactagcttttaggagtgcagggtgctgcctctgataga
agggccaggagagagcaggttttggagtcctgatgttataagga
acagcttgggaggcataatgaacccaacatgatgcttgagacca
atgtcacagcccaattctgacattcatcatctgagatctgagga
cacagctgtctcagttcatgatctgagtgctgggaaagccaaga
cttgttccagctttgtcactgacttgctgtatagcctcaacaag
gccctgaccctctctgggcttcaaactcttcactgtgaaaggag
gaaaccagagtaggtgatgtgacaccaggaaagatggatgggtg
tgggggaatgtgctcctcccagctgtcaccccctcgccaccctc
cctgcaccagcctctccacctcctttgagcccagaattcccctg
tctaggagggcacctgtctcatgcctagccatgggaattctcca
tctgttttgctacattgaacccagatgccattctaaccaagaat
cctggctgggtgcaggggctctcgcctgtaaccccagcactttg
ggaggccaaggcaggcggatcaagaggtcaggagttcaagacct
gcctggccaacacggtgaaacctcagctctactaaaaatacaaa
aattagccaggcgtggtggcacacgcctgtaatcccagctattt
gggaagctgagacagaagaatttcttgaacccgggaggtggagg
tttcagtgagccgagatcacgccactgcactccaccctggcaga
taaagcgagactctgtctcaaaaaaaacccaaaaacctatgtta
gtgtacagagggccccagtgaagtcttctcccagccccactttg
cacaactggggagagtgaggccccaggaccagaggattcttgct
aaaggccaagtggatagtgatggccctgccagggctagaagcca
caacctctggccctgaggccactcagcatatttagtgtccccac
cctgcagaggcccaactccctcctgaccactgagccctgtaatg
atgggggaatttccataagccatgaaggactgcacaaagttcag
ttgggaagtgaaagagaaattaaagggagatggaaatatacagc
actaattttagcaccgtctttagttctaacaacactagctagct
gaagaaaaatacaaacatgtattatgtaatgtgtggtctgttcc
atttggattacttagaggcacgagggccaggagaaaggtggtgg
agagaaaccagctttgcacttcatttgttgctttattggaagga
aacttttaaaagtccaagggggttgaagaatctcaatatttgtt
atttccagctttttttctccagtttttcatttcccaaattcaag
gacacctttttctttgtattttgttaagatgatggttttggttt
tgtgactagtagttaacaatgtggctgccgggcatattctcctc
agctaggacctcagttttcccatctgtgaagacggcaggttcta
cctagggggctgcaggctggtggtccgaagcctgggcatatctg
gagtagaaggatcactgtggggcagggcaggttctgtgttgctg
tggatgacgttgactttgaccattgctcggcagagcctgctctc
gctggttcagccacaggccccaccactccctattgtctcagccc
cgggtatgaaacatgtattcctcactggcctatcacctgaagcc
tttgaatttgcaacacctgccaacccctccctcaaaagagttgc
cctctcagatccttttgatgtaaggtttggtgttgagacttatt
tcactaaattctcatacataaacatcactttatgtatgaggcaa
aatgaggaccagggagatgaatgacttgtcctggctcatacacc
tggaaagtgacagagtcagattagatcccaggtctatctgaagt
taaaagaggtgtcttttcacttcccacctcctccatctacttta
aagcagcacaaacccctgctttcaaggagagatgagcgtctcta
aagcccctgacagcaagagcccagaactgggacaccattagtga
cccagacggcaggtaagctgactgcaggagcatcagcctattct
tgtgtctgggaccacagagcattgtggggacagccccgtctctt
gggaaaaaaaccctaagggctgaggatccttgtgagtgttgggt
gggaacagctcccaggaggtttaatcacagcccctccatgctct
ctagctgttgccattgtgcaagatgcatttcccttctgtgcagc
agtttccctggccactaaatagtgggattagatagaagccctcc
aagggcttccagcttgacatgattcttgattctgatctggcccg
attcctggataatcgtgggcaggcccattcctcttcttgtgcct
cattttcttcttttgtaaaacaatggctgtaccatttgcatctt
agggtcattgcagatgtaagtgttgctgtccagagcctgggtgc
aggacctagatgtaggattctggttctgctacttcctcagtgac
attgaatagctgacctaatctctctggctttggtttcttcatct
gtaaaagaaggatattagcattagcacctcacgggattgttaca
agaaagcaatgaattaacacatgtgagcacggagaacagtgctt
ggcatatggtaagcactacgtacattttgctattcttctgattc
tttcagtgttactgatgtcggcaagtacttggcacaggctggtt
taataatccctaggcacttccacgtggtgtcaatccctgatcac
tgggagtcatcatgtgccttgactcggggcctggcccccccatc
tctgtcttgcaggacaatgccgtcttctgtctcgtggggcatcc
tcctgctggcaggcctgtgctgcctggtccctgtctccctggct
gaggatccccagggagatgctgcccagaagacagatacatccca
ccatgatcaggatcacccaaccttcaacaagatcacccccaacc
tggctgagttcgccttcagcctataccgccagctggcacaccag
tccaacagcaccaatatcttcttctccccagtgagcatcgctac
agcctttgcaatgctctccctggggaccaaggctgacactcacg
atgaaatcctggagggcctgaatttcaacctcacggagattccg
gaggctcagatccatgaaggcttccaggaactcctccgtaccct
caaccagccagacagccagctccagctgaccaccggcaatggcc
tgttcctcagcgagggcctgaagctagtggataagtttttggag
gatgttaaaaagttgtaccactcagaagccttcactgtcaactt
cggggacaccgaagaggccaagaaacagatcaacgattacgtgg
agaagggtactcaagggaaaattgtggatttggtcaaggagctt
gacagagacacagtttttgctctggtgaattacatcttctttaa
aggtaaggttgctcaaccagcctgagctgttcccatagaaacaa
gcaaaaatattctcaaaccatcagttcttgaactctccttggca
atgcattatgggccatagcaatgcttttcagcgtggattcttca
gttttctacacacaaacactaaaatgttttccatcattgagtaa
tttgaggaaataatagattaaactgtcaaaactactgacagctc
tgcagaacttttcagagcctttaatgtccttgtgtatactgtat
atgtagaatatataatgcttagaactatagaacaaattgtaata
cactgcataaagggatagtttcatggaacatactttacacgact
ctagtgtcccagaatcagtatcagttttgcaatctgaaagacct
gggttcaaatcctgcctctaacacaattagcttttgacaaaaac
aatgcattctacctctttgaggtgctaatttctcatcttagcat
ggacaaaataccattcttgctgtcaggtttttttaggattaaac
aaatgacaaagactgtggggatggtgtgtggcatacagcaggtg
atggactcttctgtatctcaggctgccttcctgcccctgagggg
ttaaaatgccagggtcctgggggccccagggcattctaagccag
ctcccactgtcccaggaaaacagcataggggaggggaggtggga
ggcaaggccaggggctgcttcctccactctgaggctcccttgct
cttgaggcaaaggagggcagtggagagcagccaggctgcagtca
gcacagctaaagtcctggctctgctgtggccttagtgggggccc
aggtccctctccagccccagtctcctccttctgtccaatgagaa
agctgggatcaggggtccctgaggcccctgtccactctgcatgc
ctcgatggtgaagctctgttggtatggcagaggggaggctgctc
aggcatctgcatttcccctgccaatctagaggatgaggaaagct
ctcaggaatagtaagcagaatgtttgccctggatgaataactga
gctgccaattaacaaggggcagggagccttagacagaaggtacc
aaatatgcctgatgctccaacattttatttgtaatatccaagac
accctcaaataaacatatgattccaataaaaatgcacagccacg
atggcatctcttagcctgacatcgccacgatgtagaaattctgc
atcttcctctagttttgaattatccccacacaatctttttcggc
agcttggatggtcagtttcagcaccttttacagatgatgaagct
gagcctcgagggatgtgtgtcgtcaagggggctcagggcttctc
agggaggggactcatggtttctttattctgctacactcttccaa
accttcactcacccctggtgatgcccaccttcccctctctccag
gcaaatgggagagaccctttgaagtcaaggacaccgaggaagag
gacttccacgtggaccaggtgaccaccgtgaaggtgcctatgat
gaagcgtttaggcatgtttaacatccagcactgtaagaagctgt
ccagctgggtgctgctgatgaaatacctgggcaatgccaccgcc
atcttcttcctgcctgatgaggggaaactacagcacctggaaaa
tgaactcacccacgatatcatcaccaagttcctggaaaatgaag
acagaaggtgattccccaacctgagggtgaccaagaagctgccc
acacctcttagccatgttgggactgaggcccatcaggactggcc
agagggctgaggagggtgaaccccacatccctgggtcactgcta
ctctgtataaacttggcttccagaatgaggccaccactgagttc
aggcagcgccatccatgctccatgaggaggacagtacccagggg
tgaggaggtaaaggtctcgtccctggggacttcccactccagtg
tggacactgtcccttcccaatatccagtgcccagggcagggaca
gcagcaccaccacacgttctggcagaaccaaaaaggaacagatg
ggcttcctggcaaaggcagcagtggagtgtggagttcaagggta
gaatgtccctggggggacgggggaagagcctgtgtggcaaggcc
cagaaaagcaaggttcggaattggaacagccaggccatgttcgc
agaaggcttgcgtttctctgtcactttatcggtgctgttagatt
gggtgtcctgtagtaagtgatacttaaacatgagccacacatta
gtgtatgtgtgtgcattcgtgattatgcccatgccctgctgatc
tagttcgttttgtacactgtaaaaccaagatgaaaatacaaaag
gtgtcgggttcataataggaatcgaggctggaatttctctgttc
catgccagcacctcctgaggtctctgctccaggggttgagaaag
aacaaagaggctgagagggtaacggatcagagagcccagagcca
agctgcccgctcacaccagaccctgctcagggtggcattgtctc
cccatggaaaaccagagaggagcactcagcctggtgtggtcact
cttctcttatccactaaacggttgtcactgggcactgccaccag
ccccgtgtttctctgggtgtagggccctggggatgttacaggct
gggggccaggtgacccaacactacagggcaagatgagacaggct
tccaggacacctagaatatcagaggaggtggcatttcaagcttt
tgtgattcattcgatgttaacattctttgactcaatgtagaaga
gctaaaagtagaacaaaccaaagccgagttcccatcttagtgtg
ggtggaggacacaggagtaagtggcagaaataatcagaaaagaa
aacacttgcactgtggtgggtcccagaagaacaagaggaatgct
gtgccatgccttgaatttcttttctgcacgacaggtctgccagc
ttacatttacccaaactgtccattactggaacctatgatctgaa
gagcgtcctgggtcaactgggcatcactaaggtcttcagcaatg
gggctgacctctccggggtcacagaggaggcacccctgaagctc
tccaaggtgagatcaccctgacgaccttgttgcaccctggtatc
tgtagggaagaatgtgtgggggctgcagctctgtcctgaggctg
aggaaggggccgagggaaacaaatgaagacccaggctgagctcc
tgaagatgcccgtgattcactgacacgggacgtggtcaaacagc
aaagccaggcaggggactgctgtgcagctggcactttcggggcc
tcccttgaggttgtgtcactgaccctgaatttcaactttgccca
agaccttctagacattgggccttgatttatccatactgacacag
aaaggtttgggctaagttgtttcaaaggaatttctgactccttc
gatctgtgagatttggtgtctgaattaatgaatgatttcagcta
aagatgacacttattttggaaaactaaaggcgaccaatgaacaa
ctgcagttccatgaatggctgcattatcttggggtctgggcact
gtgaaggtcactgccagggtccgtgtcctcaaggagcttcaagc
cgtgtactagaaaggagagagccctggaggcagacgtggagtga
cgatgctcttccctgttctgagttgtgggtgcacctgagcaggg
ggagaggcgcttgtcaggaagatggacagaggggagccagcccc
atcagccaaagccttgaggaggagcaaggcctatgtgacaggga
gggagaggatgtgcagggccagggccgtccagggggagtgagcg
cttcctgggaggtgtccacgtgagccttgctcgaggcctgggat
cagccttacaacgtgtctctgcttctctcccctccaggccgtgc
ataaggctgtgctgaccatcgacgagaaagggactgaagctgct
ggggccatgtttttagaggccatacccatgtctatcccccccga
ggtcaagttcaacaaaccctttgtcttcttaatgattgaacaaa
ataccaagtctcccctcttcatgggaaaagtggtgaatcccacc
caaaaataactgcctctcgctcctcaacccctcccctccatccc
tggccccctccctggatgacattaaagaagggttgagctggtcc
ctgcctgcatgtgactgtaaatccctcccatgttttctctgagt
ctccctttgcctgctgaggctgtatgtgggctccaggtaacagt
gctgtcttcgggccccctgaactgtgttcatggagcatctggct
gggtaggcacatgctgggcttgaatccaggggggactgaatcct
cagcttacggacctgggcccatctgtttctggagggctccagtc
ttccttgtcctgtcttggagtccccaagaaggaatcacagggga
ggaaccagataccagccatgaccccaggctccaccaagcatctt
catgtccccctgctcatcccccactcccccccacccagagttgc
tcatcctgccagggctggctgtgcccaccccaaggctgccctcc
tgggggccccagaactgcctgatcgtgccgtggcccagttttgt
ggcatctgcagcaacacaagagagaggacaatgtcctcctcttg
acccgctgtcacctaaccagactcgggccctgcacctctcaggc
acttctggaaaatgactgaggcagattcttcctgaagcccattc
tccatggggcaacaaggacacctattctgtccttgtccttccat
cgctgccccagaaagcctcacatatctccgtttagaatcaggtc
ccttctccccagatgaagaggagggtctctgctttgttttctct
atctcctcctcagacttgaccaggcccagcaggccccagaagac
cattaccctatatcccttctcctccctagtcacatggccatagg
cctgctgatggctcaggaaggccattgcaaggactcctcagcta
tgggagaggaagcacatcacccattgacccccgcaacccctccc
tttcctcctctgagtcccgactggggccacatgcagcctgacgg
tgaagccacggagggagatctccgcgtcccgagggcgggtgccg
aaggtgagtttacacaccgaagtcaaggggcaattcgggctcgg
gactggccgggctatgggcaaggctctga
CMV promoter
(SEQ ID NO: 34)
caatcccacaaaaatctgagcttaacagcacagttgctcctctc
agagcagaatcgggtattcaacaccctcatatcaactactacgt
tgtgtataacggtccacatgccggtatatacgatgactggggtt
gtacaaaggcggcaacaaacggcgttcccggagttgcacacaag
aaatttgccactattacagaggcaagagcagcagctgacgcgta
cacaacaagtcagcaaacagacaggttgaacttcatccccaaag
gagaagctcaactcaagcccaagagctttgctaaggccctaaca
agcccaccaaagcaaaaagcccactggctcacgctaggaaccaa
aaggcccagcagtgatccagccccaaaagagatctcctttgccc
cggagattacaatggacgatttcctctatctttacgatctagga
aggaagttcgaaggtgaagtagacgacactatgttcaccactga
taatgagaaggttagcctcttcaatttcagaaagaatgctgacc
cacagatggttagagaggcctacgcagcaggtctcatcaagacg
atctacccgagtaacaatctccaggagatcaaataccttcccaa
gaaggttaaagatgcagtcaaaagattcaggactaattgcatca
agaacacagagaaagacatatttctcaagatcagaagtactatt
ccagtatggacgattcaaggcttgcttcataaaccaaggcaagt
aatagagattggagtctctaaaaaggtagttcctactgaatcta
aggccatgcatggagtctaagattcaaatcgaggatctaacaga
actcgccgtgaagactggcgaacagttcatacagagtcttttac
gactcaatgacaagaagaaaatcttcgtcaacatggtggagcac
gacactctggtctactccaaaaatgtcaaagatacagtctcaga
agaccaaagggctattgagacttttcaacaaaggataatttcgg
gaaacctcctcggattccattgcccagctatctgtcacttcatc
gaaaggacagtagaaaaggaaggtggctcctacaaatgccatca
ttgcgataaaggaaaggctatcattcaagatctctctgccgaca
gtggtcccaaagatggacccccacccacgaggagcatcgtggaa
aaagaagacgttccaaccacgtcttcaaagcaagtggattgatg
tgacatctccactgacgtaagggatgacgcacaatcccactatc
cttcgcaagacccttcctctatataaggaagttcatttcatttg
gagaggacacg
human U6 promoter
(SEQ ID NO: 35)
tttcccatgattccttcatatttgcatatacgatacaaggctgt
tagagagataattggaattaatttgactgtaaacacaaagatat
tagtacaaaatacgtgacgtagaaagtaataatttcttgggtag
tttgcagttttaaaattatgttttaaaatggactatcatatgct
taccgtaacttgaaagtatttcgatttcttggctttatatatct
tgtggaaaggac

1. A nucleic acid encoding a first fusion protein comprising a 5′ to 3′ DNA exonuclease domain and an RNA binding domain.

2. A nucleic acid according to claim 1, wherein the exonuclease is λ-exo.

3. A nucleic acid according to claim 1, for inhibition of NHEJ during transformation of a cellular genome so as to promote HDR.

4. A nucleic acid according to any of claim 1, for transformation of a eukaryotic cell in concert with an RNA-guided endonuclease.

5. A nucleic acid composition comprising a nucleic acid according to claim 1 and a nucleic acid encoding a second fusion protein comprising an endonuclease domain and a binding domain for an origin of replication.

6. A nucleic acid composition comprising a nucleic acid according to claim 1 and a nucleic acid encoding a third fusion protein comprising a recombination inducing domain and an RNA binding domain.

7. A nucleic acid composition comprising a nucleic acid according to claim 1 and a nucleic acid encoding a fourth fusion protein comprising a domain comprising an inhibitor of the mismatch repair pathway; and an RNA binding domain.

8. A nucleic acid composition comprising a nucleic acid according to claim 1 and a nucleic acid encoding a fifth fusion protein comprising a Holliday junction resolvase domain and an RNA binding domain.

9. A nucleic acid according to claim 1, wherein the RNA binding domain binds to the RNA component of an RNA guided endonuclease for use in transformation mediated by the RNA-guided endonuclease.

10. A method of transforming the genome of a human or animal cell comprising the steps of:
a. expressing an RNA-guided endonuclease in the cell or introducing the RNA-guided endonuclease into the cell;
b. expressing in the cell or introducing into the cell a sequence specific guide RNA to direct cleavage by the endonuclease domain to a specific locus
c. expressing in the cell the nucleic acid of claim 1 or introducing into the cell the first fusion protein of claim 1.

11. A method according to claim 10, wherein the exonuclease is a dsDNA exonuclease.

12. A method according to claim 10 for modifying a genome, wherein a double strand break is introduced into the genome in the presence of an exogenous donor nucleic acid sequence comprising a donor nucleic acid sequence as a template for modifying the chromosome or as an exogenous sequence to be integrated into the chromosome, and a DNA repair mechanism modifies the genome via homology-directed repair (HDR), the method comprising:
suppressing non-homologous end joining (NHEJ) repair of the break to promote repair by HDR by expressing in the cell the nucleic acid of claim 1 or introducing into the cell the first fusion protein of claim 1.

13. A method according to claim 1013, 11 or 12, further comprising the steps of:
a. expressing in the cell the nucleic acid of claim 1; or
b. introducing into the cell the fusion protein of claim 1.

14. A method according to claim 13, comprising expressing in the cell a nucleic acid of claim 1 or introducing into the cell a fusion protein of claim 1, wherein the RNA binding protein domains of the respective fusion proteins bind to different RNA sequences.

15. (canceled)

16. Use a nucleic acid according to claim 1 in transformation of a human or animal cell using an RNA-guided endonuclease.

17-24. (canceled)

25. A nucleic acid according to claim 6, wherein the RNA binding domain binds to the RNA component of an RNA guided endonuclease for use in transformation mediated by the RNA-guided endonuclease.

26. A nucleic acid according to claim 7, wherein the RNA binding domain binds to the RNA component of an RNA guided endonuclease for use in transformation mediated by the RNA-guided endonuclease.

27. A nucleic acid according to claim 8, wherein the RNA binding domain binds to the RNA component of an RNA guided endonuclease for use in transformation mediated by the RNA-guided endonuclease.

28. A method according to claim 13, wherein the binding domain for an origin of replication of the second fusion protein binds to the origin of replication of the donor nucleic acid.

29. Use a nucleic acid according to claim 5 in transformation of a human or animal cell using an RNA-guided endonuclease.


SEQUENCE LISTING (35 sequences)

SEQ ID NO: 1 - DNA, 34 bp, Artificial Sequence
  MS2-derived stem-loop for binding
ggccaacatgaggatcacccatgtctgcagggcc

SEQ ID NO: 2 - DNA, 385 bp, Bacteriophage MS2
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggagatcctacctgaacatggaactcactatcccaattttcgctaccaattctgactgtgaactcatcgtgaaggcaatgcaggggctcctcaaagacggcaatcctatcccttccgccatcgccgctaactcaggca

SEQ ID NO: 3 - DNA, 25 bp, Artificial Sequence
  PP7-derived stem-loop for binding
taaggagtttatatggaaaccctta

SEQ ID NO: 4 - DNA, 387 bp, Bacteriophage PP7
atgtccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgagtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgttaa

SEQ ID NO: 5 - DNA, 17 bp, Artificial Sequence
  P22-derived stem-loop for binding B-box
accgccgacaacgcggt

SEQ ID NO: 6 - DNA, 303 bp, Bacteriophage P22
atgacggttatcacctacgggaagtcaacgtttgcaggcaatgctaaaactcgccgtcatgagcggcgcagaaagctagccatagagcgcgacaccatctgcaatatcatcgattcaatttttggctgcgatgctcctgatgcttctcaggaagttaaagccaaaagaattgaccgtgtcaccaaagccatttcgcttgccggaacgcgtcagaaggaagttgaaggaggatctgtacttcttccaggcgtagcactttacgcggctggtcatcgtaagagcaaacaaataacagcgaggtaa

SEQ ID NO: 7 - DNA, 167 bp, Artificial Sequence
  sgRNA2.0 with MS2 hairpins for modification of topoisomerase I gene
gatttacaacaaaacccagcgttttagagctaggccaacatgaggatcacccatgtctgcagggcctagcaagttaaaataaggctagtccgttatcaacttggccaacatgaggatcacccatgtctgcagggccaagtggcaccgagtcggtgcttttttttttt

SEQ ID NO: 8 - DNA, 155 bp, Artificial Sequence
  sgRNA comprising multiple stem-loops
gttttagagctaggccaacatgaggatcacccatgtctgcagggcctagcaagttaaaataaggctagtccgttatcaactttaaggagtttatatggaaacccttaaagtggcaccgagtcggtgctaccgccgacaacgcggttttttttttt

SEQ ID NO: 9 - DNA, 681 bp, Bacteriophage lambda
atgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatga

SEQ ID NO: 10 - DNA, 4140 bp, Streptococcus pyogenes
atggacaagaagtacagcatcggcctggacatcggcacgaactcggtgggctgggcggtgatcacggacgagtacaaggtgccctccaagaagttcaaggtgctgggcaacaccgaccgccactcgatcaagaagaacctgatcggcgccctgctgttcgactccggcgagaccgccgaggcgacgcgcctgaagcgcaccgcgcgtcgccgctacacgcgtcgcaagaaccgcatctgctacctgcaggagatcttcagcaacgagatggccaaggtggacgactcgttcttccaccgcctggaggagtccttcctggtggaggaagacaagaagcacgagcgccaccccatcttcggcaacatcgtggacgaggtggcctaccacgagaagtacccgacgatctaccacctgcgcaagaagctggtggacagcaccgacaaggcggacctgcgcctgatctacctggccctggcgcacatgatcaagttccgcggccacttcctgatcgagggcgacctgaaccccgacaactcggacgtggacaagctgttcatccagctggtgcagacctacaaccagctgttcgaggagaacccgatcaacgcctccggcgtggacgccaaggcgatcctgagcgcgcgcctgtccaagagccgtcgcctggagaacctgatcgcccagctgcccggcgagaagaagaacggcctgttcggcaacctgatcgcgctgtcgctgggcctgacgccgaacttcaagtccaacttcgacctggccgaggacgcgaagctgcagctgagcaaggacacctacgacgacgacctggacaacctgctggcccagatcggcgaccagtacgcggacctgttcctggccgcgaagaacctgtcggacgccatcctgctgtccgacatcctgcgcgtgaacaccgagatcacgaaggcccccctgtcggcgtccatgatcaagcgctacgacgagcaccaccaggacctgaccctgctgaaggcgctggtgcgccagcagctgccggagaagtacaaggagatcttcttcgaccagagcaagaacggctacgccggctacatcgacggcggcgcgtcgcaagaggagttctacaagttcatcaagcccatcctggagaagatggacggcacggaggagctgctggtgaagctgaaccgcgaggacctgctgcgcaagcagcgcaccttcgacaacggcagcatcccccaccagatccacctgggcgagctgcacgccatcctgcgtcgccaagaggacttctacccgttcctgaaggacaaccgcgagaagatcgagaagatcctgacgttccgcatcccctactacgtgggcccgctggcccgcggcaacagccgcttcgcgtggatgacccgcaagtcggaggagaccatcacgccctggaacttcgaggaagtggtggacaagggcgccagcgcgcagtcgttcatcgagcgcatgaccaacttcgacaagaacctgcccaacgagaaggtgctgccgaagcactccctgctgtacgagtacttcaccgtgtacaacgagctgacgaaggtgaagtacgtgaccgagggcatgcgcaagcccgccttcctgagcggcgagcagaagaaggcgatcgtggacctgctgttcaagaccaaccgcaaggtgacggtgaagcagctgaaagaggactacttcaagaagatcgagtgcttcgacagcgtggagatctcgggcgtggaggaccgcttcaacgccagcctgggcacctaccacgacctgctgaagatcatcaaggacaaggacttcctggacaacgaggagaacgaggacatcctggaggacatcgtgctgaccctgacgctgttcgaggaccgcgagatgatcgaggagcgcctgaagacgtacgcccacctgttcgacgacaaggtgatgaagcagctgaagcgtcgccgctacaccggctggggccgcctgagccgcaagctgatcaacggcatccgcgacaagcagtccggcaagaccatcctggacttcctgaagagcgacggcttcgcgaaccgcaacttcatgcagctgatccacgacgactcgctgaccttcaaagaggacatccagaaggcccaggtgtcgggccagggcgactccctgcacgagcacatcgccaacctggcgggctcccccgcgatcaagaagggcatcctgcagaccgtgaaggtggtggacgagctggtgaaggtgatgggccgccacaagccggagaacatcgtgatcgagatggcccgcgagaaccagaccacgcagaagggccagaagaacagccgcgagcgcatgaagcgcatcgaggaaggcatcaaggagctgggctcgcagatcctgaaggagcaccccgtggagaacacccagctgcagaacgagaagctgtacctgtactacctgcagaacggccgcgacatgtacgtggaccaggagctggacatcaaccgcctgtccgactacgacgtggaccacatcgtgccccagagcttcctgaaggacgactcgatcgacaacaaggtgctgacccgcagcgacaagaaccgcggcaagagcgacaacgtgccgtcggaggaagtggtgaagaagatgaagaactactggcgccagctgctgaacgccaagctgatcacgcagcgcaagttcgacaacctgaccaaggccgagcgcggtggcctgtcggagctggacaaggcgggcttcatcaagcgccagctggtggagacccgccagatcacgaagcacgtggcgcagatcctggactcccgcatgaacacgaagtacgacgagaacgacaagctgatccgcgaggtgaaggtgatcaccctgaagtccaagctggtcagcgacttccgcaaggacttccagttctacaaggtgcgcgagatcaacaactaccaccacgcccacgacgcgtacctgaacgccgtggtgggcaccgcgctgatcaagaagtaccccaagctggagagcgagttcgtgtacggcgactacaaggtgtacgacgtgcgcaagatgatcgccaagtcggagcaggagatcggcaaggccaccgcgaagtacttcttctactccaacatcatgaacttcttcaagaccgagatcacgctggccaacggcgagatccgcaagcgcccgctgatcgagaccaacggcgagacgggcgagatcgtgtgggacaagggccgcgacttcgcgaccgtgcgcaaggtgctgagcatgccccaggtgaacatcgtgaagaagaccgaggtgcagacgggcggcttctccaaggagagcatcctgccgaagcgcaactcggacaagctgatcgcccgcaagaaggactgggaccccaagaagtacggcggcttcgactccccgaccgtggcctacagcgtgctggtggtggcgaaggtggagaagggcaagtccaagaagctgaagagcgtgaaggagctgctgggcatcaccatcatggagcgcagctcgttcgagaagaaccccatcgacttcctggaggccaagggctacaaagaggtgaagaaggacctgatcatcaagctgccgaagtactcgctgttcgagctggagaacggccgcaagcgcatgctggcctccgcgggcgagctgcagaagggcaacgagctggccctgcccagcaagtacgtgaacttcctgtacctggcgtcccactacgagaagctgaagggctcgccggaggacaacgagcagaagcagctgttcgtggagcagcacaagcactacctggacgagatcatcgagcagatctcggagttctccaagcgcgtgatcctggccgacgcgaacctggacaaggtgctgagcgcctacaacaagcaccgcgacaagcccatccgcgagcaggcggagaacatcatccacctgttcaccctgacgaacctgggcgccccggccgcgttcaagtacttcgacaccacgatcgaccgcaagcgctacacctccacgaaagaggtgctggacgcgaccctgatccaccagagcatcaccggcctgtacgagacgcgcatcgacctgagccagctgggcggcgactcccgcgcggacccgaagaagaagcgcaaggtgtaa

SEQ ID NO: 11 - DNA, 5271 bp, Artificial Sequence
  cas9-B-PCV1 rep fusion protein gene
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggtgagcgctggaggaggtggaagcggaggaggaggaagcggaggaggaggtagcctcgagaccatgccaagcaagaaaagcggcccgcaaccccataagaggtgggtgttcacccttaataatccttccgaggaggagaaaaacaaaatacgggagcttccaatctccctttttgattattttgtttgcggagaggaaggtttggaagagggtagaactcctcacctccaggggtttgcgaattttgctaagaagcagacttttaacaaggtgaagtggtattttggtgcccgctgccacatcgagaaagcgaaaggaaccgaccagcagaataaagaatattgcagtaaagaaggtcacatacttatcgagtgtggagccccccggaaccaggggaagcgcagcgacctgtctactgctgtgagtacccttttggagacggggtctttggtgactgtagccgagcagttccctgtaacgtatgtgagaaatttccgggggctggctgaacttttgaaagtgagcgggaagatgcagcagcgtgattggaagacagctgtacacgtcatagtgggaccgcccggttgtgggaagagccagtgggcccgtaattttactgagcctagcgacacctactggaagcctagtagaaataagtggtgggatggctatcatggagaagaagttgttgttttggatgatttttatggctggttaccttgggatgatctactgagactgtgtgaccggtatccattgactgtagagactaaaggcggtactgttccttttttggcccgcagtattttgattaccagcaatcaggccccccaggaatggtactcctcaactgctgtcccagccgtagaagctctctatcggaggattactactttgcaattttggaagactgctggagaacaatccacggaggtgcccgaaggccgatttgaagcagtggacccaccctgtgcccttttcccatataaaataaattactga

SEQ ID NO: 12 - DNA, 170 bp, Porcine circovirus
ctgcgggggcgggtccttcttctgcggtaacgcctccttggccacgtcatcctataaaagtgaaagaagtgcgctgctgtagtattaccagcgcacttcggcagcggcagcacctcggcagcgtcagtgaaaatgccaagcaagaaaagcggcccgcaaccccataagag

SEQ ID NO: 13 - DNA, 3237 bp, Artificial Sequence
  Triple fusion of E.coli recA gene
atggacgagaacaagaagcgcgccctggccgcggccctgggacagatcgaacgccaattcggcaaaggcgcggtcatgcgcatgggcgaccatgagcgccaggcgatcccggccatctccaccggctccctgggtctggacatcgccctcggcatcggcggcctgcccaagggccggatcgtcgagatctacggtccggaatcctcgggcaagaccaccctgaccctctcggtgatcgccgaggcccagaaacagggcgccacctgtgccttcgtcgacgccgagcacgcgctcgatcccgactatgccggcaagctgggcgtcaacgtcgacgacctgctggtctcccagccggacaccggcgagcaggccctggaaatcaccgacatgctggtgcgctccaacgcggtcgacgtgatcatcgtcgactccgtggccgcgctggtacccaaggccgagatcgaaggcgagatgggcgacgcccacgtcggcctgcaggcacgcctgatgtcccaggcgctgcgcaagatcaccggcaatatcaagaacgccaactgcctggtcatcttcatcaaccagatccgcatgaagatcggcgtcatgttcggcaacccggaaaccaccaccggcggtaacgcactgaagttctacgcctcggtccgcctggacatccgtcgtaccggcgcggtgaaggaaggcgacgaggtggtgggtagcgaaacccgcgtcaaggtggtgaagaacaaggtttccccgccgttccgccaggccgagttccagatcctctacggtaagggcatctaccgtaccggcgagatcatcgatctgggcgtgcaattgggcctggtcgagaagtccggcgcctggtacagctaccagggcagcaagatcggccagggcaaggcgaacgccgccaagtacctggaagacaatccggaaatcggttcggtactggagaagaccattcgcgaccagttgctggccaagagcggcccggtgaaggccgacgccgaagaagtggctgacgccgaagccgattcagagctcggagaaggtcaaggacagggacaaggtccaggacgaggatacgcatataagcttgacgagaacaagaagcgcgccctggccgcggccctgggacagatcgaacgccaattcggcaaaggcgcggtcatgcgcatgggcgaccatgagcgccaggcgatcccggccatctccaccggctccctgggtctggacatcgccctcggcatcggcggcctgcccaagggccggatcgtcgagatctacggtccggaatcctcgggcaagaccaccctgaccctctcggtgatcgccgaggcccagaaacagggcgccacctgtgccttcgtcgacgccgagcacgcgctcgatcccgactatgccggcaagctgggcgtcaacgtcgacgacctgctggtctcccagccggacaccggcgagcaggccctggaaatcaccgacatgctggtgcgctccaacgcggtcgacgtgatcatcgtcgactccgtggccgcgctggtacccaaggccgagatcgaaggcgagatgggcgacgcccacgtcggcctgcaggcacgcctgatgtcccaggcgctgcgcaagatcaccggcaatatcaagaacgccaactgcctggtcatcttcatcaaccagatccgcatgaagatcggcgtcatgttcggcaacccggaaaccaccaccggcggtaacgcactgaagttctacgcctcggtccgcctggacatccgtcgtaccggcgcggtgaaggaaggcgacgaggtggtgggtagcgaaacccgcgtcaaggtggtgaagaacaaggtttccccgccgttccgccaggccgagttccagatcctctacggtaagggcatctaccgtaccggcgagatcatcgatctgggcgtgcaattgggcctggtcgagaagtccggcgcctggtacagctaccagggcagcaagatcggccagggcaaggcgaacgccgccaagtacctggaagacaatccggaaatcggttcggtactggagaagaccattcgcgaccagttgctggccaagagcggcccggtgaaggccgacgccgaagaagtggctgacgccgaagccgattcaggatccggagaaggtcaaggacagggacaaggtccaggacgaggatacgcatatgcatgcgacgagaacaagaagcgcgccctggccgcggccctgggacagatcgaacgccaattcggcaaaggcgcggtcatgcgcatgggcgaccatgagcgccaggcgatcccggccatctccaccggctccctgggtctggacatcgccctcggcatcggcggcctgcccaagggccggatcgtcgagatctacggtccggaatcctcgggcaagaccaccctgaccctctcggtgatcgccgaggcccagaaacagggcgccacctgtgccttcgtcgacgccgagcacgcgctcgatcccgactatgccggcaagctgggcgtcaacgtcgacgacctgctggtctcccagccggacaccggcgagcaggccctggaaatcaccgacatgctggtgcgctccaacgcggtcgacgtgatcatcgtcgactccgtggccgcgctggtacccaaggccgagatcgaaggcgagatgggcgacgcccacgtcggcctgcaggcacgcctgatgtcccaggcgctgcgcaagatcaccggcaatatcaagaacgccaactgcctggtcatcttcatcaaccagatccgcatgaagatcggcgtcatgttcggcaacccggaaaccaccaccggcggtaacgcactgaagttctacgcctcggtccgcctggacatccgtcgtaccggcgcggtgaaggaaggcgacgaggtggtgggtagcgaaacccgcgtcaaggtggtgaagaacaaggtttccccgccgttccgccaggccgagttccagatcctctacggtaagggcatctaccgtaccggcgagatcatcgatctgggcgtgcaattgggcctggtcgagaagtccggcgcctggtacagctaccagggcagcaagatcggccagggcaaggcgaacgccgccaagtacctggaagacaatccggaaatcggttcggtactggagaagaccattcgcgaccagttgctggccaagagcggcccggtgaaggccgacgccgaagaagtggctgacgccgaagccgattaa

SEQ ID NO: 14 - DNA, 3174 bp, Homo sapiens
atggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagacgagctcggagaaggtcaaggacagggacaaggtccaggacgaggatacgcatataagcttatggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagacggatccggagaaggtcaaggacagggacaaggtccaggacgaggatacgcatatgcatgcatggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagactga

SEQ ID NO: 15 - DNA, 2775 bp, Saccharomyces cerevisiae
atggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactccgggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagaacctcagagtgacagagccaaaagaccagtgcctcattttgctgacatggaaaaggaaacttcgtgggggaaagagatctgcttgcagtcggccagagagacagaaccagggcagtgcgactccaagtgcatcagcatag

SEQ ID NO: 16 - DNA, 3729 bp, Saccharomyces cerevisiae
atggccccagctacccctaaaacttctaagactgcacacttcgaaaatggctccacatcttctcaaaagaaaatgaagcaatcgagtttgttatcttttttctcaaaacaggtaccttctggcacaccgtcaaagaaggtccagaagcctactccagcgactttagaaaatacagctactgataagataacaaagaatccacaaggaggaaagacaggcaaacttttcgtagatgtagacgaagacaatgatttgacaatagctgaagaaacggtatcaactgtaaggagtgacataatgcattctcaagagccccaatctgatactatgttaaatagtaacacgacagaacctaaaagtaccactacggacgaggatttatcatcttcccaatctcgaaggaatcacaaaaggagagtgaattatgccgaaagtgatgatgatgactcagatactacattcactgctaaacggaagaagggtaaggtagttgatagtgaaagcgatgaggatgaatatttaccagataaaaatgacggcgatgaagatgacgatattgcagacgacaaagaggacatcaaaggagaattggcagaagatagtggtgatgacgatgatttaatctctctggcagaaacgacttctaagaaaaaattttcatacaacacttcgcattcttcatcgccctttacaagaaatatatcccgtgacaactcaaaaaagaaaagcaggccaaatcaggcaccaagtagatcgtacaatccttctcatagtcaaccatcagcgacttctaagtctagcaaattcaataaacaaaatgaagaacgatatcaatggttagtggatgaacgagatgctcagcgccgtcccaagagtgatccagagtacgatccaagaacactgtacatcccatcttctgcatggaacaagtttactccgtttgaaaaacaatattgggaaattaaatccaaaatgtgggattgtattgtctttttcaaaaagggtaaggcctttgaattatatgaaaaggatgcattattggctaatgcattatttgacttaaagattgcaggtggaggacgcgctaatatgcaactagctgggattccagagatgtcatttgaatattgggccgctcagtttatccaaatgggatataaagttgcgaaagtggatcaaagagagtcaatgttggctaaagaaatgagggaaggttctaaaggtatcgttaaaagagaactccagtgcatattgacatcgggtacgttaactgatggagatatgttgcattcggatctggctactttttgtcttgccatcagggaagaacctggtaacttctacaatgaaactcagctagattcatcaacgatagtgcaaaagctgaatacaaaaatattcggtgcagcattcattgatactgcaactggtgagcttcaaatgctggagtttgaagatgatagtgaatgcaccaaattggacacattaatgtcgcaagtaagacctatggaagtcgtcatggagaggaataacttaagcacactagctaacaaaattgtaaaattcaattcagcacccaatgcaatttttaatgaggttaaagccggtgaagaattttatgactgcgataaaacctatgctgaaatcatatcatcagaatacttttctacggaagaagattggccagaggttttgaaatcctattatgatacgggtaaaaaagttggctttagtgcatttggtggattattgtattatttgaagtggttaaaactggataagaatttaatttcgatgaaaaatatcaaagagtacgattttgtaaaatcgcaacactccatggttttagatggtatcactttacagaacttggaaatattttccaattcgtttgatggttctgataagggtactttgttcaaattattcaacagggctattactccaatgggtaagagaatgatgaaaaagtggttaatgcatccattattacgtaagaatgatattgaaagcaggctcgatagtgttgactctttgttacaagatattacattaagagaacagctagagataacattttcaaaattgccagatttggaaagaatgttggcccgtatccatagtaggacaattaaagtgaaagattttgaaaaggtaattaccgcttttgaaacgattattgaattacaagactctttaaagaacaacgatttgaagggagatgtttcaaaatacatatcctctttccctgagggacttgtggaagctgtcaaaagctggactaatgcttttgaaagacaaaaagcaatcaatgaaaatatcattgtaccacaaaggggatttgacatagagtttgataagtcaatggatagaatacaggagctagaagacgaattgatggaaattctgatgacttataggaagcaattcaaatgttccaatatacaatataaagattccggaaaggagatatacactattgaaattcctatctctgcaactaaaaatgtcccatcaaattgggttcaaatggctgcgaacaaaacgtacaagagatactactctgatgaagtaagagctttagcaagatcgatggcggaggctaaagaaatacacaagacgttagaggaggacctaaaaaatagattatgtcaaaaatttgatgcgcattataatacaatttggatgccaaccatacaggccatctctaacatagattgtttgttggcaattacaaggacatctgaatatctaggtgctccctcttgtaggccaaccattgtagatgaggtggactcgaaaacgaatactcaattaaatggttttctgaaatttaaatcattaagacatccatgctttaacctcggcgctactacagcaaaggacttcattcctaacgacattgaattaggtaaagaacagcccagattaggattgttaacgggtgccaacgcagctggtaaatccacgatattgaggatggcatgtattgccgtaattatggcacaaatgggatgttatgttccttgtgaatctgccgttctgacgccgattgatagaatcatgactcgtttaggcgctaatgataatatcatgcaaggtaaatctactttttttgtggaattggcggaaacaaaaaaaatattagacatggctaccaataggtcattattagttgttgacgagttgggaagaggaggttcatcgagtgatggttttgcaattgcagaaagtgtgctacatcatgtcgcgacacatattcaaagtctgggtttttttgccacacattatgggacattggcatcaagtttcaagcaccatcctcaagtaagaccactgaaaatgagcattctagtcgatgaagcgactaggaacgttacatttttgtataagatgcttgaaggacaaagtgaaggctcttttggtatgcatgttgcgtcaatgtgtggcatttcaaaggaaattattgataacgcgcagattgccgctgataatttagaacatacttctaggctagtcaaagaacgtgatttagctgcaaataatttgaatggtgaagttgtctctgtgccaggtggcttacaaagtgatttcgttcgaattgcgtacggagatggactgaagaatacaaaattaggttctggtgaaggggtcttgaactatgattggaacataaaaaggaacgttttgaaaagtctatttagtataattgatgatttacaatcctaa

SEQ ID NO: 17 - DNA, 474 bp, Bacteriophage T4
atgttattgactggcaaattatacaaagaagaaaaacagaaattttatgatgcacaaaacggtaaatgcttaatttgccaacgagaactaaatcctgatgttcaagctaatcacctcgaccatgaccatgaattaaatggaccaaaagcaggaaaggtgcgtggattgctttgtaatctatgcaatgctgcagaaggtcaaatgaagcataaatttaatcgttctggcttaaagggacaaggtgttgattatcttgaatggttagaaaatttacttacttatttaaaatccgattacacccaaaataatattcaccctaactttgttggagataaatcaaaggaattttctcgtttaggaaaagaggaaatgatggccgagatgcttcaaagaggatttgaatataatgaatctgacaccaaaacacaattaatagcttcattcaagaagcagcttagaaagagtttaaaatga

SEQ ID NO: 18 - DNA, 1062 bp, Saccharomyces cerevisiae
atgtcgacagcacagaaagctaagatattgcaactcatcgattcctgctgccaaaatgcaaaaagcacacaactgaaatctttatcatttgttattggagcagtaaatggcacgacgaaagaagctaaaagaacctacattcaagaacagtgtgaatttttggagaagttacgacaacaaaagataagagagggaagaattaacatattgtctatggatgctggtgtttctaactttgctttctctaagatgcaattgctcaataatgatccgctccctaaagtactagactggcaaaagataaatctagaggagaaattttttcaaaacctcaaaaagttaagcttgaatcctgctgaaacttctgagcttgtatttaaccttacggagtatttatttgaatctatgccgataccagatatgtttacaattgaaaggcaacgtaccagaactatgtcttcgaggcatattttagacccaattttaaaagtgaatattctcgaacagattcttttctctaacttggaaaataaaatgaagtatacgaataaaataccgaatacgtccaagttgaggtatatggtatgttcgtccgatccacatcggatgacttcatattggtgcattccaagagaagagacaccgaccagttcaaaaaagttaaaatctaacaaacatagcaaagattctcgaataaagctagtgaaaaaaatactttcaacctcaatactagaaggtaattcaactagttctacaaaactggtcgagttcataggagtttggaataataggataagaaatgcccttaccaaaaaaaaaagtttcaagctatgtgatatactagagatccaagataattcgggggtgagaaaagatgacgatttggcagattcattcctccattgtttgtcttggatggagtggttaaaaaattatgaaagtattactgaactcttgaattcaaaaacactggttaaaacacagttcggacaggtgtttgaattttgtgaaaataaggtacaaaagctgaaatttttgcagaacacttacaacaatgactaa

SEQ ID NO: 19 - DNA, 2727 bp, Homo sapiens
atgggagtgaatgacttgtggcaaattttggagcctgttaagcaacacatccccttgcgtaatcttggtgggaaaaccattgcagttgatctgagtctctgggtgtgtgaggcacagacagtcaaaaaaatgatgggcagcgtcatgaagccccacctcaggaacttattttttcgtatctcatatttaacacaaatggatgtaaaactggtatttgttatggaaggggaaccaccaaagctgaaagctgatgtcataagcaagaggaatcagtctcggtatgggtcttctggaaaatcgtggtctcagaaaacagggagatcacattttaaatcagtcttaagagagtgcctccatatgctcgaatgcttaggaatcccctgggttcaggctgctggggaagctgaagccatgtgtgcttatctcaatgctggtggtcatgtcgatggctgcctcaccaatgatggagatactttcctttatggggcccagactgtttacaggaatttcactatgaatacaaaggacccacatgttgactgttacacaatgtcatctatcaagagtaaactaggtttggatagagatgctctggttggattagcaatacttcttggctgtgattatctcccaaagggagtccctggagttggaaaagagcaagcattaaaacttatacagattttgaaagggcaaagtttacttcagaggtttaatcggtggaatgaaacatcttgtaactctagtccacaactgctagtcactaaaaaactggctcattgttccgtatgttcccatccaggttcacctaaggatcatgaacgtaatggatgcagattatgtaaaagtgataaatattgtgagccacatgactatgaatactgctgtccttgtgagtggcaccgtacagaacatgataggcaactcagtgaagtagagaacaatattaagaagaaagcttgctgttgtgagggattcccattccatgaggttattcaagaattccttttaaacaaggataaattggtgaaggttatcaggtaccaaagacctgatttgttattgtttcagagatttactcttgaaaaaatggagtggcccaatcactatgcatgtgagaaattgctggtacttttgacccattatgacatgatagaaagaaagcttggtagcagaaactctaatcaactacagccaattcgaattgttaagactcgaatcagaaatggagttcattgttttgaaatagaatgggaaaagcctgaacattatgctatggaagataaacaacatggagaatttgctttattaacaattgaggaagaatcattgtttgaagcagcatatcctgagatcgttgctgtttaccaaaaacaaaagttagaaattaaagggaagaaacaaaaacgtattaagcctaaagaaaacaatttgccagaaccagatgaagtaatgagctttcagtcacacatgactttaaaacccacatgtgaaatctttcataagcagaattccaagttaaattcggggatttcccctgatcctacattaccacaggaatctatttctgcctcattgaatagcttgcttttacctaaaaatactccatgtttgaatgcacaagaacagttcatgtcttctctaagacctttggctatacagcaaattaaagctgtcagtaagtctctaatttcagaatctagtcaacccaatacctcatctcataatatatccgtgattgctgatctacacttgagcactattgactgggaaggtacttcttttagtaattctccagctattcaaaggaatactttttctcatgatttaaaatcagaagttgaatcagagctatcagccatccctgatggctttgaaaatatcccagaacaactgtcctgtgaatcagaaaggtacactgcaaacataaagaaagtgttggatgaggattctgatgggattagtcctgaagagcatctactttctggcattactgatttatgtcttcaggatttgcctttaaaggaacgaatatttacaaaattatcatatcctcaggataatctacaaccagatgtcaacctgaaaactttgtccatacttagtgtaaaagaatcttgtattgctaacagtggttctgattgtacatcacatctttcaaaggatcttccaggaattcccttgcaaaatgaatccagagactctaaaattctaaaaggagaccagctgcttcaagaagactataaagtcaatacttctgtcccttattctgtcagtaacacagtggtaaagacctgcaatgttagaccaccaaatactgctttagatcatagtagaaaagttgatatgcaaaccactcggaaaattttaatgaagaagagtgtttgccttgacagacattcctctgatgaacaaagtgccccagtgtttgggaaagctaagtacacaactcaaagaatgaagcacagttctcaaaagcataattcatcccatttcaaagaaagtggccataacaagttgagtagccctaagatacatattaaagaaactgaacagtgtgtcagatcttatgaaacagctgaaaatgaagaaagctgtttcccagattcaacaaaaagttctctgagttctctacaatgtcataagaaagaaaacaactctggtacttgtttggatagccctcttcctttacgccagagattaaaactaagattccaaagcacttga

SEQ ID NO: 20 - DNA, 1800 bp, Arabidopsis thaliana
atgggtgtgggaggcaatttctgggatttgctgagaccatatgctcagcaacaaggctttgattttctcagaaacaaacgagtcgctgttgatctctccttctggatcgttcagcatgaaaccgctgttaagggtttcgtccttaaacctcacctccgactcactttcttccgtactatcaacctcttctcaaagtttggagcgtacccggtttttgtggttgatggaacaccatcacctttgaaatctcaggcgagaatctccaggtttttccgttcttctggaattgatacttgtaatctacctgtgattaaagatggtgtctcggttgagagaaacaagctgttttctgaatgggttagggaatgtgtggagctactcgaattgctcggtattccggtgctgaaagctaatggtgaggctgaagctctctgtgcacagttaaacagccaaggttttgtggatgcttgcattactcctgatagtgatgctttcctttttggtgctatgtgcgtgatcaaagacatcaagcctaattcaagagaaccttttgaatgctaccatatgtcacatatcgagtctggcctcggtttgaagcggaaacacttgattgctatttctctattggtgggaaacgattatgattcaggcggtgttcttgggattggtgtggataaagcactgcgcattgttcgtgagttttctgaagaccaagtacttgaaagactacaggacattggaaatgggttgcaacctgcagttcctggtggaatcaaatccggggatgatggtgaagaattccgctcagagatgaaaaaaagatctcctcactgttcccgttgtggacacctgggcagcaagagaactcattttaagtcctcttgtgagcactgcggttgtgatagtggttgcattaaaaaaccattagggtttagatgtgaatgctccttttgttccaaggatcgagatttaagggaacaaaagaaaaccaatgattggtggatcaaagtctgcgataagattgctctagcgccagagtttcccaacagaaagattattgaactttatctatccgatggtttgatgacaggagatggatcgtcaatgtcttggggaactcctgatactggaatgctagtggatctcatggttttcaaactgcactgggacccatcttatgttagaaaaatgttgcttccgatgctgtcgaccatttatctgagagaaaaggcaagaaacaacacaggatacgctttgttgtgtgatcaatacgaatttcattcaatcaagtgcataaaaactagatatgggcatcagtcctttgtaataaggtggagaaaacccaaatctacaagtggttatagtcatagtcacagcgagccagaagaatcaattgttgtattggaagaagaagaagagtctgttgatccgttggatggtttaaatgaacctcaggtgcaaaatgataatggtgactgcttcttgctaactgatgaatgcataggacttgttcagtctgctttccctgatgaaacagagcattttctacatgagaagaaactgagagagtcgaaaaagaagaatgtttctgaagaagaaacagcaacaccaagagcaacaacaatgggtgtacaaagaagcattaccgatttctaccgttcagcgaagaaagcagcagcaggtcaaagtatagagacaggcgggagttcaaaagcttctgcggaaaagaagagacaggcaacttctactagtagtagtaaccttacaaagtcggtcaggcgtcgtctcttgtttggatag

SEQ ID NO: 21 - DNA, 795 bp, Escherichia coli
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 22 - DNA, 600 bp, Streptomyces alboniger
atgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 23 - DNA, 222 bp, Simian virus 40
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 24 - DNA, 254 bp, Torque teno virus
cggtgaagccacggagggagatctccgcgtcccgagggcgggtgccgaaggtgagtttacacaccgaagtcaaggggcaattcgggctcgggactggccgggctatgggcaaggctctgaaaaaagcatgtttattggcaggcattacagaaagaaaagggcgctgtcactgtgtgctgtgcgaacaacaaagaaggcttgcaaactactaatagtaatgtggaccccacctcgcaatgatcaacactacctta

SEQ ID NO: 25 - DNA, 1161 bp, Bacteriophage lambda
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggagatcctacctgaacatggaactcactatcccaattttcgctaccaattctgactgtgaactcatcgtgaaggcaatgcaggggctcctcaaagacggcaatcctatcccttccgccatcgccgctaactcaggcatctacagcgctggaggaggtggaagcggaggaggaggaagcggaggaggaggtagcggacctaagaaaaagaggaaggtggctgccgctggatccatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatga

SEQ ID NO: 26 - DNA, 1501 bp, Artificial Sequence
  Topoisomerase I donor gene - camptothecin sensitive mutant
gtccggcagagagctgtagccctgtacttcatcgacaagcttgctctgagagcaggcaatgaaaaggaggaaggagaaacagcggacactgtgggctgctgctcacttcgtgtggagcacatcaatctacacccagagttggatggtcaggaatatgtggtagagtttgacttcctcgggaaggactccatcagatactataacaaggtccctgttgagaaacgagtttttaagaacctacaactatttatggagaacaagcagcccgaggatgatctttttgatagactcaatactggtattctgaataagcatcttcaggatctcatggagggcttgacagccaaggtattccgtacgtacaatgcctccatcacgctacagcagcagctaaaagaactgacagccccggatgagaacatcccagcgaagatcctttcttataaccgtgccaatcgagctgttgcaattctttgtaaccatcagagggcaccaccaaaaacttttgagaagtctatgatgaacttgcaaactaagattgatgccaagaaggaacagctagcagatgcccggagagacctgaaaagtgctaaggctgatgccaaggtcatgaaggatgcaaagacgaagaaggtagtagagtcaaagaagaaggctgttcagagactggaggaacagttgatgaagctggaagttcaagccacagaccgagaggaaaataaacagattgccctgggaacctccaaactcagctatctggaccctaggatcacagtggcttggtgcaagaagtggggtgtcccaattgagaagatttacaacaaaacccagcgggagaagtttgcctgggccattgacatggctgatgaagactatgagttttagccagtctcaagaggcagagttctgtgaagaggaacagtgtggtttgggaaagatggataaactgagcctcacttgccctcgtgcctgggggagagaggcagcaagtcttaacaaaccaacatctttgcgaaaagataaacctggagatattataagggagagctgagccagttgtcctatggacaacttatttaaaaatatttcagatatcaaaattctagctgtatgatttgttttgaattttgtttttattttcaagagggcaagtggatgggaatttgtcagcgttctaccaggcaaattcactgtttcactgaaatgtttggattctcttagctactgtatgcaaagtccgattatattggtgcgtttttacagttagggttttgcaataacttctatattttaatagaaataaattcctaaactcccttccctctctcccatttcaggaatttaaaattaagtagaacaaaaaacccagcgcacctgttagagtcgtcactctctattgtcatggggatcaattttcattaaacttgaagcagtcgtggctttggcagtgttttggttcagacacctgttcacagaaaaagcatgatgggaaaatatttcc

SEQ ID NO: 27 - DNA, 2311 bp, Artificial Sequence
  Topoisomerase I wtih eGFP insertion donor gene
cagcggacactgtgggctgctgctcacttcgtgtggagcacatcaatctacacccagagttggatggtcaggaatatgtggtagagtttgacttcctcgggaaggactccatcagatactataacaaggtccctgttgagaaacgagtttttaagaacctacaactatttatggagaacaagcagcccgaggatgatctttttgatagactcaatactggtattctgaataagcatcttcaggatctcatggagggcttgacagccaaggtattccgtacgtacaatgcctccatcacgctacagcagcagctaaaagaactgacagccccggatgagaacatcccagcgaagatcctttcttataaccgtgccaatcgagctgttgcaattctttgtaaccatcagagggcaccaccaaaaacttttgagaagtctatgatgaacttgcaaactaagattgatgccaagaaggaacagctagcagatgcccggagagacctgaaaagtgctaaggctgatgccaaggtcatgaaggatgcaaagacgaagaaggtagtagagtcaaagaagaaggctgttcagagactggaggaacagttgatgaagctggaagttcaagccacagaccgagaggaaaataaacagattgccctgggaacctccaaactcagctatctggaccctaggatcacagtggcttggtgcaagaagtggggtgtcccaattgagaagatttacaacaaaacccagcgggagaagtttgcctgggccattgacatggctgatgaagactatgagtttaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctatacaagccagtctcaagaggcagagttctgtgaagaggaacagtgtggtttgggaaagatggataaactgagcctcacttgccctcgtgcctgggggagagaggcagcaagtcttaacaaaccaacatctttgcgaaaagataaacctggagatattataagggagagctgagccagttgtcctatggacaacttatttaaaaatatttcagatatcaaaattctagctgtatgatttgttttgaattttgtttttattttcaagagggcaagtggatgggaatttgtcagcgttctaccaggcaaattcactgtttcactgaaatgtttggattctcttagctactgtatgcaaagtccgattatattggtgcgtttttacagttagggttttgcaataacttctatattttaatagaaataaattcctaaactcccttccctctctcccatttcaggaatttaaaattaagtagaacaaaaaacccagcgcacctgttagagtcgtcactctctattgtcatggggatcaattttcattaaacttgaagcagtcgtggctttggcagtgttttggttcagacacctgttcacagaaaaagcatgatgggaaaatatttcctgacttgagtgttcctttttaaatgtgaattttttttttttttaattattttaaaatatttaaacctttttcttgatcttaaagatcgtgtagattggggttggggagggatgaagggcgagtgaatctaaggataatgaaataatcagtgactgaaaccattttcccatcatcctttgttctg

SEQ ID NO: 28 - DNA, 4272 bp, Streptococcus pyogenes
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaa

SEQ ID NO: 29 - DNA, 157 bp, Artificial Sequence
  FtoS sgRNA2.0
gggcgtcctttattgtacttgttttagagctaggccaacatgaggatcacccatgtctgcagggcctagcaagttaaaataaggctagtccgttatcaacttggccaacatgaggatcacccatgtctgcagggccaagtggcaccgagtcggtgct

SEQ ID NO: 30 - DNA, 157 bp, Artificial Sequence
  eGFP sgRNA2.0
agcgggagaagtttgcctgggttttagagctaggccaacatgaggatcacccatgtctgcagggcctagcaagttaaaataaggctagtccgttatcaacttggccaacatgaggatcacccatgtctgcagggccaagtggcaccgagtcggtgct

SEQ ID NO: 31 - DNA, 1579 bp, Artificial Sequence
  TF6 cassette for introduction of knock out mutation in human topoisomerase I gene
ccacgtcatcctataaaagtgaaagaagtgcgctgctgtagtattaccagcgcacttcggcagcggcagcacctcggcagcgtcagtgaacaatgccaagcaagaaaagcatctattttgagtactttcttgctgtcttccagaaaatcaaagaggagaatgaaaaattactgaaagaatatggattctgtattatggataaccacaaagagaggattgctaacttcaagatagagcctcctggactttagcgtggccgcggcaaccaccccaagatgggcatgctgaagagacgaatcatgcccgaggatataatcatcaactgtagcaagcgcagggaggcaaacaatgatatcacaactctcctgacgcgtcatcgtcggctacagcctcgggaattgctacctagctcgagcaagatccaaggagatataacaatggcttcctcctggatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaccacgtcatcctataaaagtgaaagaagtgcgctgctgtagtattaccagcgcacttcggcagcggcagcacctcggcagcgtcagtgaacaatgccaagcaagaaaagc

SEQ ID NO: 32 - DNA, 5106 bp, Artificial Sequence
  dCas9-PCV1 Rep
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaacaggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgctatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacgtgagcgctggaggaggtggaagcggaggaggaggaagcggaggaggaggtagcctcgagaccatgccaagcaagaaaagcggcccgcaaccccataagaggtgggtgttcacccttaataatccttccgaggaggagaaaaacaaaatacgggagcttccaatctccctttttgattattttgtttgcggagaggaaggtttggaagagggtagaactcctcacctccaggggtttgcgaattttgctaagaagcagacttttaacaaggtgaagtggtattttggtgcccgctgccacatcgagaaagcgaaaggaaccgaccagcagaataaagaatattgcagtaaagaaggtcacatacttatcgagtgtggagccccccggaaccaggggaagcgcagcgacctgtctactgctgtgagtacccttttggagacggggtctttggtgactgtagccgagcagttccctgtaacgtatgtgagaaatttccgggggctggctgaacttttgaaagtgagcgggaagatgcagcagcgtgattggaagacagctgtacacgtcatagtgggaccgcccggttgtgggaagagccagtgggcccgtaattttactgagcctagcgacacctactggaagcctagtagaaataagtggtgggatggctatcatggagaagaagttgttgttttggatgatttttatggctggttaccttgggatgatctactgagactgtgtgaccggtatccattgactgtagagactaaaggcggtactgttccttttttggcccgcagtattttgattaccagcaatcaggccccccaggaatggtactcctcaactgctgtcccagccgtagaagctctctatcggaggattactactttgcaattttggaagactgctggagaacaatccacggaggtgcccgaaggccgatttgaagcagtggacccaccctgtgcccttttcccatataaaataaattactag

SEQ ID NO: 33 - DNA, 11029 bp, Artificial Sequence
  TTVori-SERPINA1-TTVori
cggtgaagccacggagggagatctccgcgtcccgagggcgggtgccgaaggtgagtttacacaccgaagtcaaggggcaattcgggctcgggactggccgggctatgggcaaggctctgagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctgtgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctgttcactgctgagcaggagccccttcctgctgaaatggtcctggggagtgcagcaggctctccgggaagaaatctaccatctctcgggcaggagctcaacctgtgtgcaggtacagggagggcttcctcacctggtgcccactcatgcattacgtcagttattcctcatccctgtccaaaggattcttttctccattgtacagctatgaagctagtgctcaaagaagtgaagtcatttaccccaggccccctgccagtaagtgacagggcctggtcacacttgggtttatttattgcccagttcaacaggttgtttgaccataggcgagattctcttccctgcaccctgccgggttgctcttggtcccttattttatgctcccgggtagaaatggtgtgagattaggcagggagtggctcgcttccctgtccctggccccgcaaagagtgctcccacctgccccgatcccagaaatgtcaccatgaagccttcattcttttggtttaaagcttggcctcagtgtccgtacaccatggggtacttggccagatggcgactttctcctctccagtcgccctcccaggcactagcttttaggagtgcagggtgctgcctctgatagaagggccaggagagagcaggttttggagtcctgatgttataaggaacagcttgggaggcataatgaacccaacatgatgcttgagaccaatgtcacagcccaattctgacattcatcatctgagatctgaggacacagctgtctcagttcatgatctgagtgctgggaaagccaagacttgttccagctttgtcactgacttgctgtatagcctcaacaaggccctgaccctctctgggcttcaaactcttcactgtgaaaggaggaaaccagagtaggtgatgtgacaccaggaaagatggatgggtgtgggggaatgtgctcctcccagctgtcaccccctcgccaccctccctgcaccagcctctccacctcctttgagcccagaattcccctgtctaggagggcacctgtctcatgcctagccatgggaattctccatctgttttgctacattgaacccagatgccattctaaccaagaatcctggctgggtgcaggggctctcgcctgtaaccccagcactttgggaggccaaggcaggcggatcaagaggtcaggagttcaagacctgcctggccaacacggtgaaacctcagctctactaaaaatacaaaaattagccaggcgtggtggcacacgcctgtaatcccagctatttgggaagctgagacagaagaatttcttgaacccgggaggtggaggtttcagtgagccgagatcacgccactgcactccaccctggcagataaagcgagactctgtctcaaaaaaaacccaaaaacctatgttagtgtacagagggccccagtgaagtcttctcccagccccactttgcacaactggggagagtgaggccccaggaccagaggattcttgctaaaggccaagtggatagtgatggccctgccagggctagaagccacaacctctggccctgaggccactcagcatatttagtgtccccaccctgcagaggcccaactccctcctgaccactgagccctgtaatgatgggggaatttccataagccatgaaggactgcacaaagttcagttgggaagtgaaagagaaattaaagggagatggaaatatacagcactaattttagcaccgtctttagttctaacaacactagctagctgaagaaaaatacaaacatgtattatgtaatgtgtggtctgttccatttggattacttagaggcacgagggccaggagaaaggtggtggagagaaaccagctttgcacttcatttgttgctttattggaaggaaacttttaaaagtccaagggggttgaagaatctcaatatttgttatttccagctttttttctccagtttttcatttcccaaattcaaggacacctttttctttgtattttgttaagatgatggttttggttttgtgactagtagttaacaatgtggctgccgggcatattctcctcagctaggacctcagttttcccatctgtgaagacggcaggttctacctagggggctgcaggctggtggtccgaagcctgggcatatctggagtagaaggatcactgtggggcagggcaggttctgtgttgctgtggatgacgttgactttgaccattgctcggcagagcctgctctcgctggttcagccacaggccccaccactccctattgtctcagccccgggtatgaaacatgtattcctcactggcctatcacctgaagcctttgaatttgcaacacctgccaacccctccctcaaaagagttgccctctcagatccttttgatgtaaggtttggtgttgagacttatttcactaaattctcatacataaacatcactttatgtatgaggcaaaatgaggaccagggagatgaatgacttgtcctggctcatacacctggaaagtgacagagtcagattagatcccaggtctatctgaagttaaaagaggtgtcttttcacttcccacctcctccatctactttaaagcagcacaaacccctgctttcaaggagagatgagcgtctctaaagcccctgacagcaagagcccagaactgggacaccattagtgacccagacggcaggtaagctgactgcaggagcatcagcctattcttgtgtctgggaccacagagcattgtggggacagccccgtctcttgggaaaaaaaccctaagggctgaggatccttgtgagtgttgggtgggaacagctcccaggaggtttaatcacagcccctccatgctctctagctgttgccattgtgcaagatgcatttcccttctgtgcagcagtttccctggccactaaatagtgggattagatagaagccctccaagggcttccagcttgacatgattcttgattctgatctggcccgattcctggataatcgtgggcaggcccattcctcttcttgtgcctcattttcttcttttgtaaaacaatggctgtaccatttgcatcttagggtcattgcagatgtaagtgttgctgtccagagcctgggtgcaggacctagatgtaggattctggttctgctacttcctcagtgacattgaatagctgacctaatctctctggctttggtttcttcatctgtaaaagaaggatattagcattagcacctcacgggattgttacaagaaagcaatgaattaacacatgtgagcacggagaacagtgcttggcatatggtaagcactacgtacattttgctattcttctgattctttcagtgttactgatgtcggcaagtacttggcacaggctggtttaataatccctaggcacttccacgtggtgtcaatccctgatcactgggagtcatcatgtgccttgactcggggcctggcccccccatctctgtcttgcaggacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggtaaggttgctcaaccagcctgagctgttcccatagaaacaagcaaaaatattctcaaaccatcagttcttgaactctccttggcaatgcattatgggccatagcaatgcttttcagcgtggattcttcagttttctacacacaaacactaaaatgttttccatcattgagtaatttgaggaaataatagattaaactgtcaaaactactgacagctctgcagaacttttcagagcctttaatgtccttgtgtatactgtatatgtagaatatataatgcttagaactatagaacaaattgtaatacactgcataaagggatagtttcatggaacatactttacacgactctagtgtcccagaatcagtatcagttttgcaatctgaaagacctgggttcaaatcctgcctctaacacaattagcttttgacaaaaacaatgcattctacctctttgaggtgctaatttctcatcttagcatggacaaaataccattcttgctgtcaggtttttttaggattaaacaaatgacaaagactgtggggatggtgtgtggcatacagcaggtgatggactcttctgtatctcaggctgccttcctgcccctgaggggttaaaatgccagggtcctgggggccccagggcattctaagccagctcccactgtcccaggaaaacagcataggggaggggaggtgggaggcaaggccaggggctgcttcctccactctgaggctcccttgctcttgaggcaaaggagggcagtggagagcagccaggctgcagtcagcacagctaaagtcctggctctgctgtggccttagtgggggcccaggtccctctccagccccagtctcctccttctgtccaatgagaaagctgggatcaggggtccctgaggcccctgtccactctgcatgcctcgatggtgaagctctgttggtatggcagaggggaggctgctcaggcatctgcatttcccctgccaatctagaggatgaggaaagctctcaggaatagtaagcagaatgtttgccctggatgaataactgagctgccaattaacaaggggcagggagccttagacagaaggtaccaaatatgcctgatgctccaacattttatttgtaatatccaagacaccctcaaataaacatatgattccaataaaaatgcacagccacgatggcatctcttagcctgacatcgccacgatgtagaaattctgcatcttcctctagttttgaattatccccacacaatctttttcggcagcttggatggtcagtttcagcaccttttacagatgatgaagctgagcctcgagggatgtgtgtcgtcaagggggctcagggcttctcagggaggggactcatggtttctttattctgctacactcttccaaaccttcactcacccctggtgatgcccaccttcccctctctccaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtgattccccaacctgagggtgaccaagaagctgcccacacctcttagccatgttgggactgaggcccatcaggactggccagagggctgaggagggtgaaccccacatccctgggtcactgctactctgtataaacttggcttccagaatgaggccaccactgagttcaggcagcgccatccatgctccatgaggaggacagtacccaggggtgaggaggtaaaggtctcgtccctggggacttcccactccagtgtggacactgtcccttcccaatatccagtgcccagggcagggacagcagcaccaccacacgttctggcagaaccaaaaaggaacagatgggcttcctggcaaaggcagcagtggagtgtggagttcaagggtagaatgtccctggggggacgggggaagagcctgtgtggcaaggcccagaaaagcaaggttcggaattggaacagccaggccatgttcgcagaaggcttgcgtttctctgtcactttatcggtgctgttagattgggtgtcctgtagtaagtgatacttaaacatgagccacacattagtgtatgtgtgtgcattcgtgattatgcccatgccctgctgatctagttcgttttgtacactgtaaaaccaagatgaaaatacaaaaggtgtcgggttcataataggaatcgaggctggaatttctctgttccatgccagcacctcctgaggtctctgctccaggggttgagaaagaacaaagaggctgagagggtaacggatcagagagcccagagccaagctgcccgctcacaccagaccctgctcagggtggcattgtctccccatggaaaaccagagaggagcactcagcctggtgtggtcactcttctcttatccactaaacggttgtcactgggcactgccaccagccccgtgtttctctgggtgtagggccctggggatgttacaggctgggggccaggtgacccaacactacagggcaagatgagacaggcttccaggacacctagaatatcagaggaggtggcatttcaagcttttgtgattcattcgatgttaacattctttgactcaatgtagaagagctaaaagtagaacaaaccaaagccgagttcccatcttagtgtgggtggaggacacaggagtaagtggcagaaataatcagaaaagaaaacacttgcactgtggtgggtcccagaagaacaagaggaatgctgtgccatgccttgaatttcttttctgcacgacaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggtgagatcaccctgacgaccttgttgcaccctggtatctgtagggaagaatgtgtgggggctgcagctctgtcctgaggctgaggaaggggccgagggaaacaaatgaagacccaggctgagctcctgaagatgcccgtgattcactgacacgggacgtggtcaaacagcaaagccaggcaggggactgctgtgcagctggcactttcggggcctcccttgaggttgtgtcactgaccctgaatttcaactttgcccaagaccttctagacattgggccttgatttatccatactgacacagaaaggtttgggctaagttgtttcaaaggaatttctgactccttcgatctgtgagatttggtgtctgaattaatgaatgatttcagctaaagatgacacttattttggaaaactaaaggcgaccaatgaacaactgcagttccatgaatggctgcattatcttggggtctgggcactgtgaaggtcactgccagggtccgtgtcctcaaggagcttcaagccgtgtactagaaaggagagagccctggaggcagacgtggagtgacgatgctcttccctgttctgagttgtgggtgcacctgagcagggggagaggcgcttgtcaggaagatggacagaggggagccagccccatcagccaaagccttgaggaggagcaaggcctatgtgacagggagggagaggatgtgcagggccagggccgtccagggggagtgagcgcttcctgggaggtgtccacgtgagccttgctcgaggcctgggatcagccttacaacgtgtctctgcttctctcccctccaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacggtgaagccacggagggagatctccgcgtcccgagggcgggtgccgaaggtgagtttacacaccgaagtcaaggggcaattcgggctcgggactggccgggctatgggcaaggctctga

SEQ ID NO: 34 - DNA, 1331 bp, Human cytomegalovirus
caatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaagtagacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacg

SEQ ID NO: 35 - DNA, 232 bp, Homo sapiens
tttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac